(19) United States
(12) Patent Application Publication
(10) Pub. No.: US 2008/0241130 A1
(43) Pub. Date: Oct. 2, 2008

(54) METHODS AND COMPOSITIONS FOR MODULATING IL-17F/IL-17A BIOLOGICAL ACTIVITY

(76) Inventors: Jill F. Wright, Ashland, MA (US); Spencer C. Liang, Mountain View, CA (US); Frances K. Bennett, Sudbury, MA (US); Mary Collins, Natick, MA (US); Beatriz M. Carreno, Clayton, MO (US)

Correspondence Address: FITZPATRICK CELLA (WYETH), 30 ROCKEFELLER PLAZA, NEW YORK, NY, 10112-3800, US

(21) Appl. No.: 12/057,778

(22) Filed: Mar. 28, 2008

Related U.S. Application Data
(60) Provisional application No. 60/920,591, filed on Mar. 28, 2007.
(60) Provisional application No. 60/922,175, filed on Apr. 5, 2007.

(51) Int. Cl. A61K 39/395; G01N 33/557; A61K 38/17; A61P 11/06

(52) U.S. Cl. 424/130.1; 435/7.92; 514/2

(57) ABSTRACT

The invention provides a novel mouse IL-17F/IL-17A, and further provides uses of such mouse IL-17F/IL-17A in the characterization of the IL-17F/IL-17A heterodimer. The present invention is also related to polynucleotides and polypeptides of the IL-17F/IL-17A signaling pathway, and targeting of the IL-17F/IL-17A signaling pathway in methods of treating IL-17F/IL-17A-associated disorders. The invention thus provides methods of using isolated IL-17F/IL-17A heterodimer, e.g., in a mouse model of airway inflammation, and specific or selective IL-17F/IL-17A modulators (e.g., signaling agonists or signaling antagonists (e.g., specific or selective antagonistic antibodies, specific or selective antagonistic small molecules, etc.)). The invention also provides methods of screening for compounds capable of modulating IL-17F/IL-17A biological activity, e.g., IL-17F/IL-17A signaling antagonists (e.g., using the mouse model of airway inflammation), as well as methods of identifying whether the IL-17F/IL-17A modulator is a specific IL-17F/IL-17A modulator. The invention is also directed to novel methods for diagnosing, prognosing, monitoring, preventing, and/or treating IL-17F/IL-17A-associated disorders, including, but not limited to, inflammatory disorders (e.g., arthritis (including rheumatoid arthritis), psoriasis, systemic lupus erythematosus, and multiple sclerosis), respiratory diseases (e.g., airway inflammation, chronic obstructive pulmonary disease, cystic fibrosis, asthma, allergy), transplant rejection (including solid organ transplant rejection), and inflammatory bowel diseases or disorders (e.g., ulcerative colitis, Crohn's disease). The present invention is further directed to novel therapeutics and therapeutic targets identified by methods of screening of the invention, and uses of such identified therapeutics in methods of treatment and prevention of IL-17F/IL-17A-associated disorders.

This application claims the benefit of U.S. Provisional Application Ser. No. 60/920,591, filed Mar. 28, 2007, and U.S. Provisional Application Ser. No. 60/922,175, filed Apr. 5, 2007, both of which are incorporated herein by reference in their entireties.

### BACKGROUND OF THE INVENTION

1. Field of the Invention

This invention relates to the discoveries that triggering the IL-17F/IL-17A signaling pathway induces inflammation, e.g., airway inflammation, and that blocking the IL-17F/IL-17A signaling pathway prevents and/or treats IL-17F/IL-17A-associated disorders, e.g. inflammation, e.g., airway inflammation. Thus, the invention relates to IL-17F/IL-17A signaling antagonists, e.g., antagonistic antibodies to IL-17F/IL-17A and fragments thereof, soluble receptors, small molecules, inhibitory polynucleotides, etc. The antibodies and other IL-17F/IL-17A signaling antagonists are useful in methods of diagnosing, prognosing, monitoring, preventing, and/or treating IL-17F/IL-17A-associated disorders, e.g., inflammatory disorders (e.g., autoimmune diseases (e.g., arthritis), respiratory diseases (e.g., airway inflammation, COPD, cystic fibrosis, asthma, allergy, pulmonary exacerbation (e.g., due to bacterial infection)), inflammatory bowel disorders (e.g., ulcerative colitis, Crohn's disease)), and transplant rejection.

2. Related Background Art

The IL-17 cytokine family consists of six structurally related proteins (IL-17A, IL-17B, IL-17C, IL-17D, IL-17E, and IL-17F), the functions of which are now being elucidated. The best-characterized molecule of this family is IL-17A. IL-17A is expressed primarily by Th17 cells, a subset of CD4⁺ T cells, and is known to signal through two receptors, IL-17RA (also known in the art as IL-17R) and IL-17RC (Aggarwal et al. (2003) J. Biol. Chem. 278:1910-14; Langrish et al. (2005) J. Exp. Med. 201:233-40; Veldhoen et al. (2006) Immunity 24:179-89; Bettelli et al. (2006) Nature 441:235-38; Mangan et al. (2006) Nature 441:231-34; Yao et al. (1995) Immunity 3:811-21; Toy et al. (2006) J. Immunol. 177:36-39). Although these receptors are expressed broadly, IL-17A is believed to act primarily on parenchymal cells such as fibroblasts, epithelial cells, and endothelial cells. Signaling by IL-17A increases matrix metalloproteinase and proinflammatory cytokine expression (as reviewed in Kolls and Linden (2004) Immunity 21:467-76; Weaver et al. (2007) Annu. Rev. Immunol. 25:821-52). IL-17A also acts to recruit neutrophils to peripheral sites through the induction of CXC chemokines and G-CSF. The expression of IL-17A is enhanced in several pulmonary diseases in which neutrophils are present, including severe asthma, chronic obstructive pulmonary disease (COPD), and cystic fibrosis (Barczyk et al. (2003) Respir. Med. 97:726-33; Molet et al. (2001) J. Allergy Clin. Immunol. 108:430-38; Wong et al. (2001) Clin. Exp. Immunol. 125:177-83; Shen et al. (2004) Zhonghua Nei Ke Za Zhi 43:888-90; McAllister et al. (2005) J. Immunol. 175:404-12). As a result, considerable attention has been given to the role of IL-17A in the pathogenesis of airway disease.

Administration of IL-17A into the airways is sufficient to induce a significant increase in neutrophils through enhanced CXCL1 (KC) and CXCL2 (MIP-2) expression (Laan et al. (1999) J. Immunol. 162:2347-52; Ferretti et al. (2003) J. Immunol. 170:2106-12). In a model of LPS-driven airway inflammation, neutralization of IL-17A significantly reduces neutrophil number (Ferretti et al. (2003) supra; Miyamoto et al. (2003) J. Immunol. 170:4665-72). These data point to an important role for IL-17A in regulating airway inflammation and neutrophil recruitment.

Of the remaining five IL-17 family members, IL-17F is most closely related to IL-17A. The two molecules share a high degree of homology (about 57% similarity and 52% identity), and are syntenic (both are located on mouse chromosome 1A4). Like IL-17A, IL-17F mRNA and protein have been detected in Th17 cells (Langrish et al. (2005) supra; Liang et al. (2006) J. Exp. Med. 203:2271-79). IL-17F exists as a homodimer, adopting a cysteine-knot motif formed through the interactions of four cysteines, one of which is responsible for the interchain bonding (Hymowitz et al. (2001) EMBO J. 20:5332-41). These cysteines are also highly conserved in IL-17A, suggesting that IL-17A has a homodimeric structure similar to IL-17F. IL-17A and IL-17F are also believed to share the same receptors, suggesting similar functions (Toy et al. (2006) supra; Kramer et al. (2006) J. Immunol. 176:711-15). The majority of IL-17F functional studies have examined the effects of the human cytokine. In vitro studies using recombinant human IL-17F have demonstrated that IL-17F can induce G-CSF and CXCL1 from primary human epithelial cells (McAllister et al. (2005) supra). Overexpression of human IL-17F using adenoviral vectors, or of mouse IL-17F using pulmonary gene transfer in mouse airways, induces a significant increase in neutrophil numbers and chemokine expression (Hurst et al. (2002) J. Immunol. 169:443-53). Although these studies point to overlapping functions in the airways for IL-17A and IL-17F, there are also likely to be nonredundant features. Consistent with this, IL-17A-deficient mice have a profound phenotype that does not appear to be compensated by IL-17F expression (Nakae et al. (2003) J. Immunol. 171:6173-77).

The high sequence homology between IL-17A and IL-17F and the conserved location of their cysteines suggested that a heterodimer of IL-17A and IL-17F could exist; the coexpression of IL-17A and IL-17F by Th17 cells further supported this possibility. Recently, the existence of human IL-17F/IL-17A heterodimer has been demonstrated using biochemical and physiochemical methods (Wright et al. (2007) J. Biol. Chem. 282:13447-55; see also U.S. patent application Ser. No. 11/353,161, hereby incorporated by reference herein in its entirety). Mass spectrometry analysis of natural IL-17F/IL-17A heterodimer produced by primary human CD4⁺ T cells has shown the existence of interchain disulfide-linked peptides, containing one peptide from IL-17F and one peptide from IL-17A. This suggests the existence of IL-17F/IL-17A heterodimer that may have novel functions.

In addition to producing IL-17A and IL-17F, Th17 cells also produce IL-22, an IL-10 family member (Liang et al. (2006) supra; Chung et al. (2006) Cell Res. 16:902-07; Zheng et al. (2007) Nature 445:648-51; Renauld (2003) Nat. Rev. Immunol. 3:667-76). IL-22 acts on epithelial cells and some fibroblast cells, and has been shown to play a role in inflammation. IL-22 induces gene expression indicative of an acute phase response (Wolk et al. (2004) Immunity 21:241-54). Similar to IL-17A and IL-17F, IL-22 can also enhance the expression of matrix metalloproteinases, chemokines, and cytokines in certain tissues (Wolk et al. (2004) supra; Ikeuchi et al. (2005) Arthritis Rheum. 52:1037-46; Andoh et al. (2005) Gastroenterology 129:969-84; Boniface et al. (2005) J. Immunol. 174:3695-02). The coexpression of IL-22 with IL-17A and IL-17F by Th17 cells suggests that these cytokines may function together to mediate inflammation. However, prior to the invention disclosed herein, neither the receptor(s) for human IL-17F/IL-17A heterodimer nor mouse IL-17F/IL-17A heterodimer was known and available to study the biological activity of IL-17F/IL-17A.

### SUMMARY OF THE INVENTION

The invention provides the receptor(s) for the human IL-17F/IL-17A heterodimer, and thus, the biological activities of human IL-17F/IL-17A. The invention also provides a novel mouse protein that is an IL-17F/IL-17A heterodimer. Also disclosed herein is the characterization of the expression of mouse IL-17A, mouse IL-17F/IL-17A, and mouse IL-17F by mouse Th17 cells, comparison of the functions and activities of mouse IL-17A, mouse IL-17F/IL-17A, and mouse IL-17F in vitro, and comparison of the roles played by mouse IL-17A, mouse IL-17F/IL-17A, and mouse IL-17F in neutrophil recruitment and chemokine production in vivo. Additionally, a Th17 cell adoptive transfer model to examine the essential roles of these cytokines in regulating airway inflammation is established. It is demonstrated herein that mIL-17F and mIL-22 do not have overlapping functions with mIL-7A or mIL-17F/IL-17A in the airways and that mouse IL-17F/IL-17A is biologically active and can induce neutrophil recruitment in vivo. Thus, the present invention provides the IL-17F/IL-17A signaling pathway as a new target for the prevention and/or treatment of various diseases, e.g., airway inflammation, arthritis, asthma, allergy, COPD, cystic fibrosis, Crohn's disease, etc.

The present invention provides various methods and compositions related to IL-17F/IL-17A heterodimer and IL-17F/IL-17A signaling. Thus in at least one embodiment, the invention provides a method of screening for compounds capable of antagonizing IL-17F/IL-17A signaling comprising the steps of contacting a sample containing IL-17F/IL-17A and IL-17R with one of a plurality of test compounds; and determining whether the biological activity of IL-17F/IL-17A in the sample is decreased relative to the biological activity of IL-17F/IL-17A in a sample not contacted with the test compound, whereby such a decrease in the biological activity of IL-17F/IL-17A in the sample contacted with the test compound identifies the compound as an IL-17F/IL-17A signaling antagonist. In at least one other embodiment, the method further comprises a first or a last step of identifying whether the IL-17F/IL-17A signaling antagonist is a specific IL-17F/IL-17A signaling antagonist. In at least one other embodiment, the step of identifying further comprises the steps of contacting a sample containing IL-17A and IL-17R with the IL-17F/IL-17A signaling antagonist; determining whether the biological activity of IL-17A in the sample is decreased relative to the biological activity of IL-17A in a sample not contacted with the IL-17F/IL-17A signaling antagonist; contacting a sample containing IL-17F and IL-17R with the IL-17F/IL-17A signaling antagonist; and determining whether the biological activity of IL-17F in the sample is decreased relative to the biological activity of IL-17F in a sample not contacted with the IL-17F/IL-17A signaling antagonist, whereby a failure of the IL-17F/IL-17A signaling antagonist to decrease the biological activity of both IL-17F and IL-17A identifies the IL-17F/IL-17A signaling antagonist as a specific IL-17F/IL-17A signaling antagonist. In at least one other embodiment, the invention provides a compound identified by one of these methods.

In at least one embodiment, the invention provides a method of screening for compounds capable of antagonizing IL-17F/IL-17A signaling comprising the steps of contacting a sample containing IL-17F/IL-17A and IL-17RC with one of a plurality of test compounds; and determining whether the biological activity of IL-17F/IL-17A in the sample is decreased relative to the biological activity of IL-17F/IL-17A in a sample not contacted with the test compound, whereby such a decrease in the biological activity of IL-17F/IL-17A in the sample contacted with the test compound identifies the compound as an IL-17F/IL-17A signaling antagonist. In at least one other embodiment, the method further comprises a first or a last step of identifying whether the IL-17F/IL-17A signaling antagonist is a specific IL-17F/IL-17A signaling antagonist. In at least one other embodiment, the step of identifying further comprises the steps of contacting a sample containing IL-17A and IL-17RC with the IL-17F/IL-17A signaling antagonist; determining whether the biological activity of IL-17A in the sample is decreased relative to the biological activity of IL-17A in a sample not contacted with the IL-17F/IL-17A signaling antagonist; contacting a sample containing IL-17F and IL-17RC with the IL-17F/IL-17A signaling antagonist; and determining whether the biological activity of IL-17F in the sample is decreased relative to the biological activity of IL-17F in a sample not contacted with the IL-17F/IL-17A signaling antagonist, whereby the failure of the IL-17F/IL-17A signaling antagonist to decrease the biological activity of both IL-17F and IL-17A identifies the IL-17F/IL-17A signaling antagonist as a specific IL-17F/IL-17A signaling antagonist. In at least one other embodiment, the invention provides a compound identified by one of these methods.

In at least one embodiment, the invention provides a method of inhibiting IL-17F/IL-17A biological activity in a subject, the method comprising administering to the subject an IL-17F/IL-17A signaling antagonist. In at least one other embodiment, the invention provides a method of inhibiting GRO-A secretion in a cell population comprising administering to the cell population an IL-17F/IL-17A signaling antagonist. In at least one other embodiment, the invention provides a method of treating a subject at risk for, or diagnosed with, an IL-17F/IL-17A-associated disorder comprising administering to the subject a therapeutically effective amount of an IL-17F/IL-17A signaling antagonist. In at least one further embodiment, the IL-17F/IL-17A signaling antagonist is a specific IL-17F/IL-17A signaling antagonist. In at least one other further embodiment, the IL-17F/IL-17A signaling antagonist is selected from the group consisting of an antagonistic small molecule and an antagonistic antibody. In at least one other embodiment, the antagonistic small molecule is specific for IL-17F/IL-17A. In at least one other embodiment, the antagonistic antibody is specific for IL-17F/IL-17A. In at least one other embodiment, the IL-17F/IL-17A signaling antagonist is a compound identified by one of the methods of the present invention. In at least one other embodiment, the IL-17F/IL-17A-associated disorder is an inflammatory disorder. In at least one other embodiment, the IL-17F/IL-17A-associated disorder is a respiratory disorder. In at least one further embodiment, the respiratory disorder is selected from the group consisting of airway inflammation, asthma, and COPD.

In at least one embodiment, the invention provides a pharmaceutical composition comprising an IL-17F/IL-17A signaling antagonist and a pharmaceutically acceptable carrier. In at least one other embodiment, the IL-17F/IL-17A signaling antagonist is selected from the group consisting of an antagonistic small molecule and an antagonistic antibody. In at least one other embodiment, the antagonistic small molecule is specific for IL-17F/IL-17A. In at least one other embodiment, the antagonistic antibody is specific for IL-17F/IL-17A. In at least one other embodiment, the IL-17F/IL-17A signaling antagonist is a compound identified by one of the methods of the present invention.

In at least one embodiment, the invention provides an isolated antibody capable of specifically binding IL-17F/IL-17A heterodimer. In at least one other embodiment, the antibody inhibits IL-17F/IL-17A signaling. In at least one other embodiment, the invention provides a small molecule capable of specifically binding IL-17F/IL-17A heterodimer. In at least one other embodiment, the small molecule inhibits IL-17F/IL-17A signaling.

In at least one embodiment, the invention provides a method of inducing airway inflammation in a subject comprising administering to the subject IL-17F/IL-17A. In at least one other embodiment, the subject is a mouse.

### BRIEF DESCRIPTION OF THE DRAWINGS

FIG. 1 demonstrates the binding (O.D. 450 nm; y-axes) of increasing concentrations (ng/ml Cytokine; x-axes) of human IL-17A (hIL-17A; ♦), human IL-17F (hIL-17F; ), or human IL-17F/IL-17A (hIL-17F/A; Δ) cytokines to the IL-17R.Fc receptor (FIG. 1A) or IL-17RC.Fc receptor (FIG. 1B), as measured by ELISA.

FIG. 2 demonstrates human IL-17A, human IL-17F, and human IL-17F/IL-17A functional (biological) activity represented by GRO-A release from BJ cells (pg/ml GRO-alpha; y-axis) after treatment with increasing concentrations (ng/ml of Cytokine; x-axis) of human IL-17A (IL-17A; ♦), human IL-17F (IL-17F; ▴), or human IL-17F/IL-17A (IL-17F/A; ◯). GRO-α release was measured by ELISA.

FIG. 3A and FIG. 3B demonstrate relative GRO-A release (Relative Response; y-axes) from BJ cells induced by 1 ng/ml human IL-17A, 50 ng/ml human IL-17F, or 5 ng/ml human IL-17F/IL-17A cytokine in the presence of (FIG. 3A) soluble receptor fusion proteins hIL-17R.Fc, hIL-17RC.Fc, or the combination of hIL-17R.Fc and hIL-17RC.Fc, and (FIG. 3B) anti-hIL-17R and anti-hIL-17RC antibodies. Control antibodies were included in both experiments.

FIG. 4A and FIG. 4B demonstrate the effect of four IL-17R siRNAs (R-1, R-2, R-3, and R-4; x-axis) and four IL-17RC siRNAs (RC-1, RC-2, RC-3, RC-4; x-axis), respectively, on hIL-17A- and hIL-17F-induced GRO-α release (Relative Response; y-axes) from BJ cells. “Taqman” represents relative amount of either IL-17R (FIG. 4A) or IL-17RC (FIG. 4B) mRNA under the treatment conditions. “Mock” represents treatment with culture medium and transfection reagent only. “NTC1” represents transfection with nonspecific control siRNA. The effect of siRNA transfection on hIL-17R and hIL-17RC expression in HEK293 cells transfected with hIL-17R and hIL-17RC, respectively, is demonstrated by Western blot in FIG. 4C. Actin Western blot represents a protein-loading control.

FIG. 5 represents the effects of IL-17R siRNA (R-3 and R-4) and IL-17RC siRNA (RC-2 and RC-4) treatment on GRO-α release (pg/ml GROa; y-axes) in BJ cells treated with decreasing concentrations (x-axes) of human IL-17A (FIG. 5A), human IL-17F (FIG. 5B), or human IL-17F/IL-17A (FIG. 5C). NTC1 represent transfection with nonspecific control siRNA.

Shown in FIG. 6A are flow cytometric dot plots of CD4⁺ CD62L⁺ (naïve) DO11 T cells stained intracellularly for IL-17F (y-axes) and IL-17A (x-axes) after a four-day activation with irradiated splenocytes, 1 μg/ml OVA₃₂₃₋₃₃₉, and one of the following three cytokine treatments: TGF-β, IL-6, or both TGF-β and IL-6 (TGF-β, IL-6). Shown in FIG. 6B are flow cytometric dot plots of CD4⁺ CD62L⁺ (naïve) DO11 T cells activated with irradiated splenocytes, 1 μg/ml OVA₃₂₃₋₃₃₉, and both TGF-β and IL-6 that were stained for intracellular mouse IL-17F (y-axes) and mouse IL-17A (x-axes) after Day 1, Day 2, Day 3, or Day 4 of activation. All plots are gated on CD4⁺ DO11 T cells. Data are representative of three separate experiments.

Shown in FIG. 7A are Western blots of purified recombinant mouse IL-17F/IL-17A, mouse IL-17A, or mouse IL-17F proteins (35 ng per lane) analyzed with (left panel (i)) anti-IL-17A antibody or (right panel (ii)) anti-IL-17F antibody. The size of mouse IL-17F/IL-17A is modified due to the presence of tags used in its purification (see Example 2.2.2). FIGS. 7B, 7C, and 7D demonstrate detection (O.D.; y-axes) of various concentrations (ng/ml; x-axes) of purified recombinant mouse IL-17A (open squares), mouse IL-17F/IL-17A (filled circles) or mouse IL-17F (filled triangles) by mouse IL-17A (FIG. 7B), mouse IL-17F/IL-17A (FIG. 7C), or mouse IL-17F (FIG. 7D) quantitation ELISA. Insets represent an expanded view of the lower concentrations, with the dashed line representing the limit of detection. FIG. 7E demonstrates mouse IL-17A (open columns), mouse IL-17F/IL-17A (hatched columns) or mouse IL-17F (filled columns) production (ng/ml; y-axis) by CD4⁺ CD62L⁺ DO11 T cells that were activated in a primary activation with irradiated splenocytes, 1 μg/ml OVA₃₂₃₋₃₃₉, and the indicated cytokines (x-axis) for seven days. FIG. 7F demonstrates mouse IL-17A (open columns), mouse IL-17F/IL-17A (hatched columns) or mouse IL-17F (filled columns) production (ng/ml; y-axis) by CD4⁺ CD62L⁺ DO11 T cells that were activated in a primary activation with irradiated splenocytes, 1 μg/ml OVA₃₂₃₋₃₃₉, and the indicated cytokines (x-axis; “Primary” (i.e., TGF-β, IL-6, and IL-10; or TGF-β, IL-6, IL-1β, and IL-23)) for seven days, harvested, rested overnight, and restimulated for a secondary activation (x-axis; “Secondary”) with either irradiated splenocytes, IL-2 and 1 μg/ml OVA₃₂₃₋₃₃₉ alone (−); or addition of the following: IL-23, anti-IFN-γ antibody (αIFN-γ), and anti-IL-4 antibody (αIL-4). For FIG. 7E and FIG. 7F, conditioned medium was analyzed for IL-17A, IL-17F/IL-17A, and IL-17F on day 4 after each activation, data shown are average ±SD, and * denotes <1 ng/ml of IL-17A. FIGS. 7E and 7F are representative of at least three experiments.

FIG. 8 demonstrates CXCL1 concentration (CXCL1 (pg/ml); y-axes) of conditioned media isolated from murine lung epithelial (MLE-12) cells incubated for 24 hours with (FIG. 8A) mouse IL-17A (open squares), mouse IL-17F/IL-17A (filled circles), and mouse IL-17F (filled triangles) at various concentrations (Cytokine (ng/ml); x-axis); (FIG. 8B) various concentrations of mouse IL-17F (IL-17F (ng/ml); x-axis) preincubated with 50 μg/ml of two different anti-IL-17F antibodies (αIL-17F(RK015-01) (filled circles) or αIL-17F(RK016-17) (filled triangles)) or rat IgG1 (open squares); or (FIG. 8C) 200 ng/ml mouse IL-17F/IL-17A preincubated with 80 μg/ml of the indicated antibody or antibodies (x-axis). For FIGS. 8A, 8B, and 8C, the dashed line represents the basal amount of CXCL1 produced by MLE-12 cells in the absence of exogenous cytokines. All data are represented as average ±SD, and are representative of three experiments.

FIG. 9 represents: (FIG. 9A) concentrations of mouse IL-17F/IL-17A (IL-17F/IL-17A (pg/ml); y-axis) and mouse IL-22 (IL-22 (pg/ml);y-axis) in BAL fluid; (FIG. 9B) differential cell counts (Cells (×10⁵); y-axis) for neutrophils, eosinophils, lymphocytes, and monocytes (x-axis) in BAL fluid; or (FIG. 9C) H&E histology at 40× magnification of lungs (“A” indicates airway lumen and “V” indicates blood vessel) isolated from control naïve BALB/c animals that received 2.5×10⁶ Th17 cells, and were subsequently challenged 24 hours later with PBS intranasally once a day for three consecutive days (open columns (in FIGS. 9A and 9B); Th17/PBS), control naïve BALB/c animals that did not receive Th17 cells, and were subsequently challenged with 75 μg of ovalbumin (OVA) intranasally once a day for three consecutive days (hatched columns; no cells/OVA), or naïve BALB/c animals that received 2.5×10⁶ Th17 cells, and were subsequently challenged 24 hours later with 75 μg of OVA intranasally once a day for three consecutive days (filed columns; Th17/OVA). For FIGS. 9A and 9B, data are average ±SEM. For FIGS. 9A, 9B, and 9C, n=5-6 mice per group, and data are representative of at least two experiments.

FIG. 10 demonstrates: the (FIG. 10A) number of neutrophils (Cells (×10⁵); y-axis), (FIG. 10B) the concentration of mouse CXCL1 (ng/ml; y-axis), or (FIG. 10C) the concentration of CXCL5 (ng/ml; y-axis) in BAL fluid isolated from control animals that did not receive Th17 cells (−; x-axes) but were subsequently challenged intranasally with ovalbumin (OVA; +) or from animals that received Th17 cells (+), were untreated (−) or treated (+) with neutralizing antibody (mAb) to mouse IL-17A (Anti IL-17A (50104)), neutralizing antibody to mouse IL-17F (Anti IL-17F (RK015-01)), neutralizing antibody to mouse IL-22 (Anti IL-22 (Ab-01)) or appropriate isotype control antibodies (IgG2a or IgG1), and subsequently challenged intranasally with ovalbumin (OVA; +). The BAL fluid was collected 24 hours after the last ovalbumin challenge. Data are average ±SEM, n=8-9 mice per group, and are representative of two to three experiments, depending on the antibody.

FIGS. 11A-11E show the number of Neutrophils (cells; y-axes) (A-C), CXCL1 concentration (pg/ml; y-axes) (A, B and D), and CXCL5 concentration (pg/ml; y-axes) (A, B and E) in BAL fluid isolated 24 hours after mice were administered (A) one intranasal dose of 1.5 μg of mouse IL-17A or mouse IL-17F (x-axes), (B) intranasal doses of 1.5 μg of mouse IL-17A or mouse IL-17F (x-axes) daily for three consecutive days, or (C-E) one intranasal dose of 1.5 μg of mouse IL-17A, mouse IL-17F/IL-17A, mouse IL-17F, or mouse IL-22 (x-axes). Control animals were administered phosphate buffered saline (PBS). Data are average ±SEM, n=7, and are representative of two experiments. All p values are calculated relative to control animals receiving only PBS.

FIGS. 12A and 12B demonstrate the results of ELISAs measuring the optical density (O.D.; y-axes) of different concentrations (Cytokine (ng/ml); x-axes) of recombinant mouse IL-17A (open squares) or mouse IL-17F/IL-17A (filled circles) using one of two different anti-IL-17F antibodies as the capture antibody: (A) Anti-IL-17F (RK015-01) or (B) Anti-IL-17F (RK016-17) bound onto ELISA plates that had been precoated with goat anti-rat IgG1 and using goat anti-mouse IL-17A as the detection reagent. FIG. 12C demonstrates the concentration of CXCL1 (“CXCL-1 pg/ml”; y-axis) in medium isolated from MLE-12 cells cultured for 24 hours with 200 ng/ml of IL-17A that had been preincubated with 50 μg/ml of one of the following antibodies (x-axis): IgG2a, anti-mouse IL-17A (anti-mIL17A(50104)), rat IgG1 (rIgG1), anti-mouse IL-17F (anti-mIL17F(RK015-01)), and anti-mouse IL-17F (anti-mIL17F(RK016-17)).

FIG. 13 shows the number of neutrophils (Cells (×10⁵); left panel, y-axis) and the concentration of CXCL5 (ng/ml; right panel, y-axis) in BAL fluid isolated from control animals that did not receive Th17 cells (−; x-axes) but were subsequently challenged intranasally with ovalbumin (OVA i.n.; +) or from animals that received Th17 cells (+), were not treated (−) or treated (+) with neutralizing monoclonal antibody (mAb) to mouse IL-17F (Anti-IL-17F (RK016-17)) or an appropriate isotype control antibody (IgG1), and subsequently challenged intranasally with ovalbumin. The BAL fluid was collected 24 hours after the last ovalbumin challenge. Data are average ±SEM, n=8-9 mice per group, and are representative of two to three experiments, depending on the antibody.

### DETAILED DESCRIPTION OF THE INVENTION

The present invention is based, in part, on two studies; one study elucidated the signaling pathway for human (h) IL-17F/IL-17A, and the other uncovered a novel mouse (m) IL-17F/IL-17A heterodimer and its biological activities in vivo. These studies taken alone or together provide a basis for targeting the IL-17F/IL-17A signaling pathway in methods of treating IL-17F/IL-17A-associated disorders.

The inventors demonstrated that hIL-17F/IL-17A, the recently identified member of the IL-17 cytokine family, utilized the same receptor complex as hIL-17F and hIL-17A. The inventors investigated the roles of human IL-17R (hIL-17R) and human IL-17RC (hIL-17RC) receptors on the biological activity of human IL-17F (hIL-17F), human IL-17A (hIL-17A), and human IL-17F/IL-17A (hIL-17F/IL-17A) heterodimer. Using various approaches, including Biacore and siRNA, the inventors characterized the interactions and kinetic parameters for human IL-17F, hIL-17A, and hIL-17F/IL-17A binding to hIL-17R and hIL-17RC. Using soluble hIL-17R and hIL-17RC receptors, antibodies to these receptors, and receptor siRNA molecules directed to these receptors, the inventors demonstrated that hIL-17R and, to a lesser extent, hIL-17RC are required for the biological activity of the three hIL-17 cytokines (i.e., hIL-17A, hIL-17F, and hIL-17F/IL-17A). Furthermore, the inventors provide evidence that hIL-17R dominates in hIL-17A- and hIL-17F/IL-17A-mediated responses, whereas hIL-17RC appears to be more important than IL-17R for the biological activity of hIL-17F. Thus, the present invention is based, in part, on the following findings: (1) hIL-17A, hIL-17F, and hIL-17F/IL-17A bind to the hIL-17RC receptor with the same affinity; (2) hIL-17A has the highest affinity for the hIL-17R receptor, followed by the hIL-17F/IL-17A heterodimer, followed by hIL-17F; (3) hIL-17A, hIL-17F, and hIL-17F/IL-17A induce release of proinflammatory cytokines (e.g., GRO-α); (4) hIL-7R and hIL-17RC are required for hIL-17F, hIL-17A, and hIL-17F/IL-17A signaling. The finding that hIL-17F/IL-17A binds hIL-17R and hIL-17RC provides this signaling pathway as a target in methods of treating, e.g., inflammatory diseases, respiratory disorders, autoimmune diseases, and transplant rejection.

It was previously reported that hIL-17R is required for the functional activity of hIL-17A and hIL-17F (McAllister et al. (2005) J. Immunol. 175:404-12). Also, it has been recently been shown that hIL-17R self-associates on the cell surface in the absence of ligand, and that receptor association is reduced in the presence of hIL-17A due to a conformational change (Kramer et al. (2006) J. Immunol. 176:711-15). Another study proposed that ligand binding alters the conformation of hIL-17R to facilitate a functional, heterotypic interaction with hIL-17RC (Toy et al. (2006) J. Immunol. 177:36-39). Studies related to the instant invention suggest a role for an IL-17R/IL-17RC cell-surface receptor complex in the biological activity of the cytokines.

Further the inventors discovered that mouse Th17 cells also produce a mouse IL-17F/IL-17A (mIL-17F/IL-17A) heterodimeric protein. Whereas naïve CD4⁺ T cells differentiating towards Th17 expressed mIL-17F/IL-17A in higher amounts than mIL-17A (mIL-17A) homodimer and in lower amounts than mouse IL-17F (mIL-17F) homodimer, differentiated Th17 cells expressed mIL-17F/IL-17A in comparable amounts to both mouse homodimers. These results indicate that the relative amounts of IL-17A, IL-17F/IL-17A, and IL-17F produced by Th17 cells are regulated depending on the stage of differentiation. These in vitro observations suggest a restriction of IL-17A expression in vivo by differentiating Th17 cells during the early phase of the adaptive immune response. Recently, RORγt and STAT3 transcription factors have been identified to be regulators of Th17 differentiation (Ivanov et al. (2006) Cell 126:1121-33; Chen et al. (2006) Proc. Natl. Acad. Sci. U.S.A. 103:8137-42). The distinct profiles observed may be related to the differential expression of these, or other unidentified, transcription factors in naïve cells versus differentiated Th17 cells. There may also be differences in the transcriptional accessibility between the loci encoding IL-17A and IL-17F.

The inventors also demonstrated that in vitro, mIL-17F/IL-17A was more potent than mIL-17F and less potent than mIL-17A. Neutralization of mIL-17F/IL-17A with a mIL-17A-specific antibody, and not with a mIL-17F-specific antibody, reduced the majority of mIL-17F/IL-17A activity. This suggests that mIL-7A and mIL-17F/IL-17A have at least one conserved receptor-binding site that is blocked by the mIL-17A-specific antibody.

To study these cytokines in vivo, the inventors established a Th17 cell adoptive transfer model characterized by increased neutrophils in the airways. A mIL-17A-specific antibody completely prevented Th17 cell induced neutrophilia and CXCL5 expression whereas antibodies specific for mIL-17F or mIL-22, the latter a cytokine also produced by Th17 cells, had no effects. Direct administration of mIL-17A or mIL-17F/IL-17A protein into the airways, and not mIL-17F or mIL-22, significantly increased neutrophils and chemokine expression. Taken together, the mouse data demonstrate that mIL-17F and mIL-17A do not have identical functions. Moreover, the mouse data demonstrate the expression and function of a novel mIL-17F/IL-17A heterodimer and show an in vivo role for this cytokine in airway inflammation, e.g., airway neutrophilia. The IL-17F/IL-17A heterodimer represents a new protein capable of mediating certain functions of Th17 cells, and adds another dimension of possible functional cooperation among cytokines produced in the Th17 lineage.

### Polynucleotides and Polypeptides of IL-17A, IL-17F, IL-17R, and IL-17RC

Unless otherwise indicated, and unless context requires otherwise, the terms “IL-17A,” “IL-17F,” “IL-17F/IL-17A,” “IL-17R,” (or “IL-17RA”) and “IL-17RC,” without any species designation of human (h) or mouse (m), broadly refers to the respective IL-17A, IL-17F, and IL-17F/IL-17A cytokines and respective IL-17R (or IL-17RA) and IL-17RC receptors of both human and mouse species, as well as other mammalian species.

The present invention provides further characterization of the human IL-17F/IL-17A signaling pathway, i.e., determination of human IL-17R and human IL-17RC as common receptors for human IL-17A, human IL-17F, and human IL-17F/IL-17A. As such, the present invention relates to human IL-17F, human IL-17A, human IL-17R, and human IL-17RC polynucleotides and polypeptides. The present invention also provides a novel mouse IL-17F/IL-17A heterodimer. As such, the present invention relates to mouse IL-17F and mouse IL-17A polynucleotides and polypeptides.

IL-17A nucleotide and amino acid sequences are known in the art and are provided. The nucleotide sequence of a cDNA encoding human IL-17A is set forth as SEQ ID NO:1, which includes a poly(A) tail. Nucleic acid residues 54-521 represent the open reading frame of SEQ ID NO:1, which includes a stop codon. The amino acid sequence of full-length human IL-17A protein encoded by SEQ ID NO:1 is set forth as SEQ ID NO:2. The nucleotide sequence of a cDNA encoding mouse IL-17A is set forth as SEQ ID NO:34. The amino acid sequence of full-length mouse IL-17A protein encoded by SEQ ID NO:34 is set forth as SEQ ID NO:35.

IL-17F nucleotide and amino acid sequences are known in the art and are provided. The nucleotide sequence of cDNA encoding human IL-17F is set forth as SEQ ID NO:3. The amino acid sequence of full-length human IL-17F protein coded by that nucleotide sequence is set forth as SEQ ID NO:4. The amino acid sequence of mature IL-17F protein corresponds to a protein beginning at about amino acid 31 of SEQ ID NO:4 (see, e.g., U.S. patent application Ser. No. 10/102,080, incorporated herein in its entirety by reference). The nucleotide sequence of a cDNA encoding mouse IL-17F is set forth as SEQ ID NO:36. The amino acid sequence of full-length mouse IL-17F protein encoded by SEQ ID NO:36 is set forth as SEQ ID NO:37.

IL-17R nucleotide and amino acid sequences are known in the art and are provided. The nucleotide sequence of a cDNA encoding human IL-17R is set forth as SEQ ID NO:5, which includes a poly(A) tail. Nucleic acid residues 134-2734 represent the open reading frame of SEQ ID NO:5, which includes a stop codon. The amino acid sequence of full-length human IL-17R protein encoded by SEQ ID NO:5 is set forth as SEQ ID NO:6. An additional nucleic acid sequence for human IL-17R is provided by NCBI Accession No. BC011624, and is set forth as SEQ ID NO:28. SEQ ID NO:28 encodes an 866 amino acid protein, set forth as SEQ ID NO:29.

IL-17RC nucleotide and amino acid sequences are known in the art and are provided. The nucleotide sequences of several cDNAs encoding human IL-17RC are set forth as SEQ ID NOs:7, 9, 11, 13, and 15, which include a poly(A) tail. Nucleic acid residues 219-2594, 219-2381, 219-1835, 219-1022, and 219-494 represent the open reading frames of SEQ ID NOs:7, 9, 11, 13, and 15, respectively, which include stop codons. The amino acid sequences of full-length IL-17RC proteins encoded by SEQ ID NOs:7, 9, 11, 13, and 15 are set forth as SEQ ID NOs:8, 10, 12, 14, and 16, respectively. An additional nucleic acid sequence for human IL-17RC is provided by NCBI Accession No. AY359098, and is set forth as SEQ ID NO:26. SEQ ID NO:26 encodes a 705 amino acid protein, set forth as SEQ ID NO:27.

The nucleic acids related to the present invention may comprise DNA or RNA and may be wholly or partially synthetic. Reference to a nucleotide sequence as set out herein encompasses a DNA molecule with the specified sequence, and further encompasses an RNA molecule with the specified sequence or its complement, in which U is substituted for T, unless context requires otherwise.

The isolated polynucleotides related to the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridizations, in situ hybridization and Northern hybridization, and are well known to those skilled in the art.

Hybridization reactions may be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Stringency Conditions

| | | Hybrid | | Wash |
|---|---|---|---|---|
| Stringency | Polynucleotide | Length | Hybridization Temperature and | Temperature and |
| Condition | Hybrid | (bp)¹ | Buffer² | Buffer² |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4x SSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4x SSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2x SSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6x SSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6x SSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1 SSPE is 0.15M NaCl, 10 mM NaH₂PO₄, and 1.25 mM EDTA, pH 7.4) can be substituted for SSC (1 SSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀ Na⁺) + 0.41(% G + C) − (600/N), where N is the number of bases in the hybrid, and Na⁺ is the concentration of sodium ions in the hybridization buffer (Na⁺for 1xSSC = 0.165M).
Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, 1995, F. M. Ausubel et al., eds., John Wiley & Sons, Inc., sections 2.10 and 6.3-6.4, incorporated herein by reference.

The isolated polynucleotides related to the present invention may be used as hybridization probes and primers to identify and isolate DNA having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least 90% sequence identity (more preferably, at least 95% identity; most preferably, at least 99% identity) with the disclosed polynucleotides. Alternatively, significant similarity exists when the nucleic acid segments will hybridize under selective hybridization conditions (e.g., highly stringent hybridization conditions) to the disclosed polynucleotides.

The isolated polynucleotides related to the present invention may also be used as hybridization probes and primers to identify and isolate DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. Preferably, polynucleotide homologs have at least 50% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

Calculations of “homology” or “sequence identity” between two sequences may be performed as follows. The sequences may be aligned for optimal comparison purposes (e.g., gaps can be introduced in one or both of a first and a second amino acid or nucleic acid sequence for optimal alignment and nonhomologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions may then be compared. When a position in the first sequence is occupied by the same amino acid residue or nucleotide as the corresponding position in the second sequence, then the molecules are identical or have homology at that position. The percent identity between the two sequences is a function of the number of identical positions shared by the sequences, taking into account the number of gaps, and the length of each gap, which need to be introduced for optimal alignment of the two sequences.

The comparison of sequences and determination of percent sequence identity between two sequences may be accomplished using a mathematical algorithm. In one embodiment, the percent identity between two amino acid sequences is determined using the Needleman and Wunsch algorithm ((1970) J. Mol. Biol. 48:444-53), which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In another embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A preferred set of parameters (and the one that can be used if a practitioner is uncertain about which parameters should be applied to determine whether a molecule is within a sequence identity or homology limitation of the invention) is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17), which has been incorporated into the ALIGN program (version 2.0), using a PAM120 weight residue table, a gap length penalty of 12 and a gap penalty of 4.

The polynucleotides related to the present invention may also be used as hybridization probes and primers to identify cells and tissues that express the polypeptides related to the present invention and the conditions under which they are expressed.

Additionally, the function of the polypeptides related to the present invention may be directly examined by using the polynucleotides encoding the polypeptides to alter (i.e., enhance, reduce, or modify) the expression of the genes corresponding to the polynucleotides related to the present invention in a cell or organism. These “corresponding genes” are the genomic DNA sequences related to the present invention that are transcribed to produce the mRNAs from which the polynucleotides related to the present invention are derived.

Altered expression of the genes related to the present invention may be achieved in a cell or organism through the use of various inhibitory polynucleotides, such as antisense polynucleotides and ribozymes that bind and/or cleave the mRNA transcribed from the genes related to the invention (see, e.g., Galderisi et al. (1999) J. Cell Physiol. 181:251-57; Sioud (2001) Curr. Mol. Med. 1:575-88). An inhibitory polynucleotide(s), e.g., to IL-17F, IL-17A, IL-17R, and/or IL-17RC, may be used as an IL-17F/IL-17A antagonist (signaling antagonist), e.g., to inhibit IL-17F/IL-17A binding to its receptor (e.g., IL-17R and/or IL-17RC). Consequently, such inhibitory polynucleotides may be useful in preventing or treating IL-17F/IL-17A-associated disorders.

The antisense polynucleotides or ribozymes related to the invention may be complementary to an entire coding strand of a gene related to the invention, or to only a portion thereof. Alternatively, antisense polynucleotides or ribozymes can be complementary to a noncoding region of the coding strand of a gene related to the invention. The antisense polynucleotides or ribozymes can be constructed using chemical synthesis and enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Micklefield (2001) Curr. Med. Chem. 8:1157-79). Alternatively, these molecules can be produced biologically using an expression vector into which a polynucleotide related to the present invention has been subcloned in an antisense (i.e., reverse) orientation.

The inhibitory polynucleotides of the present invention also include triplex-forming oligonucleotides (TFOs) that bind in the major groove of duplex DNA with high specificity and affinity (Knauert and Glazer (2001) Hum. Mol. Genet. 10:2243-51). Expression of the genes related to the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nat. Med. 9:347-51). In addition, intrathecally delivered siRNA has recently been reported to block pain responses in two models (agonist-induced pain model and neuropathic pain model) in the rat (Dorn et al. (2004) Nucleic Acids Res. 32(5):e49).

The siRNA molecules of the present invention may be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules may be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to the polynucleotides related to the present invention can be designed based on criteria well known in the art (e.g., Elbashir et al. (2001) EMBO J. 20:6877-88). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; and the target segment preferably should be in the ORF region of the target mRNA and preferably should be at least 75 bp after the initiation ATG and at least 75 bp before the stop codon. Based on these criteria, or on other known criteria (e.g., Reynolds et al. (2004) Nat. Biotechnol. 22:326-30), siRNA molecules related to the present invention that target the mRNA polynucleotides related to the present invention may be designed by one of ordinary skill in the art.

Table 2 sets forth exemplary polynucleotide sequences on which to base siRNA molecules related to the invention, and an alternative sequence name, the SEQ ID NO, and target for each. As set forth in Table 2, the sequences set forth as SEQ ID NOs:17-20 represent polynucleotide sequences on which to base siRNA molecules for hIL-17R, and SEQ ID NOs:21-24 represent polynucleotide sequences on which to base siRNA molecules for hIL-17RC. The siRNA molecules based on the sequences set forth as SEQ ID NOs:17-20 were successfully used to target expression of hIL-17R, and siRNA molecules based on the sequences set forth as SEQ ID NOs:21-24 were successfully used to target the expression of hIL-17RC (see Example 1.2.6).

TABLE 2
Exemplary siRNA molecules

| | | Alternative | Polynucleotide |
|---|---|---|---|
| Target | SEQ ID NO | sequence name | sequence |
| IL-17R | SEQ ID NO:17 | R-1 | CAG CGG TCT GGT |
| | | | TAT CGT CTA |
| IL-17R | SEQ ID NO:18 | R-2 | CGG CAC CTA CGT |
| | | | AGT CTG CTA |
| IL-17R | SEQ ID NO:19 | R-3 | CAG GAA GGT CTG |
| | | | GAT CAT CTA |
| IL-17R | SEQ ID NO:20 | R-4 | CAG GTT TGA GTT |
| | | | TCT GTC CAA |
| IL-17RC | SEQ ID NO:21 | RC-1 | ACC GCA GAT CAT |
| | | | TAC CTT GAA |
| IL-17RC | SEQ ID NO:22 | RC-2 | CAG GTA CGA GAA |
| | | | GGA ACT CAA |
| IL-17RC | SEQ ID NO:23 | RC-3 | CGG GAC TTA AAT |
| | | | AAA GGC AGA |
| IL-17RC | SEQ ID NO:24 | RC-4 | CCG CGC GGC TCT |
| | | | GCT CCT CTA |

Inhibitory polynucleotides, e.g., siRNA, antisense polynucleotides, ribozymes, TFOs, etc., for IL-17F may target the expression of IL-17F and/or IL-17F/IL-17A. Similarly, inhibitory polynucleotides for IL-17A may target the expression of IL-17A and/or IL-17F/IL-17A. Further, treating a cell with inhibitory polynucleotides for either or both IL-17F and IL-17A may target the expression of the IL-17F/IL-17A heterodimer. Thus, inhibitory polynucleotides to either or both IL-17F and IL-17A may also be considered IL-17F/IL-17A signaling antagonists.

Altered expression of the genes related to the present invention in an organism may also be achieved through the creation of nonhuman transgenic animals into whose genomes polynucleotides related to the present invention have been introduced. Such transgenic animals include animals that have multiple copies of a gene (i.e., the transgene) of the present invention. A tissue-specific regulatory sequence(s) may be operably linked to the transgene to direct expression of a polypeptide related to the present invention to particular cells or a particular developmental stage. Methods for generating transgenic animals via embryo manipulation and microinjection, particularly animals such as mice, have become conventional and are well known in the art (e.g., Bockamp et al. (2002) Physiol. Genomics 11:115-32).

Altered expression of the genes related to the present invention in an organism may also be achieved through the creation of animals whose endogenous genes corresponding to the polynucleotides related to the present invention have been disrupted through insertion of extraneous polynucleotide sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al. (2002) Trends Neurosci. 25:336-40).

The isolated polynucleotides of the present invention also may be operably linked to an expression control sequence and/or ligated into an expression vector for recombinant production of the polypeptides (including active fragments and/or fusion polypeptides thereof) related to the present invention. General methods of expressing recombinant proteins are well known in the art.

An expression vector, as used herein, is intended to refer to a nucleic acid molecule capable of transporting another nucleic acid to which it has been linked. One type of vector is a plasmid, which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as recombinant expression vectors (or simply, expression vectors). In general, expression vectors of utility in recombinant DNA techniques are often in the form of plasmids. In the present specification, plasmid and vector may be used interchangeably as the plasmid is the most commonly used form of vector. However, the invention is intended to include other forms of expression vectors, such as viral vectors (e.g., replication defective retroviruses, adenoviruses and adeno-associated viruses) that serve equivalent functions.

In one embodiment, the polynucleotides related to the present invention are used to create recombinant IL-17F/IL-17A signaling agonists, e.g., those that can be identified based on the presence of at least one IL-17F/IL-17A “receptor binding motif.” As used herein, the term “receptor binding motif” includes amino acid sequences or residues that are important for binding of the cytokine to its requisite receptor. For example, an IL-17F/IL-17A agonist (signaling agonist) includes IL-17F/IL-17A and/or fragments thereof, e.g., IL-17R or IL-17RC binding fragments. In another embodiment, the polynucleotides related to the present invention are used to create antagonists of IL-17F, IL-17A, and/or IL-17F/IL-17A signaling (e.g., IL-17F, IL-17A, IL-17R, and/or IL-17RC inhibitory polynucleotides; soluble IL-17R and/or IL-17RC polypeptides (including fragments (e.g., IL-17F, IL-17A, and/or IL-17F/IL-17A binding fragments) and/or fusion proteins thereof); inhibitory anti-IL-17F, anti-IL-17A, anti-IL-17F/IL-17A, anti-IL-17R, and/or IL-17RC antibodies; antagonistic small molecules, etc.).

Methods of creating fusion polypeptides, i.e., a first polypeptide moiety linked with a second polypeptide moiety, are well known in the art. For example, a polypeptide related to the invention (e.g., IL-17A homodimer, IL-17F homodimer, IL-17F/IL-17A heterodimer, IL-17R, IL-17RC, and fragments thereof) may be fused to a second polypeptide moiety, e.g., an immunoglobulin or a fragment thereof (e.g., an Fc binding fragment thereof). In some embodiments, the first polypeptide moiety includes a full-length polypeptide related to the invention. Alternatively, the first polypeptide may comprise less than the full-length polypeptide. Additionally, a soluble form of a polypeptide related to the invention may be fused to the Fc portion of an immunoglobulin (see, e.g., Example 1.1.2) with or without a “linker” sequence linking the polypeptide related to the invention and the Fc portion of the immunoglobulin. Other fusions proteins, such as those with glutathione-S-transferase (GST), Lex-A, thioredoxin (TRX), biotin, or maltose-binding protein (MBP), may also be used.

The second polypeptide moiety is preferably soluble. In some embodiments, the second polypeptide moiety enhances the half-life, (e.g., the serum half-life) of the linked polypeptide. In some embodiments, the second polypeptide moiety includes a sequence that facilitates association of the fusion polypeptide with another IL-17A, IL-17F, IL-17RC or IL-17R polypeptide, or association of IL-17A and IL-17F to form a heterodimer. In some embodiments, the second polypeptide includes at least a region of an immunoglobulin polypeptide. Immunoglobulin fusion polypeptide are known in the art and are described in, e.g., U.S. Pat. Nos. 5,516,964; 5,225,538; 5,428,130; 5,514,582; 5,714,147; and 5,455,165, all of which are hereby incorporated by reference in their entireties. The fusion proteins may additionally include a linker sequence joining the first polypeptide moiety, e.g., IL-17F, IL-17A, IL-17F/IL-17A, IL-17R, or IL-17RC, including fragments thereof, to the second moiety. Use of such linker sequences are well known in the art. For example, the fusion protein can include a peptide linker, e.g., a peptide linker of about 2 to 20, more preferably less than 10, amino acids in length. In one embodiment, the peptide linker may be two amino acids in length.

In another embodiment, the recombinant protein includes a heterologous signal sequence (i.e., a polypeptide sequence that is not present in a polypeptide encoded by an IL-17F, IL-17A, IL-17R or IL-17RC nucleic acid) at its N-terminus. For example, a signal sequence from another protein may be fused with a polypeptide related to the present invention, including fragments and/or fusion proteins thereof. In certain host cells (e.g., mammalian host cells), expression and/or secretion of recombinant proteins can be increased through use of a heterologous signal sequence. For example, a signal peptide that may be included in the fusion protein is the melittin signal peptide MKFLVNVALVFMVVYISYIYA (SEQ ID NO:25).

A fusion protein related to the invention may be produced by standard recombinant DNA techniques. For example, DNA fragments coding for the different polypeptide sequences are ligated together in-frame in accordance with conventional techniques by employing, e.g., blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments may be carried out using anchor primers that give rise to complementary overhangs between two consecutive gene fragments that can subsequently be annealed and reamplified to generate a chimeric gene sequence (see, for example, Ausubel et al. (Eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). For example, an IL-17F-, IL-17A-, IL-17R- and/or IL-17RC-encoding nucleic acid may be cloned into such an expression vector such that the fusion moiety is linked in-frame to the immunoglobulin protein. In some embodiments, IL-17F, IL-17A, IL-17R and/or IL-17RC fusion polypeptides exist as oligomers, such as dimers, trimers, or tetramers. In one embodiment, IL-17F and IL-17A fusion polypeptides exist as heterodimers.

The recombinant expression vectors of the invention may carry additional sequences, such as sequences that regulate replication of the vector in host cells (e.g., origins of replication) and selectable marker genes. The selectable marker gene facilitates selection of host cells into which the vector has been introduced. For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Suitable vectors can be chosen or constructed, containing appropriate regulatory sequences, including promoter sequences, terminator sequences, polyadenylation sequences, enhancer sequences, marker genes and other sequences, e.g., sequences that regulate replication of the vector in the host cells (e.g., origins of replication) as appropriate. Vectors may be plasmids or viral, e.g., phage, or phagemid, as appropriate. For further details see, for example, Molecular Cloning: A Laboratory Manual: 2nd ed., Sambrook et al., Cold Spring Harbor Laboratory Press, 1989. Many known techniques and protocols for manipulation of nucleic acids, for example, in preparation of nucleic acid constructs, mutagenesis, sequencing, introduction of DNA into cells and gene expression, and analysis of proteins, are described in detail in Current Protocols in Molecular Biology, 2nd ed., Ausubel et al. eds., John Wiley & Sons, 1992.

Thus, a further aspect of the present invention provides a host cell comprising a nucleic acid as disclosed herein. A still further aspect provides a method comprising introducing such nucleic acid into a host cell. The introduction may employ any available technique. For eukaryotic cells, suitable techniques may include calcium phosphate transfection, DEAE-dextran, electroporation, liposome-mediated transfection, and transduction using retrovirus or other viruses, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. A number of cell lines may act as suitable host cells for recombinant expression of the polypeptides related to the present invention. Mammalian host cell lines include, for example, COS cells, CHO cells, 293 cells (e.g., HEK293 cells), A431 cells, 3T3 cells, CV-1 cells, HeLa cells, L cells, BHK21 cells, HL-60 cells, U937 cells, HaK cells, Jurkat cells, as well as cell strains derived from in vitro culture of primary tissue and primary explants. In one embodiment of the present invention, the IL-17F/IL-17A heterodimer may be produced by either simultaneously transfecting one cell with both IL-17F- and IL-17A-containing vectors, or transfecting one cell with a vector containing both IL-17F and IL-17A, and culturing the cell under conditions suitable for recombinant expression of both IL-17A and IL-17F, such that the IL-17F/IL-17A heterodimer is expressed.

Alternatively, it may be possible to recombinantly produce the polypeptides related to the present invention in lower eukaryotes, such as yeast, or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides related to the present invention are made in yeast or bacteria, it may be necessary to modify them by, for example, phosphorylation or glycosylation of appropriate sites, in order to obtain functionality. Such covalent attachments may be accomplished using well-known chemical or enzymatic methods.

Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent. When cysteine residues are present in the primary amino acid sequence of the protein, it is often necessary to accomplish the refolding in an environment that allows correct formation of disulfide bonds (a redox system). General methods of refolding are disclosed in Kohno (1990) Meth. Enzymol. 185:187-95. European Patent EP 0433225, and U.S. Pat. No. 5,399,677 describe other appropriate methods.

The polypeptides related to the present invention may also be recombinantly produced by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MaxBac® kit, Invitrogen, Carlsbad, Calif.).

Following recombinant expression in the appropriate host cells, the recombinant polypeptides of the present invention may then be purified from culture medium or cell extracts using known purification processes, such as gel filtration and ion exchange chromatography. For example, soluble forms of polypeptides related to the invention, e.g., IL-17F, IL-17A, IL-17F/IL-17A, IL-17R, IL-17RC proteins (including fragments, and/or fusion proteins thereof), antagonists thereof and agonists thereof may be purified from conditioned media. Membrane-bound forms of the polypeptides related to the invention may be purified by preparing a total membrane fraction from the expressing cell and extracting the membranes with a nonionic detergent such as Triton X-100. A polypeptide related to the present invention may be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) or polyethyleneimine (PEI) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred (e.g., S-SEPHAROSE® columns). The purification of recombinant proteins from culture supernatant may also include one or more column steps over such affinity resins as concanavalin A-agarose, heparin-TOYOPEARL® (Toyo Soda Manufacturing Co., Ltd., Japan) or Cibacron blue 3GA SEPHAROSE®; or by hydrophobic interaction chromatography using such resins as phenyl ether, butyl ether, or propyl ether; or by immunoaffinity chromatography. Finally, one or more reverse-phase high performance liquid chromatography (RP-HPLC) steps employing hydrophobic RP-HPLC media, e.g., silica gel having pendant methyl or other aliphatic groups, can be employed to further purify the recombinant protein. Affinity columns including antibodies (e.g., those described using the methods herein) to the recombinant protein may also be used in purification steps in accordance with known methods. Some or all of the foregoing purification steps, in various combinations or with other known methods, may also be employed to provide a substantially purified isolated recombinant protein. Preferably, the isolated recombinant protein is purified so that it is substantially free of other mammalian proteins. Additionally, these purification processes may also be used to purify the polypeptides of the present invention from other sources, including natural sources. For example, polypeptides related to the invention, which are expressed as a product of transgenic animals, e.g., as a component of the milk of transgenic cows, goats, pigs, or sheep, may be purified as described above.

Alternatively, the polypeptides may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen, respectively. Recombinant proteins can also be tagged with a small epitope and subsequently identified or purified using a specific antibody to the epitope. A preferred epitope is the FLAG epitope, which is commercially available from Eastman Kodak (New Haven, Conn.).

Alternatively, recombinant IL-17F and IL-17A fusion proteins may be tagged with different epitopes to allow purification of IL-17F/IL-17A heterodimers. The existence of different tags on IL-17F and IL-17A allows isolation of IL-17F/IL-17A heterodimers that are substantially free from both IL-17A and IL-17F homodimers. For example, IL-17A may be tagged with an epitope (e.g., FLAG, myc, etc., while IL-17F is concurrently tagged with a different epitope (e.g., His, GST, etc.) and both proteins simultaneously expressed in a cell. Extracts from the recombinant host cell, or media in which the host cells are cultured, can be obtained and subjected to two-step affinity chromatography purification under nonreducing conditions. The first affinity column would bind one of the two different tags, e.g., a FLAG epitope fused to IL-17A (or a fragment of IL-17A), and therefore the wash from the first column would contain (predominantly) IL-17F homodimers and the eluent from the first column would contain both IL-17F/IL-17A heterodimers and IL-17A homodimers. The eluent from the first column could then be placed over a second affinity column that specifically binds the other of the two different tags, e.g., a His tag fused to IL-17F. Thus, the wash from the second column would contain IL-17A homodimers and the eluent from the second column would be substantially free of both IL-17A and IL-17F homodimers (i.e., contain only IL-17F/IL-17A heterodimers). The extracts from the recombinant host cells or the host cell media could be obtained under nonreducing conditions such that protein-protein interactions are not interrupted, or could be obtained under reducing conditions and then treated to allow proper refolding and interactions of the IL-17F and IL-17A monomers contained therein. One skilled in the art will readily recognize that a host cell need not express both IL-17F and IL-17A fusion proteins; rather cell or media extracts from single transfectants, e.g., a host cell expressing either an IL-17A or an IL-17F fusion protein, could be obtained and combined under conditions that allow the IL-17A and IL-17F monomers to dimerize. Detailed methods of IL-17F/IL-17A heterodimer purification are described in U.S. patent application Ser. No. 11/353,161, incorporated by reference herein in its entirety.

The polypeptides related to the present invention may also be produced by known conventional chemical synthesis. Methods for chemically synthesizing such polypeptides are well known to those skilled in the art. Such chemically synthetic polypeptides may possess biological properties in common with the natural, purified polypeptides, and thus may be employed as biologically active or immunological substitutes for the natural polypeptides.

The polypeptides related to the present invention, including IL-17F, IL-17A, and IL-17F/IL-17A signaling agonists and antagonists, also encompass molecules that are structurally different from the disclosed polypeptides (e.g., which have a slightly altered sequence), but have substantially the same biochemical properties as the disclosed polypeptides (e.g., are changed only in functionally nonessential amino acid residues). Such molecules include naturally occurring allelic variants and deliberately engineered variants containing alterations, substitutions, replacements, insertions, or deletions. Techniques for such alterations, substitutions, replacements, insertions, or deletions are well known to those skilled in the art. In some embodiments, the polypeptide moiety is provided as a variant polypeptide having mutations in the naturally occurring sequence (e.g., wild type) that results in a sequence more resistant to proteolysis (relative to the nonmutated sequence).

The polypeptides according to the present invention can also include peptide mimetics. Peptide mimetics are peptide-containing molecules that mimic elements of protein secondary structure. See, for example, Johnson et al. “Peptide Turn Mimetics” in BIOTECHNOLOGY AND PHARMACY, Pezzuto et al., Eds., Chapman and Hall, New York (1993) (incorporated by reference herein in its entirety). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second-generation molecules having many of the natural properties of the disclosed targeting peptides or polypeptides, but with altered and even improved characteristics.

The polypeptides related to the invention may be used to screen agents (e.g., other IL-17F, IL-17A, and IL-17F/IL-17A signaling antagonists, e.g., anti-IL-17F, anti-IL-17A, and anti-IL-17F/IL-17A antibodies) that are capable of binding IL-17F/IL-17A and/or inhibiting IL-17F/IL-17A biological activity. Binding assays utilizing a desired binding protein, immobilized or not, are well known in the art and may be used for this purpose with the polypeptides related to the present invention. Purified cell-based or protein-based (cell-free) screening assays may be used to identify such agents. For example, IL-17F/IL-17A protein may be immobilized in purified form on a carrier, and binding of potential ligands to purified IL-17F/IL-17A may be measured.

### Antibodies

In some embodiments, the invention provides specific anti-IL-17F/IL-17A antibodies, i.e., intact antibodies or antigen binding fragments thereof, that bind to IL-17F/IL-17A heterodimer only. In another embodiment the invention provides selective anti-IL-17F/IL-17A antibodies that bind both IL-17F/IL-17A and one of IL-17F or IL-17A due to the selective antibody recognizing an epitope not specific to the IL-17F/IL-17A heterodimer but rather an epitope specific to IL-17F or IL-17A. In one embodiment, the antibodies are signaling antagonists (including specific and selective antagonists to IL-17F/IL-17A signaling), i.e., they inhibit at least one IL-17F/IL-17A biological activity (e.g., binding of the heterodimer to its receptor, heterodimer-mediated activation of signaling components, heterodimer-mediated induction of cytokine production (e.g., GRO-α), heterodimer induction of airway inflammation, etc.). The antagonistic antibodies of the invention may also be useful in diagnosing, prognosing, monitoring and/or treating IL-17F/IL-17A-associated disorders. A skilled artisan will recognize that selective and antagonistic IL-17F/IL-17A antibodies may inhibit at least one biological activity of both IL-17F/IL-17A and one of IL-17F or IL-17A. In another embodiment, the antibodies (including specific and selective antibodies) are detecting antibodies that specifically bind to but do not inhibit IL-17F/IL-17A signaling, and may be used to detect the presence of IL-17F/IL-17A, e.g., as part of a kit for diagnosing, prognosing, and/or monitoring a disorder(s) related to IL-17F/IL-17A signaling. In one embodiment, the antibody is a monoclonal antibody. The antibodies may also be human, humanized, chimeric, or in vitro-generated antibodies against human IL-17A, IL-17F, and/or IL-17F/IL-17A. In a preferred embodiment, the antibodies of the invention, e.g., antagonist antibodies or detecting antibodies, are directed toward mammalian, e.g., human IL-17F/IL-17A.

One of skill in the art will recognize that, as used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDRs”), interspersed with regions that are more conserved, termed “framework regions” (“FR”). The extent of the FRs and CDRs has been precisely defined (see, Kabat et al. (1991) Sequences of Proteins of Immunological Interest, Fifth Edition, U.S. Department of Health and Human Services, NIH Publication No. 91-3242; and Chothia et al. (1987) J. Mol. Biol. 196:901-17, which are hereby incorporated by reference herein in their entireties). Each VH and VL is composed of three CDRs and four FRs, arranged from amino-terminus to carboxy-terminus in the following order: FR1, CDR1, FR2, CDR2, FR3, CDR3, FR4.

The antibody may further include a heavy and light chain constant region to thereby form a heavy and light immunoglobulin chain, respectively. In one embodiment, the antibody is a tetramer of two heavy immunoglobulin chains and two light immunoglobulin chains, wherein the heavy and light immunoglobulin chains are interconnected, e.g., by disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

Immunoglobulin refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd, or 214 amino acids) are encoded by a variable region gene at the NH₂-terminus (about 110 amino acids) and a kappa or lambda constant region gene at the COOH-terminus. Full-length immunoglobulin “heavy chains” (about 50 Kd, or 446 amino acids) are similarly encoded by a variable region gene (about 116 amino acids) and one of the other aforementioned constant region genes, e.g., gamma (encoding about 330 amino acids). The immunoglobulin heavy chain constant region genes encode for the antibody class, i.e., isotype (e.g., IgM or IgG1). The antigen binding fragment of an antibody (or simply “antibody portion,” or “fragment”), as used herein, refers to one or more fragments of a full-length antibody that retain the ability to specifically bind to an antigen (e.g., CD3). Examples of binding fragments encompassed within the term “antigen binding fragment” of an antibody include (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al. (1989) Nature 341:544-46), which consists of a VH domain; and (vi) an isolated complementarity determining region (CDR), or a set of CDRs, e.g., two or three CDRs. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-26; and Huston et al. (1988) Proc. Natl. Acad. Sci. USA 85:5879-83). Such single chain antibodies are also intended to be encompassed within the term “antigen binding fragment” of an antibody. These antibody fragments are obtained using conventional techniques known to those skilled in the art, and the fragments are screened for utility in the same manner as are intact antibodies.

In some embodiments, the invention provides single domain antibodies. Single domain antibodies can include antibodies whose CDRs are part of a single domain polypeptide. Examples include, but are not limited to, heavy chain antibodies, antibodies naturally devoid of light chains, single domain antibodies derived from conventional four-chain antibodies, engineered antibodies and single domain scaffolds other than those derived from antibodies. Single domain antibodies may be any of those known in the art, or any future single domain antibodies. Single domain antibodies may be derived from any species including, but not limited to, mouse, human, camel, llama, goat, rabbit, bovine. According to one aspect of the invention, a single domain antibody as used herein is a naturally occurring single domain antibody known as heavy chain antibody devoid of light chains. Such single domain antibodies are disclosed in, e.g., WO 94/04678. This variable domain derived from a heavy chain antibody naturally devoid of light chain is known herein as a VHH or nanobody, to distinguish it from the conventional VH of four-chain immunoglobulins. Such a VHH molecule can be derived from antibodies raised in Camelidae species, for example in camel, llama, dromedary, alpaca and guanaco. Other species besides those in the family Camelidae may produce heavy chain antibodies naturally devoid of light chain; such VHH molecules are within the scope of the invention.

Antibody molecules to the polypeptides of the present invention may be produced by methods well known to those skilled in the art. For example, monoclonal antibodies may be produced by generation of hybridomas in accordance with known methods. Hybridomas formed in this manner are then screened using standard methods, such as an enzyme-linked immunosorbent assay (ELISA), to identify one or more hybridomas that produce an antibody that specifically binds with the polypeptides of the present invention. For example, IL-17F/IL-17A may be used to immunize animals to obtain polyclonal and monoclonal antibodies that bind the IL-17F/IL-17A heterodimer specifically (i.e., do not bind either IL-17F or IL-17A) or selectively (i.e., bind to both IL-17F/IL-17A and either IL-17F or IL-17A (or both)). Similarly, IL-17R or IL-17RC proteins may be used to obtain polyclonal and monoclonal antibodies that react with IL-17R or IL-117RC, respectively, and that may inhibit binding of these receptors to IL-17F/IL-17A only, or both IL-17F/IL-17A and either one of IL-17F or IL-17A. IL-17R or IL-17RC proteins may also be used to obtain polyclonal and monoclonal antibodies that specifically react with IL-17R or IL-17RC, respectively, and which may inhibit binding of these receptors to any of IL-17A, IL-17F, and/or IL-17F/IL-17A cytokines. The peptide immunogens additionally may contain a cysteine residue at the carboxyl terminus, and may be conjugated to a hapten such as keyhole limpet hemocyanin (KLH). Additional peptide immunogens may be generated by replacing tyrosine residues with sulfated tyrosine residues. Methods for synthesizing such peptides are well known in the art, for example, as in Merrifield (1963) J. Amer. Chem. Soc. 85:2149-54; Krstenansky et al. (1987) FEBS Lett. 211:10-16. A full-length polypeptide of the present invention may be used as the immunogen, or, alternatively, antigenic peptide fragments of the polypeptides may be used. An antigenic peptide of a polypeptide of the present invention comprises at least seven continuous amino acid residues and encompasses an epitope such that an antibody raised against the peptide forms a specific immune complex with the polypeptide. Preferably, the antigenic peptide comprises at least 10 amino acid residues, more preferably at least 15 amino acid residues, even more preferably at least 20 amino acid residues, and most preferably at least 30 amino acid residues.

Monoclonal antibodies may be generated by other methods known to those skilled in the art of recombinant DNA technology. As an alternative to preparing monoclonal antibody-secreting hybridomas, a monoclonal antibody to a polypeptide of the present invention may be identified and isolated by screening a recombinant combinatorial immunoglobulin library (e.g., an antibody phage display library) with a polypeptide related to the present invention to thereby isolate immunoglobulin library members that bind to the polypeptides related to the present invention. Techniques and commercially available kits for generating and screening phage display libraries are well known to those skilled in the art. Additionally, examples of methods and reagents particularly amenable for use in generating and screening antibody display libraries can be found in the literature. For example, the “combinatorial antibody display” method is well known and was developed to identify and isolate antibody fragments having a particular antigen specificity, and can be utilized to produce monoclonal antibodies (for descriptions of combinatorial antibody display, see, e.g., Sastry et al. (1989) Proc. Natl. Acad. Sci. USA 86:5728; Huse et al. (1989) Science 246:1275; Orlandi et al. (1989) Proc. Natl. Acad. Sci. USA 86:3833). After immunizing an animal with an immunogen as described above, the antibody repertoire of the resulting B cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primers to a conserved 3′ constant region, can be used for PCR amplification of the heavy and light chain variable regions from a number of mouse antibodies (Larrick et al. (1991) Biotechniques 11:152-56). A similar strategy can also been used to amplify human heavy and light chain variable regions from human antibodies (Larrick et al. (1991) Methods: Companion to Methods in Enzymology 2:106-10).

Polyclonal sera and antibodies may be produced by immunizing a suitable subject with a polypeptide related to the present invention. The antibody titer in the immunized subject may be monitored over time by standard techniques, such as with ELISA using immobilized protein. If desired, the antibody molecules directed against a polypeptide of the present invention may be isolated from the subject or culture media and further purified by well-known techniques, such as protein A chromatography, to obtain an IgG fraction.

Fragments of antibodies to the polypeptides of the present invention may be produced by cleavage of the antibodies in accordance with methods well known in the art. For example, immunologically active Fab and F(ab′)₂ fragments may be generated by treating the antibodies with an enzyme such as pepsin.

Additionally, chimeric, humanized, and single-chain antibodies to the polypeptides of the present invention, comprising both human and nonhuman portions, may be produced using standard recombinant DNA techniques and/or a recombinant combinatorial immunoglobulin library. Humanized antibodies may also be produced using transgenic mice which are incapable of expressing endogenous immunoglobulin heavy and light chain genes, but which can express human heavy and light chain genes. For example, human monoclonal antibodies (mAbs) directed against, e.g., IL-17F/IL-17A, may be generated using transgenic mice carrying the human immunoglobulin genes rather than mouse immunoglobulin genes. Splenocytes from these transgenic mice immunized with the antigen of interest may then be used to produce hybridomas that secrete human mAbs with specific affinities for epitopes from a human protein (see, e.g., Wood et al., WO 91/00906; Kucherlapati et al., WO 91/10741; Lonberg et al. WO 92/03918; Kay et al., WO 92/03917; Lonberg et al. (1994) Nature 368:856-59; Green et al. (1994) Nat. Genet. 7:13-21; Morrison et al. (1994) Proc. Natl. Acad. Sci. USA 81:6851-55; Bruggeman (1993) Year Immunol. 7:33-40; Tuaillon et al. (1993) Proc. Natl. Acad. Sci. USA 90:3720-24; Bruggeman et al. (1991) Eur. J. Immunol. 21:1323-26).

Chimeric antibodies, including chimeric immunoglobulin chains, may be produced by recombinant DNA techniques known in the art. For example, a gene encoding the Fc constant region of a mouse (or other species) monoclonal antibody molecule is digested with restriction enzymes to remove the region encoding the mouse Fc, and the equivalent portion of a gene encoding a human Fc constant region is substituted (see Robinson et al., International Patent Publication PCT/US86/02269; Akira et al., European Patent Application EP 184,187; Taniguchi, European Patent Application EP 171,496; Morrison et al., European Patent Application EP 173,494; Neuberger et al., WO 86/01533; Cabilly et al., U.S. Pat. No. 4,816,567; Cabilly et al., European Patent Application EP 125,023; Better et al. (1988) Science 240:1041-43; Liu et al. (1987) Proc. Natl. Acad. Sci. USA 84:3439-43; Liu et al. (1987) J. Immunol. 139:3521-26; Sun et al. (1987) Proc. Natl. Acad. Sci. USA 84:214-18; Nishimura et al. (1987) Cancer Res. 47:999-1005; Wood et al. (1985) Nature 314:446-49; and Shaw et al. (1988) J. Natl. Cancer Inst. 80:1553-59).

An antibody or an immunoglobulin chain may be humanized by methods known in the art. Humanized antibodies, including humanized immunoglobulin chains, may be generated by replacing sequences of the Fv variable region that are not directly involved in antigen binding with equivalent sequences from human Fv variable regions. General methods for generating humanized antibodies are provided by Morrison (1985) Science 229:1202-07; Oi et al. (1986) BioTechniques 4:214; Queen et al., U.S. Pat. Nos. 5,585,089; 5,693,761; 5,693,762, the entire contents of all of which are hereby incorporated by reference herein. Those methods include isolating, manipulating, and expressing the nucleic acid sequences that encode all or part of immunoglobulin Fv variable regions from at least one of a heavy or light chain. Sources of such nucleic acid sequences are well known to those skilled in the art and, for example, may be obtained from a hybridoma producing an antibody against a predetermined target. The recombinant DNA encoding the humanized antibody, or fragment thereof, can then be cloned into an appropriate expression vector.

Humanized or CDR-grafted antibody molecules or immunoglobulins may be produced by CDR grafting or CDR substitution, wherein one, two, or all CDRs of an immunoglobulin chain can be replaced. See, e.g., U.S. Pat. No. 5,225,539; Jones et al. (1986) Nature 321:552-25; Verhoeyan et al. (1988) Science 239:1534; Beidler et al. (1988) J. Immunol. 141:4053-60; Winter, U.S. Pat. No. 5,225,539, the entire contents of all of which are hereby incorporated by reference herein. Winter describes a CDR-grafting method that may be used to prepare the humanized antibodies of the present invention (UK Patent Application GB 2188638A; Winter, U.S. Pat. No. 5,225,539), the entire contents of which are hereby incorporated by reference herein. All of the CDRs of a particular human antibody may be replaced with at least a portion of a nonhuman CDR, or only some of the CDRs may be replaced with nonhuman CDRs. It is only necessary to replace the number of CDRs required for binding of the humanized antibody to a predetermined antigen.

Monoclonal, chimeric and humanized antibodies that have been modified by, e.g., deleting, adding, or substituting other portions of the antibody, e.g., the constant region, are also within the scope of the invention. As nonlimiting examples, an antibody can be modified by deleting the constant region, by replacing the constant region with another constant region, e.g., a constant region meant to increase half-life, stability, or affinity of the antibody, or a constant region from another species or antibody class, and by modifying one or more amino acids in the constant region to alter, for example, the number of glycosylation sites, effector cell function, Fc receptor (FcR) binding, complement fixation, etc.

Methods for altering an antibody constant region are known in the art. Antibodies with altered function, e.g. altered affinity for an effector ligand, such as FcR on a cell, or the C1 component of complement, can be produced by replacing at least one amino acid residue in the constant portion of the antibody with a different residue (see, e.g., EP 388151A1, U.S. Pat. No. 5,624,821 and U.S. Pat. No. 5,648,260, the entire contents of all of which are hereby incorporated by reference herein). Similar types of alterations to the mouse (or other species) immunoglobulin may be applied to reduce or eliminate these functions; such alterations are known in the art.

For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or an aromatic nonpolar residue such as phenylalanine, tyrosine, tryptophan or alanine (see, e.g., U.S. Pat. No. 5,624,821).

The antibodies of the invention may be useful for isolating, purifying, and/or detecting the polypeptides of the invention in supernatant, cellular lysate, or on the cell surface. In one embodiment, an anti-IL-17F/IL-17A antibody is used to isolate, purify, and/or detect IL-17F/IL-17A. In another embodiment, anti-IL-17A and anti-IL-17F antibodies can isolate, purify, and/or detect only IL-17A and IL-17F, respectively. In yet another embodiment, anti-IL-17A and anti-IL-17F antibodies can also isolate, purify, and/or detect the IL-17F/IL-17A heterodimer. Antibodies disclosed in this invention may be also used diagnostically to monitor, e.g., IL-17F/IL-17A protein levels, as part of a clinical testing procedure, or clinically to target a therapeutic modulator to a cell or tissue comprising the antigen of the antibody. For example, a therapeutic such as a small molecule, or other therapeutic of the invention may be linked to an antibody of the invention in order to target the therapeutic to the cell or tissue expressing the polypeptide of the invention. Antagonistic antibodies (preferably monoclonal antibodies) that bind to IL-17F, IL-17A, IL-17F/IL-17A, IL-17R, or IL-17RC protein may also be useful in the treatment of a disease(s) related to IL-17F/IL-17A signaling. Thus, the present invention further provides compositions comprising a specific antagonistic IL-17F/IL-17A antibody, i.e., an antibody that specifically binds to IL-17F/IL-17A and decreases, limits, blocks, or otherwise reduces IL-17F/IL-17A signaling. The present invention also provides compositions comprising a signaling antagonist that decreases the signaling of any of IL-17F, IL-17A, and IL-17F/IL-17A, and thus reduces the signaling downstream of all three cytokines. Similarly, anti-IL-17F, anti-IL-17A, anti-IL17F/IL-17A, anti-IL-17R, or anti-IL-17RC antibodies may be useful in isolating, purifying, detecting, and/or diagnostically monitoring IL-17F, IL-17A, IL-17F/IL-17A, IL-17R, or IL-117RC, respectively, and/or clinically targeting a therapeutic modulator to a cell or tissue comprising IL-17F, IL-17A, IL-17F/IL-17A, IL-17R, or IL-17RC, respectively. Anti-IL-17F and anti-IL-17A antibodies can also be useful in isolating, purifying, detecting, and/or diagnostically monitoring IL-17F/IL-17A, or clinically monitoring a therapeutic modulator to a cell or tissue comprising IL-17F/IL-17A.

In addition to antibodies for use in the present invention, other molecules may also be employed to modulate the activity of IL-17F homodimers, IL-17A homodimers, and/or IL-17F/IL-17A heterodimers. Such molecules include small modular immunopharmaceutical (SMIP™) drugs (Trubion Pharmaceuticals, Seattle, Wash.). SMIPs are single-chain polypeptides composed of a binding domain for a cognate structure such as an antigen, a counterreceptor or the like, a hinge-region polypeptide having either one or no cysteine residues, and immunoglobulin CH2 and CH3 domains (see also www.trubion.com). SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

### Screening Assays

The polynucleotides and polypeptides related to the invention may be used in screening assays to identify pharmacological agents or lead compounds for agents that are capable of modulating the biological activity of IL-17F/IL-17A in a cell or organism and are thereby potential regulators of inflammatory responses. For example, samples containing at least IL-17F/IL-17A may be contacted with one of a plurality of test compounds (either biological agents or small organic molecules), and the biological activity of IL-17F/IL-17A (e.g., binding of the IL-17F/IL-17A to either or both IL-17R and IL-17RC, IL-17F/IL-17A-associated airway inflammation (e.g., neutrophil recruitment, cytokine production etc.)) in each of the treated samples can be compared with the biological activity of IL-17F/IL-17A in untreated samples or in samples contacted with different test compounds. Such comparisons will determine whether any of the test compounds results in: 1) a substantially decreased level of expression or biological activity of IL-17F/IL-17A, thereby indicating an IL-17F/IL-17A antagonist, or 2) a substantially increased level of expression or biological activity of IL-17F/IL-17A, thereby indicating an IL-17F/IL-17A agonist. In one embodiment, the identification of IL-17F/IL-17A modulators (e.g., test compounds capable of modulating IL-17F/IL-17A activity) is performed using high-throughput screening assays, such as BIACORE® (Biacore International AB, Uppsala, Sweden), BRET (bioluminescence resonance energy transfer), and FRET (fluorescence resonance energy transfer) assays, as well as ELISA and cell-based assays.

One skilled in the art will recognize that pharmacological agents or lead compounds that are capable of modulating the biological activity of IL-17F/IL-17A may also be capable of modulating the biological activity of either IL-17F and/or IL-17A. Thus, the present invention also provides methods of identifying whether an IL-17F/IL-17A modulator (e.g., an IL-17F/IL-17A signaling agonist or an IL-17F/IL-17A signaling antagonist) is a specific IL-17F/IL-17A modulator (i.e., it modulates the biological activity of IL-17F/IL-17A only), or a selective IL-17F/IL-17A modulator (i.e., it modulates the biological activity of both IL-17F/IL-17A and either one or both of IL-17A and IL-17F). For example, a compound that may modulate the biological activity of IL-17F/IL-17A, e.g., a compound capable of modulating the interaction of IL-17F/IL-17A to either or both IL-17R and IL-17RC, a compound capable of modulating IL-17F/IL-17A-associated airway inflammation (e.g., neutrophil recruitment, cytokine production etc.), etc., may be contacted with a sample containing at least IL-17A, and the biological activity of the IL-17A, e.g., binding of IL-17A to IL-17R and/or IL-17RC, or IL-17A-associated airway inflammation (e.g., neutrophil recruitment, cytokine production etc.), etc., in the treated sample can be compared with the biological activity of IL-17A in the untreated sample.

The compound may also be contacted with a sample containing at least IL-17F, and the biological activity of the IL-17F, e.g., binding of IL-17F to IL-17R and/or IL-17RC, or IL-17F-associated airway inflammation (e.g., neutrophil recruitment, cytokine production etc.), etc., in the treated sample can be compared to the biological activity of IL-17F in the untreated sample. Modulation of IL-17A and/or IL-17F biological activity (i.e., an increase or decrease in biological activity) will indicate that the IL-17F/IL-17A modulator is not a specific IL-17F/IL-17A modulator, but rather may be a selective IL-17F/IL-17A modulator. On the other hand, failure of the compound to modulate the biological activities of both IL-17A and IL-17F will indicate that the IL-17F/IL-17A modulator is a specific IL-17F/IL-17A modulator. An ordinarily skilled artisan will recognize that the steps of identifying whether a test compound is an IL-17A modulator [e.g., the steps of contacting a sample containing IL-17A and either or both IL-17R and IL-17RC with the test compound and determining whether the biological activity of IL-17A in the sample is modulated (e.g., increased or decreased) relative to the biological activity of IL-17A in a sample not contacted with the test compound] and identifying whether an test compound is an IL-17F modulator [e.g., the steps of contacting a sample containing IL-17F and either or both IL-17R and IL-17RC with the test compound and determining whether the biological activity of IL-17F in the sample is modulated (e.g., increased or decreased) relative to the biological activity of IL-17F in a sample not contacted with the test compound] may be performed sequentially in any order or simultaneously, and may be performed before, after, or simultaneously with methods of identifying whether the test compound is capable of modulating the biological activity of IL-17F/IL-17A [e.g., comprising the steps of contacting a sample containing IL-17F/IL-17A and either or both IL-17R and IL-17RC with a test compound and determining whether the biological activity of IL-17F/IL-17A in the sample is increased or decreased relative to the biological activity of IL-17F/IL-17A in a sample not contacted with the test compound, whereby such an increase or decrease in the biological activity of IL-17F/IL-17A in the sample contacted with the test compound identifies the compound as an IL 17F/IL 17A modulator (e.g., an IL-17F/IL-17A signaling agonist or an IL-17F/IL-17A signaling antagonist)].

In another embodiment, the identification of IL-17F/IL-17A modulators, including specific IL-17F/IL-17A modulators (e.g., specific IL-17F/IL-17A antagonists), is performed using a mouse model of airway inflammation, e.g., as described in Examples 2.1.6 and 2.2.4. For example, an experimental subject suffering from airway inflammation (e.g., a mouse into which ovalbumin-reactive Th17 cells have been adoptively transferred and which has been challenged with ovalbumin, a mouse that has been subjected to a dose of IL-17F/IL-17A, IL-17A, or IL-17F (e.g., intranasally)) may be treated with one of a plurality of test compounds (e.g., either biological agents or small organic molecules), and the level of airway inflammation (e.g., neutrophil recruitment, inflammatory cytokine concentration) in each of the treated subjects can be compared with the level of airway inflammation in untreated subjects or in subjects contacted with different test compounds. Such comparisons will determine whether any of the test compounds results in: 1) a substantially decreased level of airway inflammation, thereby indicating an IL-17F/IL-17A antagonist, or 2) a substantially increased level of airway inflammation, thereby indicating an IL-17F/IL-17A agonist. A skilled artisan will recognize that a test compound that (1) modulates IL-17F/IL-17A-associated airway inflammation (e.g., in a mouse subjected to a dose of IL-17F/IL-17A), (2) does not modulate IL-17A-associated airway inflammation (e.g., in a mouse subjected to a dose of IL-17A), and (3) does not modulate IL-17F-associated airway inflammation (e.g., in a mouse subjected to a dose of IL-17F) is a specific IL-17F/IL-17A modulator.

### Small Molecules

Decreased IL-17A, IL-17F, and/or IL-17F/IL-17A biological activity in an organism (or subject) afflicted with (or at risk for) disorders related to IL-17F/IL-17A signaling (e.g., IL-17F/IL-17A-associated disorders), or in a cell from such an organism (or subject) involved in such disorders, may also be achieved through the use of small molecules (usually organic small molecules) that antagonize, i.e., inhibit the activity of, IL-17F/IL-17A. Novel antagonistic small molecules may be identified by the screening methods described herein and may be used in the treatment methods of the present invention described below.

Conversely, increased IL-17F/IL-17A activity in an organism (or subject) afflicted with (or at risk for), e.g., an immune deficiency, e.g., neutropenia, or in a cell from such an organism (or subject) involved in such a disorder, may also be achieved through the use of small molecules (usually organic small molecules) that agonize, i.e., enhance the activity of, IL-17F/IL-17A. Novel agonistic small molecules may be identified by the screening methods described herein and may be used in the methods of treating immune deficiencies, e.g., as described in U.S. Pat. Nos. 5,707,829; 6,043,344; 6,074,849 and U.S. patent application Ser. No. 10/102,080, all of which are incorporated by reference herein in their entireties.

In some embodiments of the invention, an antagonistic or agonistic small molecule may be specific for IL-17F/IL-17A heterodimer (i.e., a small molecule binds and modulates the biological activity of the heterodimer only). One skilled in the art would recognize that specific IL-17F/IL-17A antagonistic or agonistic small molecules will be useful in respectively decreasing or increasing the activity of IL-17F/IL-17A only, and thus will be useful in treatment of IL-17F/IL-17A-associated diseases (i.e., diseases where a subject has altered IL-17F/IL-17A biological activity compared to IL-17F/IL-17A biological activity in a normal subject). In other embodiments of the invention, an antagonistic or an agonistic small molecule may be selective for IL-17F/IL-17A heterodimer (i.e., a small molecule that binds/modulates the biological activity of both IL-17F/IL-17A and either IL-17A or IL-17F (or both)). One skilled in the art would recognize that selective IL-17F/IL-17A antagonistic or agonistic small molecules will be useful in decreasing or increasing the activity of IL-17F/IL-17A and, e.g., either one of IL-17F or IL-17A, and thus will be useful in treatment of IL-17F/IL-17A-, IL-17A- and/or IL-17F-associated diseases/disorders (see U.S. patent application Ser. No. 11/353,161, incorporated herein by reference).

The term small molecule refers to compounds that are not macromolecules (see, e.g., Karp (2000) Bioinformatics Ontology 16:269-85; Verkman (2004) AJP-Cell Physiol. 286:465-74). Thus, small molecules are often considered those compounds that are, e.g., less than one thousand daltons (e.g., Voet and Voet, Biochemistry, 2^{nd} ed., ed. N. Rose, Wiley and Sons, New York, 14 (1995)). For example, Davis et al. (2005) Proc. Natl. Acad. Sci. USA 102:5981-86, use the phrase small molecule to indicate folates, methotrexate, and neuropeptides, while Halpin and Harbury (2004) PLos Biology 2:1022-30, use the phrase to indicate small molecule gene products, e.g., DNAs, RNAs and peptides. Examples of natural small molecules include, but are not limited to, cholesterols, neurotransmitters, and siRNAs; synthesized small molecules include, but are not limited to, various chemicals listed in numerous commercially available small molecule databases, e.g., FCD (Fine Chemicals Database), SMID (Small Molecule Interaction Database), ChEBI (Chemical Entities of Biological Interest), and CSD (Cambridge Structural Database) (see, e.g., Alfarano et al. (2005) Nuc. Acids Res. Database Issue 33:D416-24). Methods for Diagnosing, Prognosing, and Monitoring the Progress of Disorders Related to IL-17F/IL-17A Signaling

The present invention provides methods for diagnosing, prognosing, and monitoring the progress of IL-17F/IL-17A-associated disorders in a subject (e.g., disorders that directly or indirectly involve increases in the biological activity of IL-17F/IL-17A) by detecting an upregulation of IL-17F/IL-17A activity, e.g., by detecting the upregulation of IL-17F/IL-17A, including but not limited to the use of such methods in human subjects. A skilled artisan will recognize that disorders related to IL-17F/IL-17A may also be related to IL-17A and/or IL-17F biological activity. Thus, these methods may be performed by utilizing, e.g., prepackaged diagnostic kits comprising at least one of the group comprising one or more IL-17F, IL-17A, IL-17R, or IL-17RC polynucleotide(s) or fragment(s) thereof, one or more IL-17F, IL-17A, IL-17F/IL-17A, IL-17R, or IL-17RC polypeptide(s) or fragment(s) thereof (including fusion proteins thereof), one or more antibodies to an IL-17F, IL-17A, IL-17F/IL-17A, IL-17R, or IL-17RC polypeptide(s) or derivative(s) thereof, or one or more modulator(s) of IL-17F, IL-17A, IL-17F/IL-17A, IL-17R, or IL-17RC polynucleotide(s) and/or polypeptide(s) as described herein, which may be conveniently used, for example, in a clinical setting. In addition, one of skill in the art would recognize that the upregulation of, e.g., IL-17F/IL-17A, could also be detected by indirect methods, such as counting the number of immune cells, e.g., neutrophils.

“Diagnostic” or “diagnosing” means identifying the presence or absence of a pathologic condition. Diagnostic methods include detecting upregulation of IL-17F/IL-17A signaling by determining a test amount of IL-17F/IL-17A gene product(s) (e.g., mRNA, cDNA, and/or polypeptide, including fragments thereof) of IL-17F, IL-17A and/or IL-17F/IL-17A in a biological sample from a subject (e.g., a human or nonhuman mammal)), and comparing the test amount with a normal amount or range (i.e., an amount or range from an individual(s) known not to suffer from disorders related to IL-17F/IL-17A signaling). Although a particular diagnostic method may not provide a definitive diagnosis of disorders related IL-17F/IL-17A signaling, it suffices if the method provides a positive indication that aids in diagnosis.

The present invention also provides methods for prognosing such disorders by detecting the upregulation of IL-17F/IL-17A activity, e.g., by detecting upregulation of IL-17F/IL-17A. “Prognostic” or “prognosing” means predicting the probable development and/or severity of a pathologic condition. Prognostic methods include determining the test amount of a gene product(s) of IL-17F/IL-17A in a biological sample from a subject, and comparing the test amount to a prognostic amount or range (i.e., an amount or range from individuals with varying severities of IL-17F/IL-17A-associated disorders) for the gene product of IL-17F/IL-17A. Various amounts of the IL-17F/IL-17A gene product in a test sample are consistent with certain prognoses for disorders related to IL-17F/IL-17A signaling. The detection of an amount of IL-17F/IL-17A gene product at a particular prognostic level provides a prognosis for the subject.

The present invention also provides methods for monitoring the progress or course of such disorders related to IL-17F/IL-17A signaling by detecting the upregulation of IL-17F/IL-17A biological cytokine activity, e.g., by detecting upregulation of IL-17F/IL-17A gene products. Monitoring methods include determining the test amounts of a gene product of IL-17F/IL-17A in biological samples taken from a subject, e.g., at a first and second time, and comparing the amounts. A change in amount of an IL-17F/IL-17A gene product between the first and second times indicates a change in the course of IL-17F/IL-17A-associated disorders, with a decrease in amount indicating remission of such disorders, and an increase in amount indicating progression of such disorders. Such monitoring assays are also useful for evaluating the efficacy of a particular therapeutic intervention in patients being treated for, e.g., autoimmune disorders.

Increased IL-17F/IL-17A signaling in methods outlined above may be detected in a variety of biological samples, including bodily fluids (e.g., whole blood, plasma, and urine), cells (e.g., whole cells, cell fractions, and cell extracts), and other tissues. Biological samples also include sections of tissue, such as biopsies and frozen sections taken for histological purposes. Preferred biological samples include blood, plasma, lymph, tissue biopsies, urine, CSF (cerebrospinal fluid), synovial fluid, and BAL (bronchoalveolar lavage). It will be appreciated that analysis of a biological sample need not necessarily require removal of cells or tissue from the subject. For example, appropriately labeled agents that bind IL-17F/IL-17A signaling gene products (e.g., antibodies, nucleic acids) can be administered to a subject and visualized (when bound to the target) using standard imaging technology (e.g., CAT, NMR (MRI), and PET).

In the diagnostic and prognostic assays of the present invention, the IL-17F/IL-17A gene product(s) is detected and quantified to yield a test amount. The test amount is then compared with a normal amount or range. An amount significantly above the normal amount or range is a positive sign in the diagnosis of disorders related to IL-17F/IL-17A signaling. Particular methods of detection and quantification of IL-17F/IL-17A gene products are described below.

Normal amounts or baseline levels of IL-17F/IL-17A gene products may be determined for any particular sample type and population. Generally, baseline (normal) levels of IL-17F/IL-17A gene product(s) are determined by measuring respective amounts of IL-17F/IL-17A gene product(s) in a biological sample type from normal (e.g., healthy) subjects. Alternatively, normal values of IL-17F/IL-17A gene product(s) may be determined by measuring the amount in healthy cells or tissues taken from the same subject from which the diseased (or possibly diseased) test cells or tissues were taken. The amount of IL-17F/IL-17A gene product(s) (either the normal amount or the test amount) may be determined or expressed on a per cell, per total protein, or per volume basis. To determine the cell amount of a sample, one can measure the level of a constitutively expressed gene product or other gene product expressed at known levels in cells of the type from which the biological sample was taken.

It will be appreciated that the assay methods of the present invention do not necessarily require measurement of absolute values of IL-17F/IL-17A gene product(s) because relative values are sufficient for many applications of these methods. It will also be appreciated that in addition to the quantity or abundance of IL-17F/IL-17A gene product(s), variant or abnormal IL-17F/IL-17A gene products or their expression patterns (e.g., mutated transcripts, truncated polypeptides) may be identified by comparison to normal gene product(s) and expression patterns.

Whether the expression of a particular gene in two samples is significantly similar or significantly different, e.g., significantly above or significantly below a given level, depends on the gene itself and, inter alia, its variability in expression between different individuals or different samples. It is within the skill in the art to determine whether expression levels are significantly similar or different. Factors such as genetic variation, e.g., in IL-17F/IL-17A expression levels, between individuals, species, organs, tissues, or cells may be taken into consideration (when and where necessary) when determining whether the level of expression, e.g., of IL-17F/IL-17A, between two samples is significantly similar or significantly different, e.g., significantly above a given level. As a result of the natural heterogeneity in gene expression between individuals, species, organs, tissues, or cells, phrases such as “significantly similar” or “significantly above” or the like cannot be defined as a precise percentage or value, but rather can be ascertained by one skilled in the art upon practicing the invention.

The diagnostic, prognostic, and monitoring assays of the present invention involve detecting and quantifying IL-17F/IL-17A gene product(s) in biological samples. IL-17F/IL-17A gene products include mRNAs, cDNAs (e.g., IL-17A and IL-17F mRNA and/or cDNA) and/or polypeptides (e.g., IL-17F/IL-17A, IL-17F, IL-17A polypeptides), and both can be measured using methods well known to those skilled in the art.

For example, mRNA can be directly detected and quantified using hybridization-based assays, such as Northern hybridization, in situ hybridization, dot and slot blots, and oligonucleotide arrays. Hybridization-based assays refer to assays in which a probe nucleic acid is hybridized to a target nucleic acid. In some formats, the target, the probe, or both are immobilized. The immobilized nucleic acid may be DNA, RNA, or another oligonucleotide or polynucleotide, and may comprise naturally or nonnaturally occurring nucleotides, nucleotide analogs, or backbones. Methods of selecting nucleic acid probe sequences for use in the present invention are based on the nucleic acid sequence of IL-17F and/or IL-17A, and are well known in the art.

Alternatively, mRNA can be amplified before detection and quantitation. Such amplification-based assays are well known in the art and include polymerase chain reaction (PCR), reverse-transcription-PCR (RT-PCR), PCR-enzyme-linked immunosorbent assay (PCR-ELISA), and ligase chain reaction (LCR). Primers and probes for producing and detecting amplified IL-17A and/or IL-17F gene products (e.g., mRNA or cDNA) may be readily designed and produced without undue experimentation by those of skill in the art based on the nucleic acid sequences of IL-17A and IL-17F, respectively. As a nonlimiting example, amplified IL-17A and/or IL-17F gene products may be directly analyzed, for example, by gel electrophoresis; by hybridization to a probe nucleic acid; by sequencing; by detection of a fluorescent, phosphorescent, or radioactive signal; or by any of a variety of well-known methods. In addition, methods are known to those of skill in the art for increasing the signal produced by amplification of target nucleic acid sequences. One of skill in the art will recognize that whichever amplification method is used, a variety of quantitative methods known in the art (e.g., quantitative PCR) may be used if quantitation of gene products is desired.

IL-17F/IL-17A polypeptides (or fragments thereof) may be detected using various well-known immunological assays employing anti-IL-17A, anti-IL-17F, and/or anti-IL-17F/IL-17A antibodies, that may be generated as described herein. Immunological assays refer to assays that utilize an antibody (e.g., polyclonal, monoclonal, chimeric, humanized, scFv, and/or fragments thereof) that specifically binds to, e.g., an IL-17F/IL-17A polypeptide (or a fragment thereof). Such well-known immunological assays suitable for the practice of the present invention include ELISA, radioimmunoassay (RIA), immunoprecipitation, immunofluorescence, fluorescence-activated cell sorting (FACS), and Western blotting. An IL-17F/IL-17A polypeptide may also be detected using a combination of anti-IL-17A and anti-IL-17F antibodies, utilizing, e.g., a sandwich ELISA. Additionally, an IL-17F/IL-17A polypeptide may be detected using a labeled IL-17R and/or IL-17RC polypeptide(s). Conversely, IL-17R or IL-17RC may be detected using a labeled IL-17F/IL-17A polypeptide.

One of skill in the art will understand that the aforementioned methods may be applied to disorders related to IL-17F/IL-17A signaling. Uses of Molecules Related to IL-17F/IL-17A in Therapy

U.S. patent application Ser. No. 11/353,161, incorporated herein in its entirety by reference, demonstrates that both hIL-17F and hIL-17A induce similar responses through binding to the hIL-17R and/or hIL-17RC receptors. The present inventors demonstrate for the first time that hIL-17F/IL-17A heterodimer also binds to the same hIL-7R and hIL-17RC receptors, and elicits responses similar to the hIL-17A and hIL-17F homodimers. Further, the inventors provide a novel mouse IL-17F/IL-17A heterodimer, demonstrate that the heterodimer is biologically active in vivo, and that blockade of the heterodimer can be used in vivo to treat and/or prevent IL-17F/IL-17A-associated disorders, e.g., airway inflammation. Thus, one skilled in the art would recognize that the IL-17F/IL-17A-associated disorders may also include IL-17A- and IL-17F-associated disorders, and thus, may be treated with IL-17F/IL-17A signaling antagonists.

Molecules that modulate IL-17F/IL-17A signaling, disclosed herein, including modulators identified using the methods described above, may be used in vitro, ex vivo, or incorporated into pharmaceutical compositions and administered to subjects or individuals in vivo to treat, for example, disorders related to IL-17F/IL-17A signaling, by administration of an IL-17F/IL-17A signaling antagonist (e.g., IL-17A and/or IL-17F inhibitory polynucleotides; soluble IL-17R and/or IL-17RC polypeptides (including fragments and/or fusion proteins thereof); inhibitory anti-IL-17F, anti-IL17A, anti-IL-17F/IL-17A, anti-IL-17R, or anti-IL-17RC antibodies; antagonistic small molecules; etc.). Several pharmacogenomic approaches to be considered in determining whether to administer molecules that modulate IL-17F/IL-17A signaling are well known to one of skill in the art and include genome-wide association, candidate gene approach, and gene expression profiling. A pharmaceutical composition of the invention is formulated to be compatible with its intended route of administration (e.g., oral compositions generally include an inert diluent or an edible carrier). Other nonlimiting examples of routes of administration include parenteral (e.g., intravenous), intradermal, subcutaneous, oral (e.g., inhalation), transdermal (topical), transmucosal, and rectal administration. The pharmaceutical compositions compatible with each intended route are well known in the art.

IL-17F/IL-17A signaling agonists or IL-17F/IL-17A signaling antagonists may be used as pharmaceutical compositions when combined with a pharmaceutically acceptable carrier. Such a composition may contain, in addition to a molecule that modulates IL-17F/IL-17A (e.g., IL-17F/IL-17A signaling agonists or IL-17F/IL-17A signaling antagonists) and carrier, various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration.

The pharmaceutical composition of the invention may also contain cytokines, lymphokines, or other hematopoietic factors such as M-CSF, GM-CSF, IL-1, IL-2, IL-3, IL-4, IL-5, IL-6, IL-7, IL-8, IL-9, IL-10, IL-11, IL-12, IL-14, IL-15, G-CSF, stem cell factor, and erythropoietin. The pharmaceutical composition may also include anti-cytokine antibodies as described in more detail below. The pharmaceutical composition may contain thrombolytic or antithrombotic factors such as plasminogen activator and Factor VIII. The pharmaceutical composition may further contain other anti-inflammatory agents as described in more detail below. Such additional factors and/or agents may be included in the pharmaceutical composition to produce a synergistic effect with IL-17F/IL-17A signaling agonists or IL-17F/IL-17A signaling antagonists, or to minimize side effects caused by the IL-17F/IL-17A signaling agonists or IL-17F/IL-17A signaling antagonists. Conversely IL-17F/IL-17A signaling agonists or IL-17F/IL-17A signaling antagonists may be included in formulations of the particular cytokine, lymphokine, other hematopoietic factor, thrombolytic or antithrombotic factor, or anti-inflammatory agent to minimize side effects of the cytokine, lymphokine, other hematopoietic factor, thrombolytic or antithrombotic factor, or anti-inflammatory agent.

The pharmaceutical composition of the invention may be in the form of a liposome in which IL-17F/IL-17A signaling agonist(s) or IL-17F/IL-17A signaling antagonist(s) are combined with, in addition to other pharmaceutically acceptable carriers, amphipathic agents such as lipids that exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, etc.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of an IL-17F/IL-17A signaling modulator (e.g., IL-17F/IL-17A signaling agonist or IL-17F/IL-17A signaling antagonist) is administered to a subject, e.g., a mammal (e.g., a human). An IL-17F/IL-17A signaling modulator may be administered in accordance with the methods of the invention either alone or in combination with other therapies, such as treatments employing cytokines, lymphokines or other hematopoietic factors, or anti-inflammatory agents. When coadministered with one or more agents, IL-17F/IL-17A agonists or antagonists may be administered either simultaneously with the second agent, or sequentially. If administered sequentially, the attending physician will decide on the appropriate sequence of administering, e.g., a specific anti-IL-17F/IL-17A antagonistic antibody in combination with other agents.

When a therapeutically effective amount of an IL-17F/IL-17A modulator is administered orally, the binding agent will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% binding agent, and preferably from about 25 to 90% binding agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition contains from about 0.5 to 90% by weight of the binding agent, and preferably from about 1 to 50% by weight of the binding agent.

When a therapeutically effective amount of an IL-17F/IL-17A modulator is administered by intravenous, cutaneous or subcutaneous injection, the IL-17F/IL-17A modulator will be in the form of a pyrogen-free, parenterally acceptable aqueous solution. The preparation of such parenterally acceptable protein solutions, having due regard to pH, isotonicity, stability, and the like, is within the skill of those in the art. A preferred pharmaceutical composition for intravenous, cutaneous, or subcutaneous injection should contain, in addition to the IL-17F/IL-17A modulator, an isotonic vehicle such as sodium chloride injection, Ringer's injection, dextrose injection, dextrose and sodium chloride injection, lactated Ringer's injection, or other vehicle as known in the art. The pharmaceutical composition of the present invention may also contain stabilizers, preservatives, buffers, antioxidants, or other additive known to those of skill in the art.

The amount of an IL-17F/IL-17A modulator in the pharmaceutical composition of the present invention will depend upon the nature and severity of the condition being treated, and on the nature of prior treatments that the patient has undergone. Ultimately, the attending physician will decide the amount of IL-17F/IL-17A modulator with which to treat each individual patient. Initially, the attending physician will administer low doses of IL-17F/IL-17A modulator and observe the patient's response. Larger doses of IL-17F/IL-17A modulator may be administered until the optimal therapeutic effect is obtained for the patient, and at that point the dosage is not generally increased further. It is contemplated that the various pharmaceutical compositions used to practice the method of the present invention should contain about 0.1 μg to about 100 mg of IL-17F/IL-17A modulator, e.g., specific antagonistic anti-IL-17F/IL-17A antibody, per kg body weight.

The duration of intravenous (i.v.) therapy using a pharmaceutical composition of the present invention will vary, depending on the severity of the disease being treated and the condition and potential idiosyncratic response of each individual patient. It is contemplated that the duration of each application of the IL-17F/IL-17A modulator may be in the range of more than one hour of administration, e.g., about 12 to about 24 hours of continuous i.v. administration. Also contemplated is subcutaneous (s.c.) therapy using a pharmaceutical composition of the present invention. These therapies can be administered daily, weekly, or, more preferably, biweekly, or monthly. It is also contemplated that where the IL-17F/IL-17A modulator is a small molecule (e.g., for oral delivery), the therapies may be administered daily, twice a day, three times a day, etc. Ultimately the attending physician will decide on the appropriate duration of i.v. or s.c. therapy, or therapy with a small molecule, and the timing of administration of the therapy, using the pharmaceutical composition of the present invention.

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

### Uses of IL-17F/IL-17a Signaling Antagonists to Treat Immune Disorders

IL-17F/IL-17A signaling antagonists may also be administered to subjects for whom suppression of IL-17F/IL-17A signaling is desired. These conditions include, but are not limited to, inflammatory disorders, e.g., autoimmune diseases (e.g., arthritis (including rheumatoid arthritis), psoriasis, systemic lupus erythematosus, multiple sclerosis), respiratory diseases (e.g., airway inflammation, COPD, cystic fibrosis, asthma, allergy), transplant rejection (including solid organ transplant rejection), and inflammatory bowel diseases (e.g., ulcerative colitis, Crohn's disease).

These methods are based in part on the finding that treating cells with hIL-17F, hIL-17A, and/or hIL-17F/IL-17A antagonists related to the invention, (e.g., hIL-17R.Fc, hIL-17RC.Fc, anti-hIL-17R antibody, anti-hIL-7RC antibody,) inhibits hIL-17A-, hIL-17F-, and/or hIL-17F/IL-17A-induced cytokine release, e.g., GRO-α cytokine release, e.g., from human foreskin fibroblast cells (Examples 1.2.4-1.2.5). In addition, these methods are based in part on the finding that treating cells with inhibitory polynucleotides related to the present invention (e.g., IL-17R siRNA and IL-17RC siRNA), inhibits hIL-17A-, hIL-17F-, and hIL-17F/IL-17A-induced cytokine release (Example 1.2.6). Further, the inventors demonstrate that IL-17F/IL-17A plays a role in airway inflammation in vivo, and that blockade of the cytokine prevents and/or reduces such airway inflammation (Examples 2.2.3-2.2.5). Accordingly, IL-17F/IL-17A antagonists (e.g., IL-17F/IL-17A signaling antagonists), i.e., molecules that inhibit IL-17F/IL-17A biological activity, may be used to decrease inflammation in vivo, e.g., for treating or preventing IL-17F/IL-17A-associated disorders, e.g., disorders related to IL-17F/IL-17A signaling.

The methods of using IL-17F/IL-17A signaling antagonists may also be used inhibit IL-17F/IL-17A biological activity in immune disorders and thus, can be used to treat or prevent a variety of immune disorders. Nonlimiting examples of the disorders that can be treated or prevented include, but are not limited to, transplant rejection, autoimmune diseases (including, e.g., diabetes mellitus, arthritis (including rheumatoid arthritis, juvenile rheumatoid arthritis, osteoarthritis, psoriatic arthritis, reactive arthritis), multiple sclerosis, encephalomyelitis, myasthenia gravis, systemic lupus erythematosus (SLE), autoimmune thyroiditis, dermatitis (including atopic dermatitis and eczematous dermatitis), Reiter's syndrome, psoriasis, Sjögren's syndrome, Crohn's disease, aphthous ulcer, iritis, conjunctivitis, keratoconjunctivitis, ulcerative colitis, spondyloarthropathy, ankylosing spondylitis, intrinsic asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, pulmonary exacerbation (e.g., due to bacterial infection), and allergy, such as atopic allergy. Preferred disorders that can be treated using methods which comprise the administration of IL-17F/IL-17A signaling antagonists (e.g., antagonistic antibodies to IL-17A, IL-17F and/or IL-17F/IL-17A and fragments thereof; soluble receptors; small molecules; inhibitory polynucleotides; etc.) that interfere with IL-17A, IL-17F, and/or IL-17F/IL-17A heterodimer signaling, include, but are not limited to, inflammatory disorders, e.g., autoimmune diseases (e.g., arthritis (including rheumatoid arthritis), psoriasis, systemic lupus erythematosus, multiple sclerosis), respiratory diseases (e.g., airway inflammation, COPD, cystic fibrosis, asthma, allergy), transplant rejection (including solid organ transplant rejection), and inflammatory bowel diseases (e.g., ulcerative colitis, Crohn's disease).

Using IL-17F/IL-17A signaling antagonists (e.g., IL-17A, IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17A, anti-IL-17F/IL-17A, anti-IL-17R, or IL-17RC antibodies; and/or antagonistic small molecules, etc.), it is possible to modulate (e.g., downregulate) immune responses in a number of ways. Downregulation may be in the form of inhibiting or blocking an inflammatory response already in progress, or may involve preventing the induction of an inflammatory response.

In one embodiment, IL-17F/IL-17A signaling antagonists, including pharmaceutical compositions thereof, are administered in combination therapy, i.e., combined with other agents, e.g., therapeutic agents, that are useful for treating pathological conditions or disorders, such as immune disorders and inflammatory diseases. The term “in combination” in this context means that the agents are given substantially contemporaneously, either simultaneously or sequentially. If given sequentially, at the onset of administration of, e.g., the second compound, the first of the two compounds is preferably still detectable at effective concentrations at the site of treatment.

For example, the combination therapy can include one or more IL-17F/IL-17A signaling antagonists (e.g., IL-17A, IL-17F, IL-17R, and/or IL-17RC inhibitory polynucleotides; soluble IL-17R and/or IL-17RC polypeptides (including fragments and/or fusion proteins thereof); inhibitory anti-IL-17F, anti-IL-17A, anti-IL17F/IL-17A, anti-IL-17R, or IL-17RC antibodies; antagonistic small molecules; etc.) coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail herein. Furthermore, one or more IL-17F/IL-17A signaling antagonists described herein may be used in combination with two or more of the therapeutic agents described herein. Such combination therapies may advantageously utilize lower dosages of the administered therapeutic agents, thus avoiding possible toxicities or complications associated with the various monotherapies. Moreover, the therapeutic agents disclosed herein act on pathways that differ from the IL-17F/IL-17A receptor signaling pathway, and thus, are expected to enhance and/or synergize with the effects of the IL-17F/IL-17A signaling antagonists.

Preferred therapeutic agents used in combination with an IL-17F/IL-17A signaling antagonist are those agents that interfere at different stages in an inflammatory response. In one embodiment, one or more IL-17F/IL-17A signaling antagonists described herein may be coformulated with, and/or coadministered with, one or more additional agents such as other cytokine or growth factor antagonists (e.g., soluble receptors, peptide inhibitors, small molecules, ligand fusions); or antibodies or antigen binding fragments thereof that bind to other targets (e.g., antibodies that bind to other cytokines or growth factors, their receptors, or other cell surface molecules); and anti-inflammatory cytokines or agonists thereof. Examples of the agents that can be used in combination with the IL-17F/IL-17A signaling antagonists described herein, include, but are not limited to, antagonists of one or more interleukins (ILs) or their receptors, e.g., antagonists of IL-1, IL-2, IL-6, IL-7, IL-8, IL-12, IL-13, IL-15, IL-16, IL-18, IL-21 and IL-22; antagonists of cytokines or growth factors or their receptors, such as tumor necrosis factor (TNF), LT, EMAP-II, GM-CSF, FGF and PDGF. IL-17F/IL-17A signaling antagonists can also be combined with inhibitors of, e.g., antibodies to, cell surface molecules such as CD2, CD3, CD4, CD8, CD20 (e.g., the CD20 inhibitor rituximab (RITUXAN®)), CD25, CD28, CD30, CD40, CD45, CD69, CD80 (B7.1), CD86 (B7.2), CD90, or their ligands, including CD154 (gp39 or CD40L), or LFA-1/ICAM-1 and VLA-4/VCAM-1 (Yusuf-Makagiansar et al. (2002) Med. Res. Rev. 22:146-67). Preferred antagonists that can be used in combination with IL-17F/IL-17A signaling antagonists described herein include antagonists of IL-1, IL-12, TNFα, IL-15, IL-18, and IL-22.

Examples of those agents include IL-12 antagonists, such as chimeric, humanized, human or in vitro-generated antibodies (or antigen binding fragments thereof) that bind to IL-12 (preferably human IL-12), e.g., the antibody disclosed in WO 00/56772; IL-12 receptor inhibitors, e.g., antibodies to human IL-12 receptor; and soluble fragments of the IL-12 receptor, e.g., human IL-12 receptor. Examples of IL-15 antagonists include antibodies (or antigen binding fragments thereof) against IL-15 or its receptor, e.g., chimeric, humanized, human or in vitro-generated antibodies to human IL-15 or its receptor, soluble fragments of the IL-15 receptor, and IL-15-binding proteins. Examples of IL-18 antagonists include antibodies, e.g., chimeric, humanized, human or in vitro-generated antibodies (or antigen binding fragments thereof), to human IL-18, soluble fragments of the IL-18 receptor, and IL-18 binding proteins (IL-18BP). Examples of IL-1 antagonists include Interleukin-1-converting enzyme (ICE) inhibitors, such as Vx740, IL-1 antagonists, e.g., IL-IRA (anikinra, KINERET™, Amgen), sIL1RII (Immunex), and anti-IL-1 receptor antibodies (or antigen binding fragments thereof).

Examples of TNF antagonists include chimeric, humanized, human or in vitro-generated antibodies (or antigen binding fragments thereof) to TNF (e.g., human TNFα), such as (HUMIRA™, D2E7, human TNFα antibody), CDP-571/CDP-870/BAY-10-3356 (humanized anti-TNFα antibody; Celltech/Pharmacia), cA2 (chimeric anti-TNFα antibody; REMICADE®, Centocor); anti-TNF antibody fragments (e.g., CPD870); soluble fragments of the TNF receptors, e.g., p55 or p75 human TNF receptors or derivatives thereof, e.g., 75 kd TNFR-IgG (75 kD TNF receptor-IgG fusion protein, ENBREL™; Immunex), p55 kd TNFR-IgG (55 kD TNF receptor-IgG fusion protein (LENERCEPT®)); enzyme antagonists, e.g., TNFα converting enzyme (TACE) inhibitors (e.g., an alpha-sulfonyl hydroxamic acid derivative, and N-hydroxyformamide TACE inhibitor GW 3333, -005, or -022); and TNF-bp/s-TNFR (soluble TNF binding protein). Preferred TNF antagonists are soluble fragments of the TNF receptors, e.g., p55 or p75 human TNF receptors or derivatives thereof, e.g., 75 kd TNFR-IgG, and TNFα converting enzyme (TACE) inhibitors.

In other embodiments, IL-17F/IL-17A signaling antagonists described herein may be administered in combination with one or more of the following: IL-13 antagonists, e.g., soluble IL-13 receptors (sIL-13) and/or antibodies against IL-13; IL-2 antagonists, e.g., DAB 486-IL-2 and/or DAB 389-IL-2 (IL-2 fusion proteins, Seragen), and/or antibodies to IL-2R, e.g., anti-Tac (humanized anti-IL-2R, Protein Design Labs). Yet another combination includes IL-17F/IL-17A signaling antagonists in combination with nondepleting anti-CD4 inhibitors (IDEC-CE9.1/SB 210396; nondepleting primatized anti-CD4 antibody; IDEC/SmithKline). Yet other preferred combinations include antagonists of the costimulatory pathway CD80 (B7.1) or CD86 (B7.2), including antibodies, soluble receptors or antagonistic ligands; as well as p-selectin glycoprotein ligand (PSGL), anti-inflammatory cytokines, e.g., IL-4 (DNAX/Schering); IL-10 (SCH 52000; recombinant IL-10 DNAX/Schering); IL-13 and TGF-β, and agonists thereof (e.g., agonist antibodies).

In other embodiments, one or more IL-17F/IL-17A signaling antagonists can be coformulated with, and/or coadministered with, one or more anti-inflammatory drugs, immunosuppressants, or metabolic or enzymatic inhibitors. Nonlimiting examples of the drugs or inhibitors that can be used in combination with the IL-17F/IL-17A signaling antagonists described herein, include, but are not limited to, one or more of: nonsteroidal anti-inflammatory drug(s) (NSAIDs), e.g., ibuprofen, tenidap, naproxen, meloxicarn, piroxicam, diclofenac, and indomethacin; sulfasalazine; corticosteroids such as prednisolone; cytokine suppressive anti-inflammatory drug(s) (CSAIDs); inhibitors of nucleotide biosynthesis, e.g., inhibitors of purine biosynthesis, folate antagonists (e.g., methotrexate (N-[4-[[(2,4-diamino-6-pteridinyl)methyl]methylamino]benzoyl]-L-glutamic acid); and inhibitors of pyrimidine biosynthesis, e.g., dihydroorotate dehydrogenase (DHODH) inhibitors. Preferred therapeutic agents for use in combination with IL-17F/IL-17A signaling antagonists include NSAIDs, CSAIDs, (DHODH) inhibitors (e.g., leflunomide), and folate antagonists (e.g., methotrexate).

Examples of additional inhibitors include one or more of: corticosteroids (oral, inhaled and local injection); immunosuppresants, e.g., cyclosporin, tacrolimus (FK-506); and mTOR inhibitors, e.g., sirolimus (rapamycin—RAPAMUNE™ or rapamycin derivatives, e.g., soluble rapamycin derivatives (e.g., ester rapamycin derivatives, e.g., CCI-779); agents which interfere with signaling by proinflammatory cytokines such as TNFα or IL-1 (e.g. IRAK, NIK, IKK, p38 or MAP kinase inhibitors); COX2 inhibitors, e.g., celecoxib, rofecoxib, and variants thereof; phosphodiesterase inhibitors, e.g., R973401 (phosphodiesterase Type IV inhibitor); phospholipase inhibitors, e.g., inhibitors of cytosolic phospholipase 2 (cPLA2) (e.g., trifluoromethyl ketone analogs); inhibitors of vascular endothelial cell growth factor or growth factor receptor, e.g., VEGF inhibitor and/or VEGF-R inhibitor; and inhibitors of angiogenesis. Preferred therapeutic agents for use in combination with IL-17F/IL-17A signaling antagonists are immunosuppressants, e.g., cyclosporin, tacrolimus (FK-506); mTOR inhibitors, e.g., sirolimus (rapamycin) or rapamycin derivatives, e.g., soluble rapamycin derivatives (e.g., ester rapamycin derivatives, e.g., CCI-779); COX2 inhibitors, e.g., celecoxib and variants thereof; and phospholipase inhibitors, e.g., inhibitors of cytosolic phospholipase 2 (cPLA2), e.g., trifluoromethyl ketone analogs.

Additional examples of therapeutic agents that can be combined with an IL-17F/IL-17A signaling antagonist include one or more of: 6-mercaptopurines (6-MP); azathioprine sulphasalazine; mesalazine; olsalazine; chloroquine/hydroxychloroquine (PLAQUENIL®); penicillamine; aurothiomalate (intramuscular and oral); azathioprine; colchicine; beta-2 adrenoreceptor agonists (salbutamol, terbutaline, salmeterol); xanthines (theophylline, aminophylline); cromoglycate; nedocromil; ketotifen; ipratropium and oxitropium; mycophenolate mofetil; adenosine agonists; antithrombotic agents; complement inhibitors; and adrenergic agents.

The use of the IL-17F/IL-17A signaling antagonists disclosed herein in combination with other therapeutic agents to treat or prevent specific disorders related to IL-17F/IL-17A signaling is discussed in further detail below.

Nonlimiting examples of agents for treating or preventing arthritic disorders (e.g., rheumatoid arthritis, inflammatory arthritis, rheumatoid arthritis, juvenile rheumatoid arthritis, osteoarthritis and psoriatic arthritis), with which IL-17F/IL-17A signaling antagonists may be combined include one or more of the following: IL-12 antagonists as described herein; NSAIDs; CSAIDs; TNFs, e.g., TNFα antagonists as described herein; nondepleting anti-CD4 antibodies as described herein; IL-2 antagonists as described herein; anti-inflammatory cytokines, e.g., IL-4, IL-10, IL-13 and TGFα, or agonists thereof; IL-1 or IL-1 receptor antagonists as described herein; phosphodiesterase inhibitors as described herein; Cox-2 inhibitors as described herein; iloprost: methotrexate; thalidomide and thalidomide-related drugs (e.g., Celgen); leflunomide; inhibitor of plasminogen activation, e.g., tranexamic acid; cytokine inhibitor, e.g., T-614; prostaglandin E1; azathioprine; an inhibitor of interleukin-1 converting enzyme (ICE); zap-70 and/or Ick inhibitor (inhibitor of the tyrosine kinase zap-70 or Ick); an inhibitor of vascular endothelial cell growth factor or vascular endothelial cell growth factor receptor as described herein; an inhibitor of angiogenesis as described herein; corticosteroid anti-inflammatory drugs (e.g., SB203580); TNF-convertase inhibitors; IL-11; IL-13; IL-17 inhibitors; gold; penicillamine; chloroquine; hydroxychloroquine; chlorambucil; cyclophosphamide; cyclosporine; total lymphoid irradiation; antithymocyte globulin; CD5-toxins; orally administered peptides and collagen; lobenzarit disodium; cytokine regulating agents (CRAs) HP228 and HP466 (Houghten Pharmaceuticals, Inc.); ICAM-1 antisense phosphorothioate oligodeoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline (MINOCIN®); anti-IL2R antibodies; marine and botanical lipids (fish and plant seed fatty acids); auranofin; phenylbutazone; meclofenamic acid; flufenamic acid; intravenous immune globulin; zileuton; mycophenolic acid (RS-61443); tacrolimus (FK-506); sirolimus (rapamycin); amiprilose (therafectin); cladribine (2-chlorodeoxyadenosine); and azaribine. Preferred combinations include one or more IL-17F/IL-17A signaling antagonists in combination with methotrexate or leflunomide, and in moderate or severe rheumatoid arthritis cases, cyclosporine.

Preferred examples of inhibitors to use in combination with IL-17F/IL-17A signaling antagonists to treat arthritic disorders include TNF antagonists (e.g., chimeric, humanized, human or in vitro-generated antibodies, or antigen binding fragments thereof, that bind to TNF; soluble fragments of a TNF receptor, e.g., p55 or p75 human TNF receptor or derivatives thereof, e.g., 75 kd TNFR-IgG (75 kD TNF receptor-IgG fusion protein, ENBREL™), p55 kD TNF receptor-IgG fusion protein; TNF enzyme antagonists, e.g., TNFα converting enzyme (TACE) inhibitors); antagonists of IL-12, IL-15, IL-18, IL-22; T cell and B cell-depleting agents (e.g., anti-CD4 or anti-CD22 antibodies); small molecule inhibitors, e.g., methotrexate and leflunomide; sirolimus (rapamycin) and analogs thereof, e.g., CCI-779; COX-2 and cPLA2 inhibitors; NSAIDs; p38 inhibitors, TPL-2, Mk-2 and NFκB inhibitors; RAGE or soluble RAGE; P-selectin or PSGL-1 inhibitors (e.g., small molecule inhibitors, antibodies thereto, e.g., antibodies to P-selectin); estrogen receptor beta (ERB) agonists or ERB-NFκB antagonists. Most preferred additional therapeutic agents that can be coadministered and/or coformulated with one or more IL-17F/IL-17A signaling antagonists include one or more of: a soluble fragment of a TNF receptor, e.g., p55 or p75 human TNF receptor or derivatives thereof, e.g., 75 kd TNFR-IgG (75 kD TNF receptor-IgG fusion protein, ENBREL™); methotrexate, leflunomide, or a sirolimus (rapamycin) or an analog thereof, e.g., CCI-779.

Nonlimiting examples of agents for treating or preventing multiple sclerosis with which IL-17F/IL-17A signaling antagonists can be combined include the following: interferons, e.g., interferon-alphala (e.g., AVONEX™; Biogen) and interferon-1b (BETASERON Chiron/Berlex); Copolymer 1 (Cop-1; COPAXONE™ Teva Pharmaceutical Industries, Inc.); hyperbaric oxygen; intravenous immunoglobulin; cladribine; TNF antagonists as described herein; corticosteroids; prednisolone; methylprednisolone; azathioprine; cyclophosphamide; cyclosporine; cyclosporine A, methotrexate; 4-aminopyridine; and tizanidine. Additional antagonists that can be used in combination with antagonists of IL-17F/IL-17A signaling include antibodies to or antagonists of other human cytokines or growth factors, for example, TNF, LT, IL-1, IL-2, IL-6, IL-7, IL-8, IL-12 IL-15, IL-16, IL-18, EMAP-11, GM-CSF, FGF, and PDGF. IL-17F/IL-17A signaling antagonists as described herein can be combined with antibodies to cell surface molecules such as CD2, CD3, CD4, CD8, CD25, CD28, CD30, CD40, CD45, CD69, CD80, CD86, CD90 or their ligands. The IL-17F/IL-17A signaling antagonists may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signaling by proinflammatory cytokines as described herein, IL-1b converting enzyme inhibitors (e.g., Vx740), anti-P7s, PSGL, TACE inhibitors, T-cell signaling inhibitors such as kinase inhibitors, metalloproteinase inhibitors, sulfasalazine, azathloprine, 6-mercaptopurines, angiotensin converting enzyme inhibitors, soluble cytokine receptors and derivatives thereof, as described herein, and anti-inflammatory cytokines (e.g. IL-4, IL-10, IL-13 and TGF).

Preferred examples of therapeutic agents for multiple sclerosis with which the IL-17F/IL-17A signaling antagonists can be combined include interferon-β, for example, IFNβ-1a and IFNβ-Ib; copaxone, corticosteroids, IL-1 inhibitors, TNF inhibitors, antibodies to CD40 ligand and CD80, IL-12 antagonists.

Nonlimiting examples of agents for treating or preventing inflammatory bowel disease (e.g., Crohn's disease, ulcerative colitis) with which an IL-17F/IL-17A signaling antagonist can be combined include the following: budenoside; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 antibodies; anti-IL-6 antibodies; anti-IL-22 antibodies; growth factors; elastase inhibitors; pyridinyl-imidazole compounds; TNF antagonists as described herein; IL-4, IL-10, IL-13 and/or TGFβ cytokines or agonists thereof (e.g., agonist antibodies); IL-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of platelet activating factor (PAF); ciprofloxacin; and lignocaine.

Nonlimiting examples of agents for treating or preventing inflammatory diseases and disorders of the skin (including but not limited to psoriasis) with which an IL-17F/IL-17A signaling antagonist can be combined include the following: antagonists of IL-12, IL-15, IL-18, and IL-22.

In one embodiment, an IL-17F/IL-17A signaling antagonist can be used in combination with one or more antibodies directed at other targets involved in regulating immune responses, e.g., transplant rejection. Nonlimiting examples of agents for treating or preventing immune responses with which an IL-17F/IL-17A signaling antagonist of the invention can be combined include the following: antibodies against other cell surface molecules, including but not limited to CD25 (interleukin-2 receptor-a), CD11a (LFA-1), CD54 (ICAM-1), CD4, CD45, CD28/CTLA4 (CD80 (B7.1), e.g., CTLA4 Ig—abatacept (ORENCIA®)), ICOSL, ICOS and/or CD86 (B7.2). In yet another embodiment, an IL-17F/IL-17A signaling antagonist is used in combination with one or more general immunosuppressive agents, such as cyclosporin A or FK506.

In another embodiment of the invention, an IL-17F/IL-17A signaling antagonist is used in combination with methods of downregulating antigen presenting cell fusion and/or therapy for managing immunosuppression. Methods of: 1) downregulating antigen presenting cell function; and 2) combination therapy for managing immunosuppression are well known in the art (see, e.g., Xiao et al. (2003) BioDrugs 17:103-11; Kuwana (2002) Hum. Immunol. 63:1156-63; Lu et al. (2002) Transplantation 73:S19-22; Rifle et al. (2002) Transplantation 73:S1-S2; Mancini et al. (2004) Crit. Care. Nurs. Q. 27:61-64).

In other embodiments, IL-17F/IL-17A signaling antagonists are used as vaccine adjuvants against autoimmune disorders, inflammatory diseases, etc. The combination of adjuvants for treatment of these types of disorders are suitable for use in combination with a wide variety of antigens from targeted self-antigens, i.e., autoantigens, involved in autoimmunity, e.g., myelin basic protein; inflammatory self-antigens, e.g., amyloid peptide protein, or transplant antigens, e.g., alloantigens. The antigen may comprise peptides or polypeptides derived from proteins, as well as fragments of any of the following: saccharides, proteins, polynucleotides or oligonucleotides, autoantigens, amyloid peptide protein, transplant antigens, allergens, or other macromolecular components. In some instances, more than one antigen is included in the antigenic composition.

For example, desirable vaccines for moderating responses to allergens in a vertebrate host, which contain the adjuvant combinations of this invention, include those containing an allergen or fragment thereof. Examples of such allergens are described in U.S. Pat. No. 5,830,877 and published International Patent Application No. WO 99/51259, which are hereby incorporated by reference in their entireties, and include pollen, insect venoms, animal dander, fungal spores and drugs (such as penicillin). The vaccines interfere with the production of IgE antibodies, a known cause of allergic reactions. In another example, desirable vaccines for preventing or treating disease characterized by amyloid deposition in a vertebrate host, which contain the adjuvant combinations of this invention, include those containing portions of amyloid peptide protein (APP). This disease is referred to variously as Alzheimer's disease, amyloidosis or amyloidogenic disease. Thus, the vaccines of this invention include, for example, the adjuvant combinations of this invention plus Aβ peptide, as well as fragments of Aβ peptide and antibodies to Aβ peptide or fragments thereof.

Another aspect of the present invention accordingly relates to kits for carrying out the administration of the IL-17F/IL-17A signaling antagonists with other therapeutic compounds. In one embodiment, the kit comprises one or more binding agents formulated in a pharmaceutical carrier, and at least one agent, e.g., therapeutic agent, formulated as appropriate, in one or more separate pharmaceutical preparations.

The entire contents of all references, patents, and patent applications cited throughout this application are hereby incorporated by reference herein.

### EXAMPLES

The following Examples provide illustrative embodiments of the invention and do not in any way limit the invention. One of ordinary skill in the art will recognize that numerous other embodiments are encompassed within the scope of the invention.

The Examples do not include detailed descriptions of conventional methods, such methods employed in the construction of vectors, the insertion of genes encoding the polypeptides into such vectors and plasmids, the introduction of such vectors and plasmids into host cells, and the expression of polypeptides from such vectors and plasmids in host cells. Such methods are well known to those of ordinary skill in the art

### Example 1

### The Novel Heterodimeric Human Cytokine IL-17F/IL-17A Requires the Human Heteroreceptor Complex IL-17R and IL-17RC for its Functional Activity

### Example 1.1

### Materials and Methods

### Example 1.1.1

### Reagents

Human IL-17R.Fc and hIL-17RC.Fc were purchased from R&D Systems (Minneapolis, Minn.). Human IL-17F, hIL-17A and hIL-17F/IL-17A were purified according to the methods previously described (U.S. patent application Ser. No. 11/353,161; Wright et al. (2007) J. Biol. Chem. 282:13447-55, both incorporated by reference herein in their entireties). hIL-17F, hIL-17A and hIL-17F/IL-17A were biotinylated using FLUOREPORTER® Mini-biotin-XX Protein Labeling Kit according to the manufacture's protocol (Cat. # F-6347, Molecular Probes, Grand Island, N.Y.).

### Example 1.1.2

### Cloning of Human IL-17 Receptor Fusion Proteins

Full-length human IL-17R and hIL-17RC were PCR amplified from cDNA made from unstimulated MG63 cells. Sequencing confirmed a nucleic acid sequence of human IL-17RC matching NCBI Accession No. AY359098 (SEQ ID NO:26, which encodes a 705 amino acid protein set forth as SEQ ID NO:27) and a nucleic acid sequence of human IL-17R matching NCBI Accession No. BCO11624 (SEQ ID NO:28, which encodes an 866 amino acid protein set forth in SEQ ID NO:29). The full-length clones were each subcloned into a retroviral construct and were also used as templates for the generation of soluble fusion proteins. The extracellular portion of human IL-17R (residues 1-317 of SEQ ID NO:29) was fused in frame with a linker (GSGSGSG, SEQ ID NO:30) and the human IgG1 Fc (nucleic acid sequence set forth as SEQ ID NO:31, amino acid sequence set forth as SEQ ID NO:32). The extracellular portion of human IL-17RC (residues 1-452; of SEQ ID NO:27) was fused in frame with linker (AGSGSGSG, SEQ ID NO:33) and the human IgG1 Fc. These PCR-derived fusion receptors were separately subcloned into CMV promoter-driven mammalian expression constructs. All constructs were sequence verified.

### Example 1.1.3

### Expression of Human IL-17 Receptor Fusion Proteins

Proteins were expressed by transient transfection of HEK293 cells (TransIT-LT1, Mirus, Madison, Wis.). Twenty-four hours after transfection, media containing the DNA/liposome mixture was removed and replaced with serum-free media. The conditioned media was harvested 48 hours later and protein production was evaluated by Western analysis.

### Example 1.1.4

### Purification of Human IL-17 Receptor Fusion Proteins

Medium containing human IL-17R.Fc or hIL-17RC.Fc was flowed over a Protein A column (Amersham, Piscataway, N.J.). The column was washed with PBS and the fusion protein was eluted with 20 mM citric acid, 200 mM NaCl, pH 3. IL-17R.Fc aggregates were removed by passing the protein over a size exclusion column using a PBS pH 7.2 running buffer. The proteins were dialyzed against PBS pH 7.2 and were characterized by SDS-PAGE, Western analysis and analytical size-exclusion chromatography.

### Example 1.1.5

### ELISAs of Human IL-17A, IL-17F, or IL-17F/IL-17A Binding to Human IL-17R.Fc and Human IL-17RC.Fc

Binding of human IL-17F, hIL-17A or hIL-17F/IL-17A to human IL-17R.Fc (hIL-17R.Fc) and IL-17RC.Fc (hIL-17RC.Fc) was determined by indirect sandwich ELISA. ELISA plates (Costar, Cambridge Mass.) were coated overnight with 10 μg/ml goat anti-human IgG-Fc (Bethyl Laboratories, Montgomery, Tex.). Human IL-17R.Fc or hIL17-RC.Fc was then loaded at 6 ng/ml and 30 ng/ml, respectively, for 3 hours, followed by serial dilutions of biotinylated IL-17A, IL-17F or IL-17F/IL-17A for 2 hours. The plate was developed with Poly-HRP Streptavidin (Pierce Biotechnology, Rockford, Ill.) and TMB Substrate (KPL Labs, Gaithersburg, Md.).

### Example 1.1.6

### Cell Culture of BJ Foreskin Fibroblast Cells

BJ human foreskin fibroblast cells (ATCC™ Cat. # CRL-2522, Bethesda, Md.) were maintained in DME+10% FCS, 2 mM glutamine, 1 mM sodium pyruvate, 0.1 mM MEM nonessential amino acids, 100 U/ml penicillin, and 100 μg/ml streptomycin.

### Example 1.1.7

### Cell-based Assay for Measuring IL-17F/IL-17A Biological Activity

BJ cells were released from the culture flasks using trypsin/EDTA and seeded at 5×10³ cells/well into 96-well microtiter plates, in which hIL-17A, hIL-17F, or hIL-17F/IL-17A had been prediluted in culture medium with or without soluble receptors. In treatments in which antibodies to cell-surface receptors were used, cells were seeded into wells containing antibody before the cytokine was added. Cells were incubated at 37° C. for 16-24 hours, and then supernatants were removed and analyzed for GRO-α by ELISA (matched antibody pairs (MAB275 for capture, BAF275 for detection), R&D Systems, Minneapolis, Minn.).

### Example 1.1.8

### Human IL-17R.Fc and Human IL-17RC.Fc Overexpression in HEK293 Cells

HEK293 cells were transduced to overexpress either human IL-17R.Fc (hIL-17RC.Fc) or human IL-17RC.Fc (hIL-17RC.Fc) using retrovirus supernatants generated from transient transfections of 293 VSV-G cells. Briefly, 293 VSV-G cells plated in 10 mm culture dishes were transfected with 6 μg retroviral plasmid containing either hIL-17R.Fc or hIL-17RC.Fc using 9 μl FUGENE® 6 according to manufacturer's instruction (Roche, Indianapolis, Ind.). After 24 hours incubation at 37° C., the transfection medium was removed and replaced with 6 ml drug-free medium and the culture dishes were incubated at 32° C. Viral supernatants were collected at 48 hours and subsequently at 14-24 hour intervals for 3 days. Supernatants were frozen at −80° C. immediately after collection. The HEK293 cells were plated in a 6-well culture plate one day prior to transduction. The culture medium was aspirated and replaced with 2 ml freshly thawed retrovirus supernatant containing 6 μg/ml polybrene. The plate was centrifuged at 730×g, 32° C. for 1 hour, and then returned to the 37° C. incubator. After 6 hours, 3 ml of culture medium was added to the viral supernatant in each well. The transduced cells were expanded into larger culture dishes the following day.

### Example 1.1.9

### siRNA Transfections

BJ fibroblast cells were seeded in culture medium at 10⁴ cells/well in 96-well plates one day prior to transfection. BJ cells were transfected with DHARMAFECT® #1 transfection reagent according to manufacture's instructions (Cat. # T-2001-03, Dharmacon, Lafayette, Colo.). A mixture of 20 nM of siRNA diluted into 10 μl with OPTI-MEM® (Invitrogen, Carlsbad, Calif.) and preincubated for 5 minutes at room temperature was combined with a mixture of 0.3 μl of DHARMAFECT® #1 added to 9.7 μl of OPTI-MEM®, mixed well, and incubated for 20 min at room temperature; 20 μl of transfection mix was then added to each well of cells containing 80 μl of culture medium. After 24 hours, the transfection medium was removed and replaced with culture medium containing hIL-17F, hIL-17A or hIL-17F/IL-17A at various concentrations. Supernatants were collected at 16 hours and the cells were washed once with PBS and analyzed for GRO-α by ELISA (matched antibody pairs, R&D Systems).

### Example 1.1.10

### Quantification of siRNA-Mediated Degradation of Target mRNAs

The TURBOCAPTURE® mRNA kit (Qiagen) was used to isolate mRNA from BJ fibroblast cells according to manufacturer's instructions. A one-step Eurogentec RTqPCR masterMix Plus, TAQMAN® protocol was used whereby 10 μl of mRNA per sample was used in 25 μl TAQMAN® PCR reactions performed on an ABI Prism 7700 DNA Sequence Detector (Applied Biosystems, Foster City, Calif.). The conditions for TAQMAN® PCR were as follows: 30 minutes at 48° C., 10 minutes at 95° C., then 40 cycles each of 15 seconds at 95° C. and 1 minute at 60° C. on MicroAmp Optical 96-well plates, covered with MicroAmp Optical caps. Each plate contained triplicates of the test cDNA templates and no-template controls for each reaction mix. The expression for each mouse gene was normalized to human beta 2-microglobulin gene expression. The TAQMAN® gene expression assay probe-primer sets for IL-17R (Hs00234888_m1) and IL-17RC (Hs00262062_m1) were acquired from Applied Biosystems.

### Example 1.1.11

### Western Blot Analysis of siRNA Transfection Efficiency

For Western blot analysis, 1.2×10⁴ HEK293 cells and seeded in 96-well plates were transfected with IL-17R or IL-17RC plasmid using the method described in Example 1.1.9. After 48 hours of transfection, cells were washed once with PBS and lysed on ice using M-PER Mammalian Protein Extraction Reagent (Cat# 78501, Pierce Biotechnology, Inc., Rockford, Ill.). After extraction, protein was then loaded onto an SDS-PAGE gel and transferred to nylon membranes. The membranes were blocked for 30 minutes with 5% nonfat dried milk in PBS with 0.1% Tween20. IL-17R or IL-17RC antibody was added to the membranes at 1:4000 for overnight incubation (anti-human IL-17R antibody, Cat. # AF177, anti-human IL-17RC antibody, Cat. # AF2269, R&D Systems, Minneapolis, Minn.). The membranes were washed three times with PBS with 0.1% Tween 20 for 10 minutes each. Following 1 hour of incubation with the donkey anti-goat IgG-HRP at 1:2000 (Cat. # SC-2020, Santa Cruz Biotechnology Inc, Santa Cruz, Calif.), the proteins were visualized using WESTERN LIGHTING® Western Blot Chemiluminescence Reagent Plus (Cat. # NEL103001EA, Perkin-Elmer, Wellesley, Mass.).

### Example 1.1.12

### Binding Kinetics of IL-17F, IL-17A, or IL-17F/IL-17A Binding with IL-17R or IL-17RC Receptors

A Biacore 2000 instrument (Biacore, Piscataway, N.J.) was used for kinetic measurements. Sensor chip surfaces comprising purified hIL-17R.Fc or hIL-17RC.Fc (Wyeth, Cambridge, Mass.) were prepared using amine coupling according to the manufacturer's recommendation (Biacore). Briefly, the sensor chip surface was first activated by injecting a mixture of N-ethyl-N-(2-dimethylaminopropyl) carbodiimide hydrochloride and N-hydroxysuccinimide (NHS-EDC) (Biacore) over each flow cell. Five μg/ml hIL-17R.Fc or hIL-17RC.Fc in 10 mM sodium acetate at a pH of 4.5 was injected over separate flow cells, with a desired target level of 1000 to 2000 RU. Remaining active sites were blocked by 1 M ethanolamine HCl. A reference surface was prepared with an injection of NHS-EDC followed by 1 M ethanolamine HCl. All experiments were performed at 22° C. and the data collection rate was 10 Hz. Human IL-17F, hIL-17A or hIL-17F/IL-17A heterodimer was each diluted into HBST buffer (10 mM Hepes with 0.15 M NaCl, 3.4 mM EDTA, and 0.005% surfactant P20) at an initial concentration of 400 nM and serially diluted to 100 nM, 50 nM, 25 nM, 12.5 nM, 6.25 nM, 3.12 nM and 1.56 nM in the same buffer. Samples were injected in triplicate over each sensor surface at 50 μL/minute to allow for 3-minute association followed by 10-minute dissociation. The surface was regenerated at the end of each dissociation with a 30-second injection of 30% solution of 1.83 M MgCl₂, 0.46 M KSCN, 0.92 M urea, and 1.83 M guanidine-HCl, followed by two consecutive 15-second HBST injections. Samples were tested at least twice to obtain results from more than one immobilized sensor surface. Data were double referenced as described in Myszka ((1999) J. Mol. Recognit. 12:249-84) to improve data quality using Scrubber2 software (BioLogic Software v2.0a, Campbell, Australia). The resulting kinetic data was fit to a 1:1 binding model using Biacore evaluation software version 3.2.

### Example 1.2

### Results

### Example 1.2.1

### Human IL-17F/IL-17A Binds to Human IL-17R.Fc and Human IL-17RC.Fc

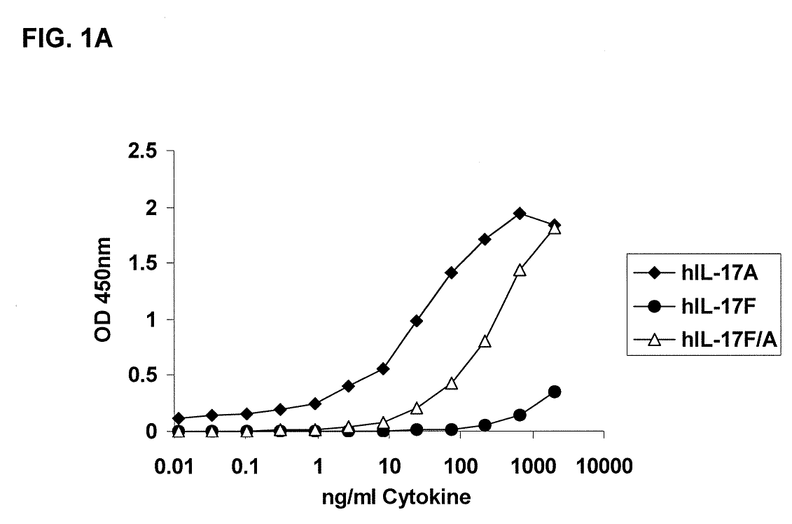

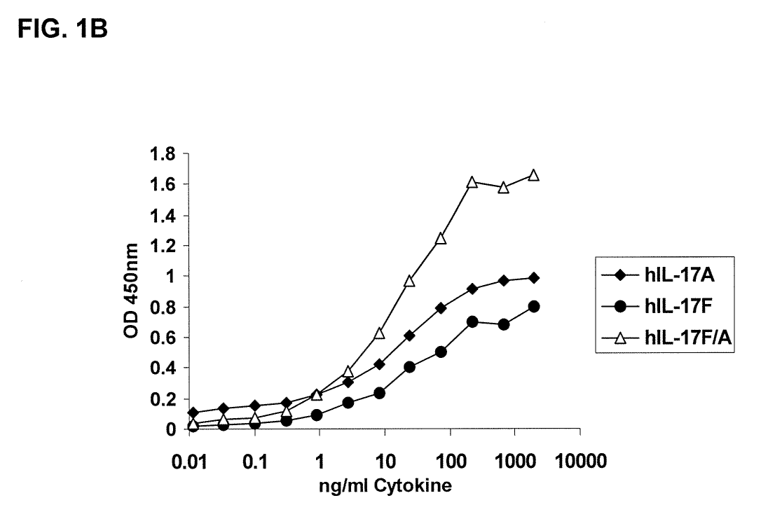

The binding of hIL-17F, hIL-17A or hIL-17F/IL-17A to hIL-17R.Fc and/or hIL-17RC.Fc was evaluated by indirect sandwich ELISA. All three cytokines bound to hIL-17RC.Fc with approximately the same EC₅₀ of 18-25 ng/ml (FIG. 1B). However, the EC₅₀ for the three cytokines were different for binding to hIL-17R.Fc. The tightest binding occurred between hIL-17A and hIL-17R.Fc with an EC₅₀ of 25 ng/ml. Human IL-17F bound weakly to hIL-17R.Fc with an EC₅₀ greater than 2000 ng/ml. The hIL-17F/IL-17A heterodimer had an EC₅₀ of 300 ng/ml, which is 10-fold weaker than the binding of hIL-17A but approximately 10-fold tighter than the binding of hIL-17F (FIG. 1A).

### Example 1.2.2

### Binding Kinetics of Human IL-17F/IL-17A with Human IL-17R.Fc and Human IL-17RC.Fc

The binding kinetics of human IL-17F, hIL-17A and hIL-17F/IL-17A to both hIL-17R.Fc and hIL-17RC.Fc receptors were compared. The association and dissociation rate constants were directly measured by real-time surface plasmon resonance using Biacore, as described in Example 1.1.12. The calculated dissociation constants are shown in Table 3. Human IL-17A exhibited the tightest binding to hIL-17R.Fc with a K_{D} of about 2 nM. In contrast, hIL-17F bound hIL-17R.Fc relatively weakly with a K_{D} of about 174 nM. Interestingly, the hIL-17F/IL-17A heterodimer had a K_{D} value for hIL-17R.Fc of about 26 nM, i.e., intermediate between that for hIL-17A and hIL-17F. These ligands, hIL-17F, hIL-17A, and hIL-17F/IL-17A, each bound hIL-17RC.Fc with similar on and off rates and hence also had similar K_{D} values of about 11-20 nM.

TABLE 3
Kinetic Dissociation Constants of Human IL-17A, Human IL-17F, or Human
IL-17F/IL-17A Binding to Human IL-17RA.Fc and HumanIL-17RC.Fc

| Injected | Immobilized | | | |
|---|---|---|---|---|
| Analyte | Ligand | Kₒₙ (l/M s) | K_{off} (l/s) | K_{D} (M) |
| IL-17A | IL-17RC.Fc | 8.92 ± 0.39 × 10⁴ | 1.79 ± 0.08 × 10⁻³ | 2.01 ± 0.18 × 10⁻⁸ |
| IL-17F | IL-17RC.Fc | 1.28 ± 0.07 × 10⁵ | 2.12 ± 0.20 × 10⁻³ | 1.66 ± 0.06 × 10⁻⁸ |
| IL-17F/A | IL-17RC.Fc | 1.44 ± 0.15 × 10⁵ | 1.51 ± 0.16 × 10⁻³ | 1.06 ± 0.22 × 10⁻⁸ |
| IL-17A | IL-17R.Fc | 1.39 ± 0.15 × 10⁵ | 2.94 ± 0.70 × 10⁻⁴ | 2.15 ± 0.73 × 10⁻⁹ |
| IL-17F | IL-17R.Fc | 9.43 ± 0.38 × 10³ | 1.64 ± 0.10 × 10⁻³ | 1.74 ± 0.07 × 10⁻⁷ |
| IL-17F/A | IL-17R.Fc | 4.28 ± 1.46 × 10⁴ | 1.03 ± 0.01 × 10⁻³ | 2.55 ± 0.83 × 10⁻⁸ |

### Example 1.2.3

### Biological Activity of Human IL-17F/IL-17A

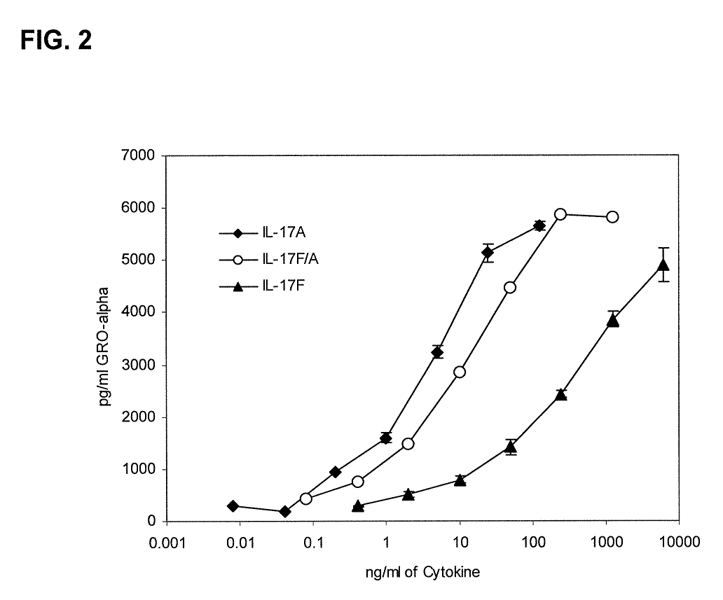

The biological activity of hIL-17F/IL-17A was evaluated using a cell-based assay. ELISA analysis of conditioned medium from BJ cells cultured with hIL-17F, hIL-17A, or hIL-17F/IL-17A heterodimer showed that all three cytokines induced GRO-α secretion in BJ cells, and that hIL-17F was less potent than hIL-17A. The hIL-17F/IL-17A heterodimer was found to be a more potent inducer of GRO-A production by BJ cells compared to hIL-17F but not hIL-17A (FIG. 2). Similar results were obtained when cell lines other than BJ cells were used (data not shown).

### Example 1.2.4

### Effect of Human IL-17R.Fc and Human IL-17RC.Fc on the Biological Activity of Human IL-17F/IL-17A

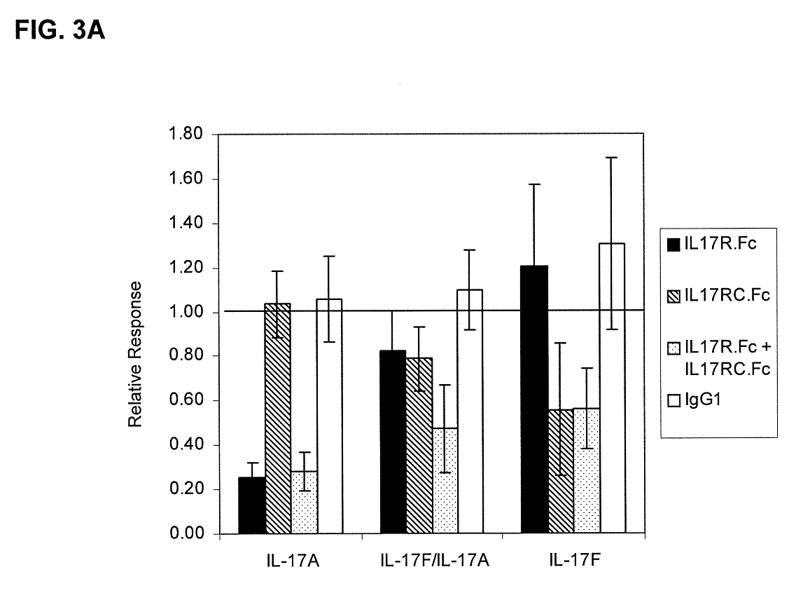

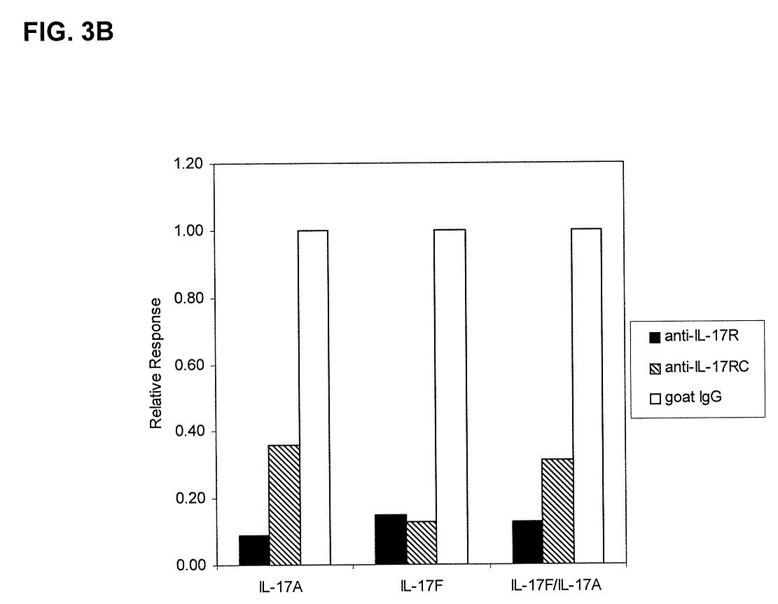

In order to evaluate whether the observed biological activities for hIL-17F, hIL-7A and hIL-17F/IL-17A are due to interactions with either or both of the two proposed receptors, the activities of the cytokines were measured in the presence and absence of the soluble receptors, hIL-17R.Fc and hIL-17RC.Fc. As shown in FIG. 3A, the activity of hIL-17A was decreased almost to background in the presence of hIL-17R.Fc, while no significant effect on IL-17A activity was observed in the presence of IL-17RC.Fc receptor in this experiment. Treatment with both IL-17R.Fc and IL-17RC.Fc did not increase the inhibition of IL-17A activity over that of the IL-17R.Fc alone. The activity of hIL-17F was blocked in the presence of IL-17RC.Fc receptor, but not IL-17R.Fc receptor; while addition of both IL-17R.Fc and IL-17RC.Fc did not increase inhibition of IL-17F activity over that of IL-17RC.Fc alone. In contrast, although both IL-17R.Fc and IL-17RC.Fc had some inhibitory effect on the activity of the IL-17F/IL-17A heterodimer, the combination of the two soluble receptors had an additive effect, significantly blocking the activity of the heterodimer. Nonspecific human IgG had no effect on the activity of any of the three cytokines. These data indicate that while soluble IL-17R can inhibit the activity of IL-17A and soluble IL-17RC can inhibit the activity of IL-17F; the combination of the two soluble receptors is necessary for a significant effect on IL-17F/IL-17A heterodimer activity.

### Example 1.2.5

### Effect of Anti IL-17R and Anti-IL-17RC Antibodies on the Biological Activity of Human IL-17F/IL-17A

The activities of hIL-17F, hIL-17A and hIL-17F/IL-17A were also evaluated using BJ cells preincubated with and without anti-human IL-17R or anti-human IL-17RC antibodies. As shown in FIG. 3B, the activities of the hIL-17 cytokines were decreased significantly when the BJ cells were treated with anti-human IL-17R antibody. However, anti-human IL-17RC antibody had a more profound effect on the activity of hIL-17F compared to the activities of hIL-17A and hIL-17F/IL-17A. The ability of the antibodies to neutralize activity of these molecules is in direct contrast to that observed using the soluble receptors.

### Example 1.2.6

### Effect of Human IL-17R and Human IL-17RC on the Biological Activity of Human IL-17F/IL-17A

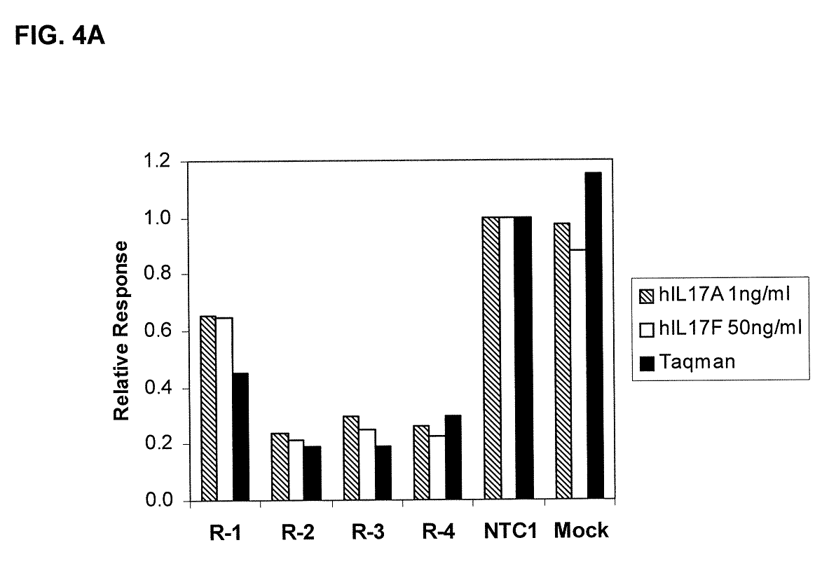

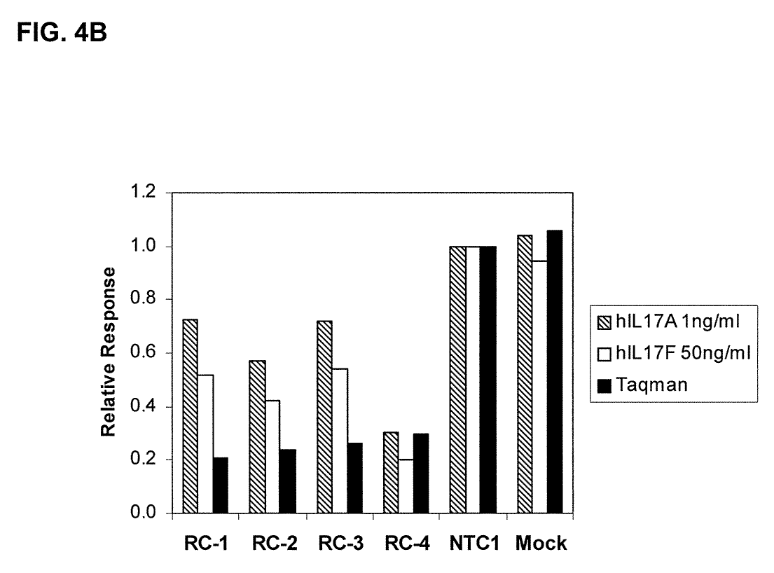

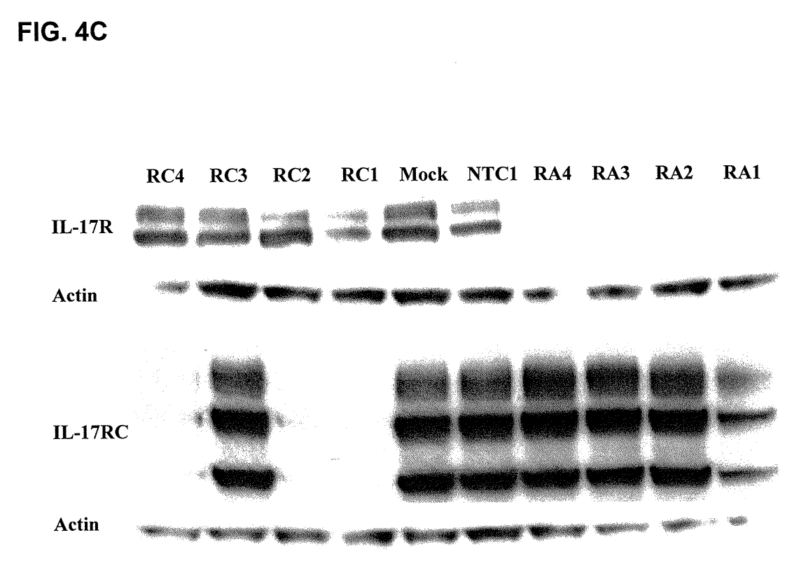

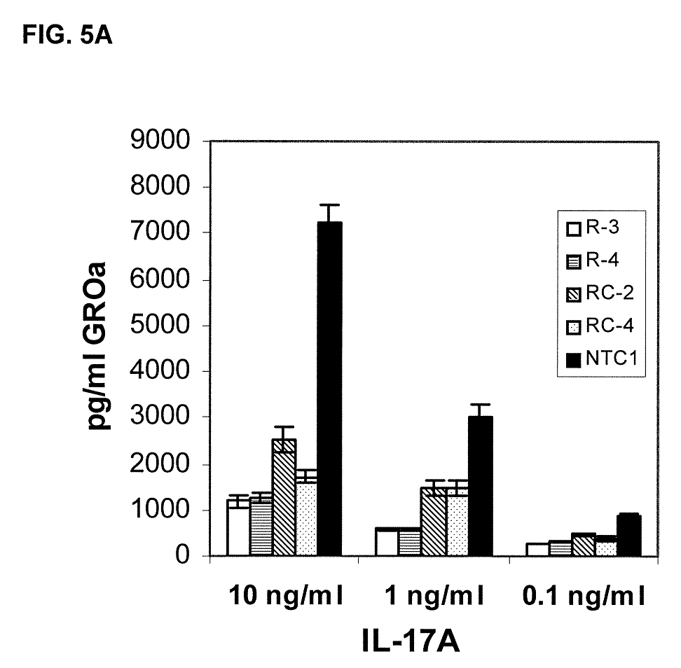

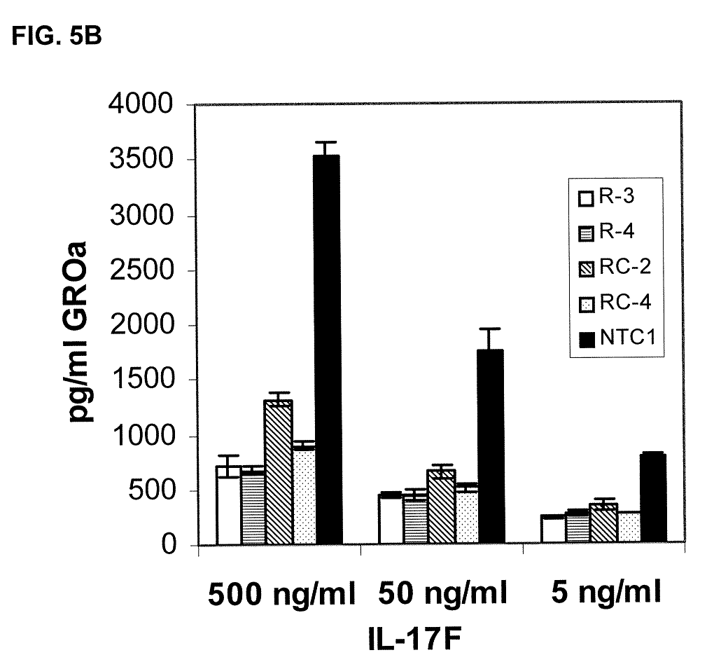

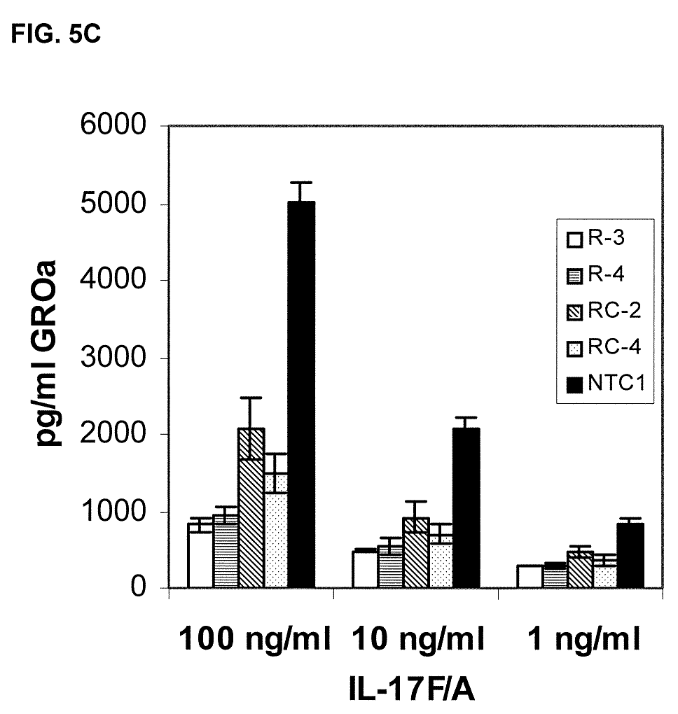

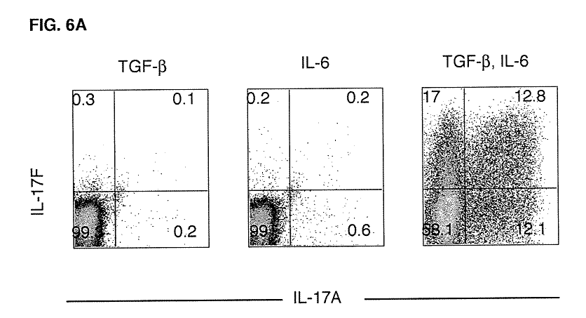

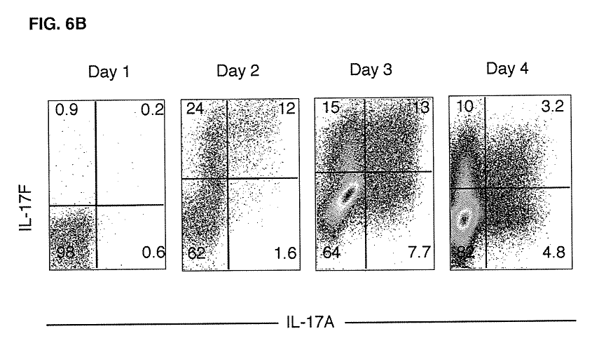

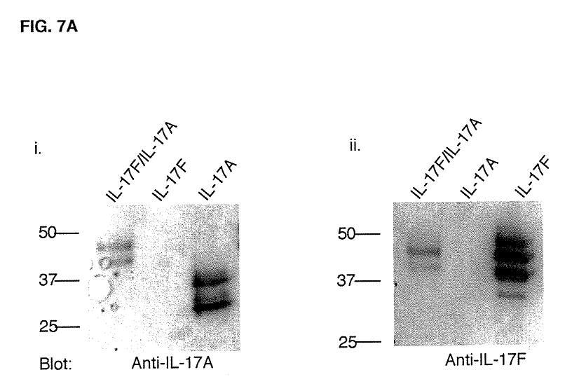

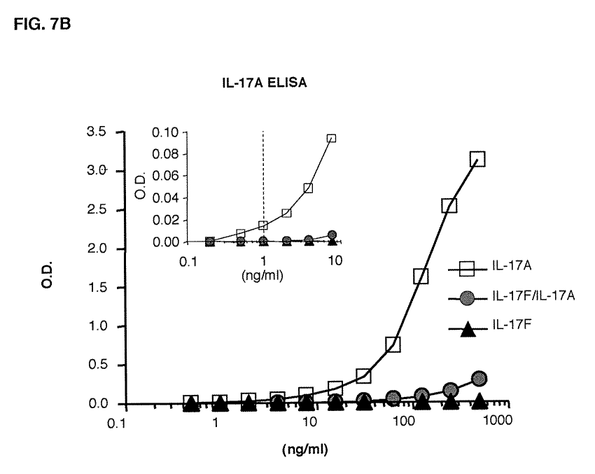

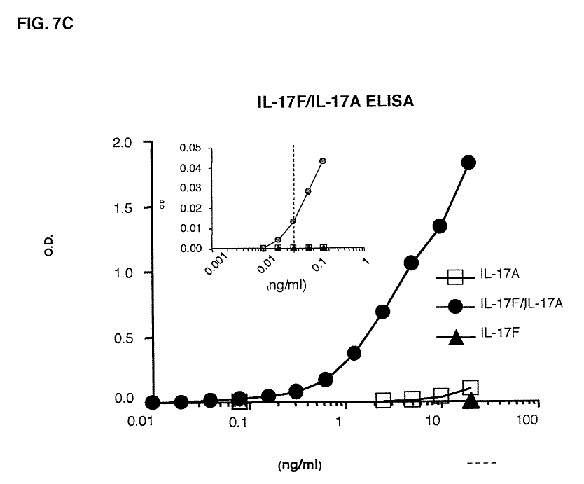

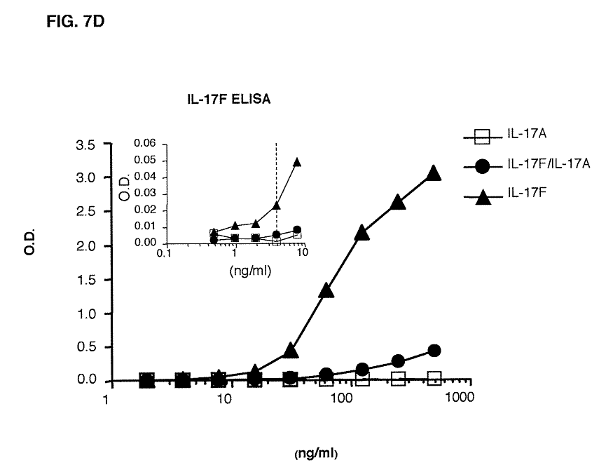

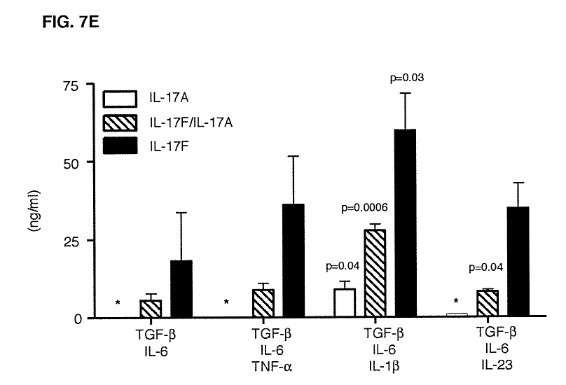

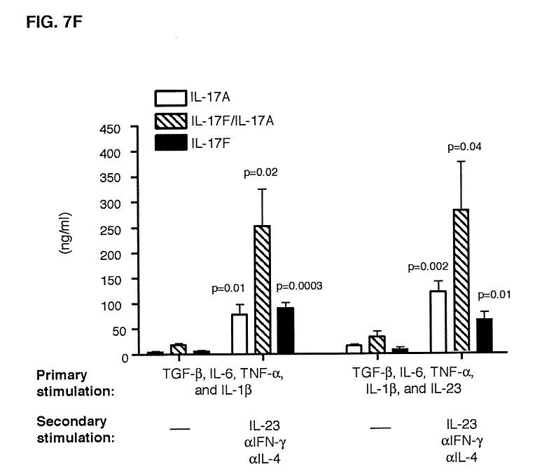

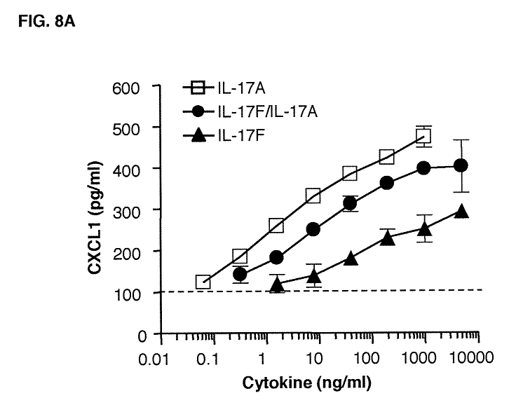

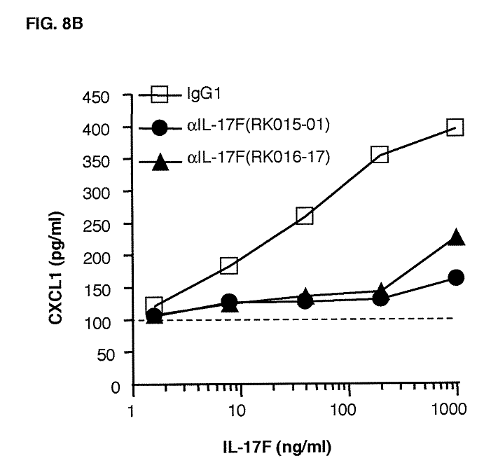

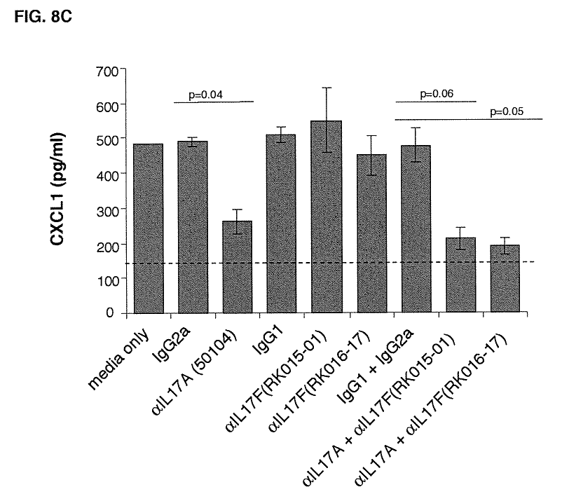

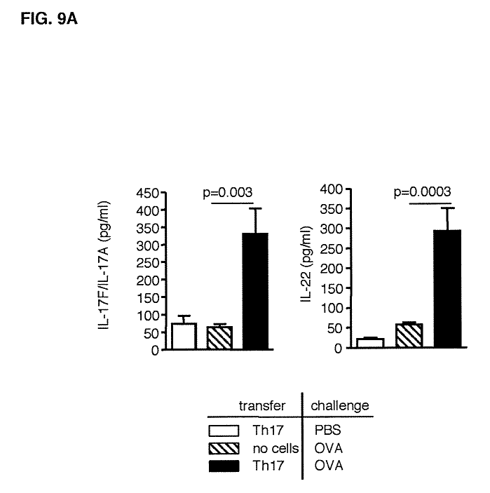

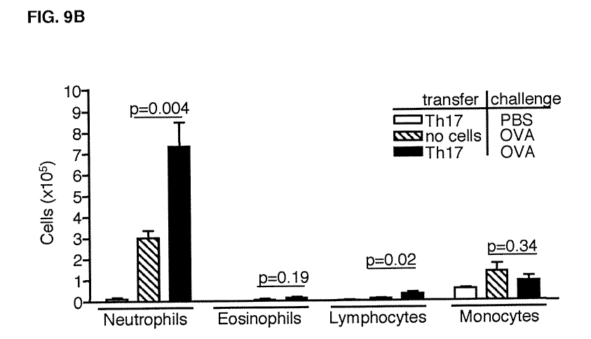

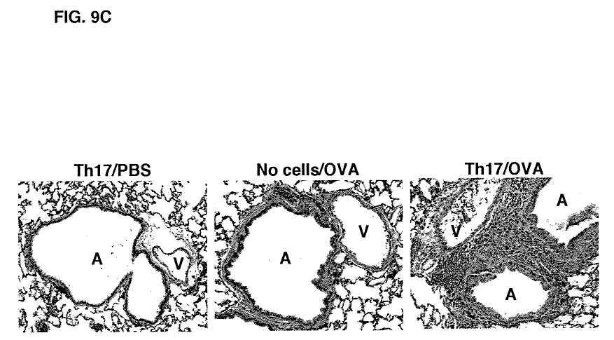

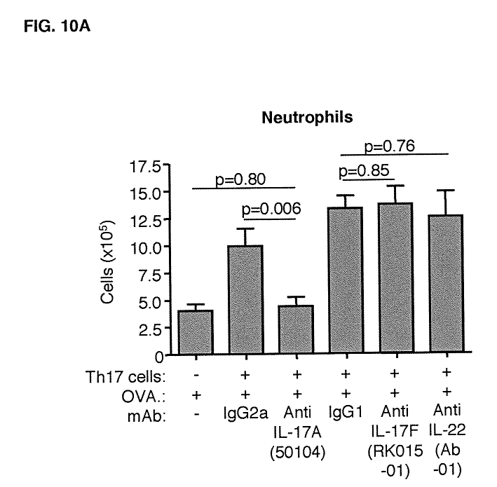

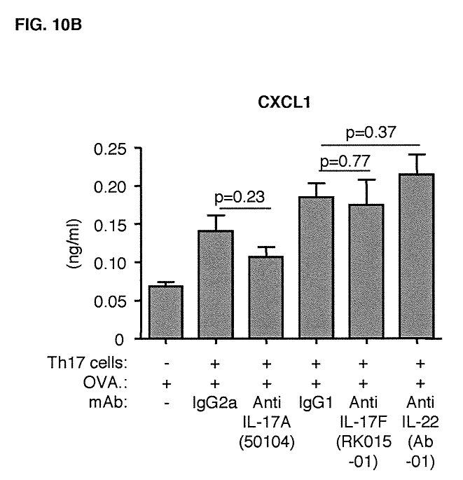

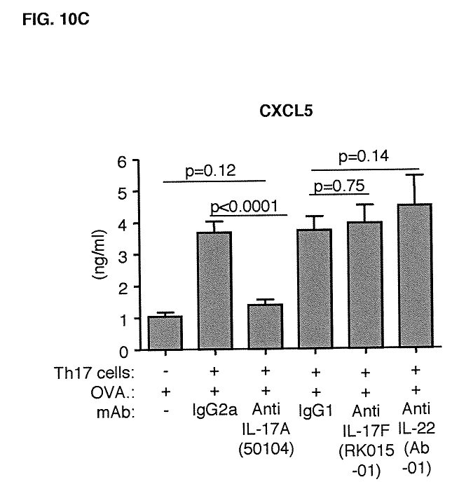

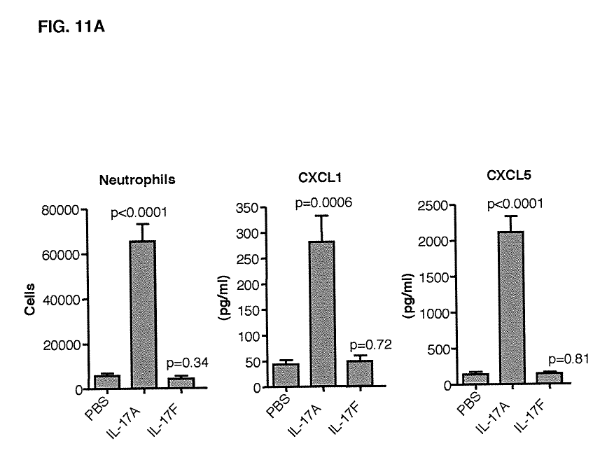

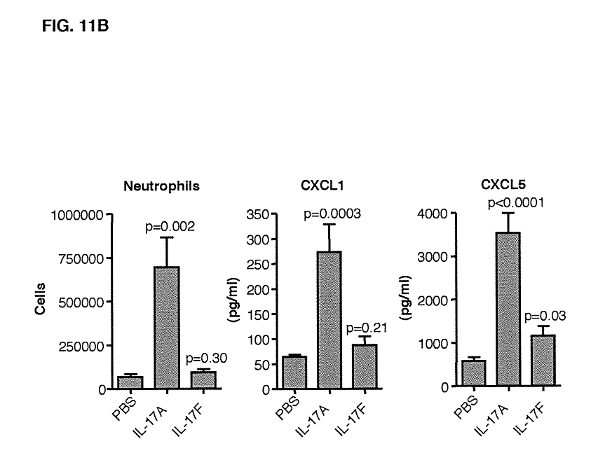

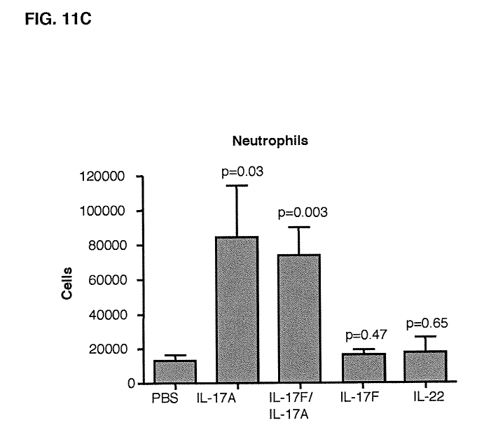

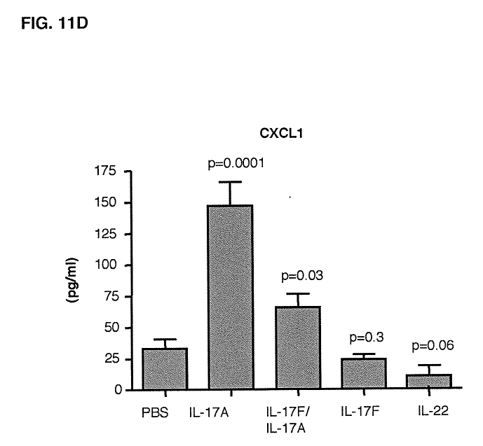

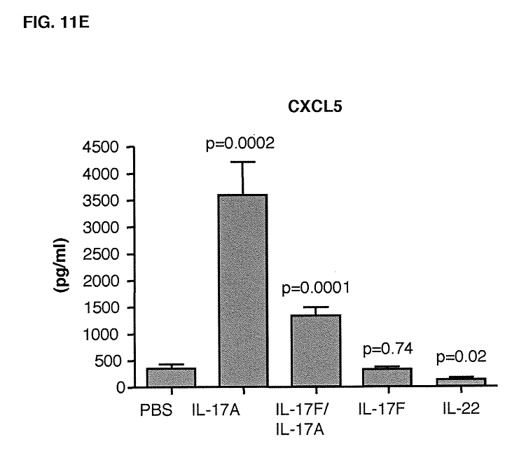

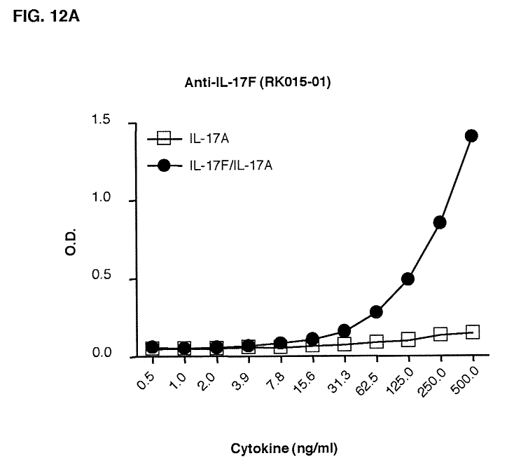

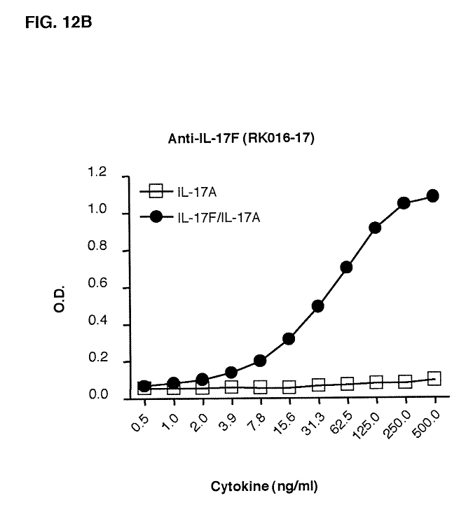

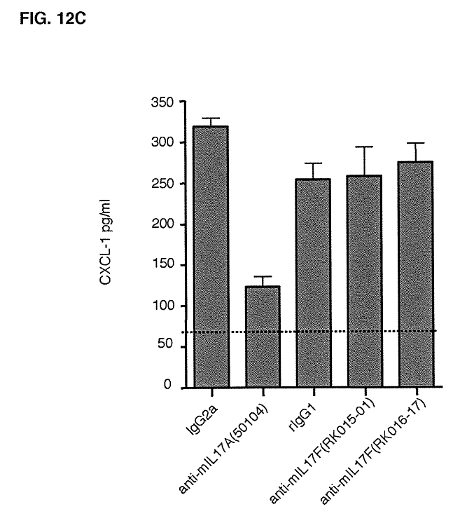

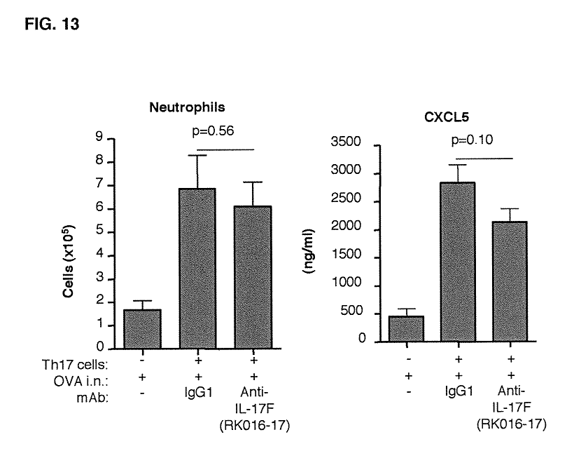

The role of hIL-17R and hIL-17RC in hIL-17F, hIL-17A or hIL-17F/IL-17A signaling was further evaluated. BJ cells were transfected with different hIL-17R or hIL-17RC siRNAs followed by the addition of either hIL-17F or hIL-17A, and the relative responses were determined by ELISA (FIG. 4). The two best siRNA oligos for hIL-17R(R-3 and R-4) or hIL-17RC (RC-2 and RC-4) from the oligos evaluated for each receptor were selected based on TAQMAN® results (FIGS. 4A and 4B) and the ability to decrease protein levels in HEK293 cells (FIG. 4C). Human IL-17F, hIL-17A and hIL-17F/IL-17A at three different concentrations were added to BJ cells transfected with either hIL-17R or hIL-17RC siRNAs (FIG. 5). IL-17R and IL-17RC siRNAs decreased the amount of GRO-α secretion for hIL-17F, hIL-17A and hIL-17F/IL-17A at three different concentrations. The IL-17R siRNAs had a greater effect on cytokine activity compared to IL-17RC siRNAs. This result suggests that all three hIL-17 cytokines are dependent upon hIL-17R and hIL-17RC for their activity.

### Example 2

### A Mouse IL-17F/IL-17A Heterodimer Protein is Produced by Mouse Th17 Cells and Induces Airway Neutrophil Recruitment

### Example 2.1

### Materials and Methods

### Example 2.1.1

### Antibodies and Reagents

Anti-mouse IL-17A antibodies (Cat. # 50101, 50104) were obtained from R&D Systems. Anti-mouse IL-17F antibodies (RK015-01, RK016-17), anti-mouse IL-22 antibody (Ab-01), and relevant isotype control antibodies were generated using methods previously described (Liang et al. (2006) supra). Mouse IL-6, mIL-1β, mTNF-α, and mIL-23 were obtained from R&D Systems. mTGF-β and ovalbumin (OVA) were obtained from Sigma (St. Louis, Mo.). OVA₃₂₃₋₃₃₉ was obtained from New England Peptide (Gardner, Mass.). Anti-IFN-γ (Cat. # XMG1.2) and anti-IL-4 (Cat. # BVD4-1D11) were obtained from BD Pharmingen (Franklin Lakes, N.J.).

### Example 2.1.2

### Generation and Purification of mIL-17A, mIL-17F/IL-17A, and mIL-17F

Sequences for recombinant proteins were engineered into expression vectors using conventional methods as previously described (Li et al. (2004) Int. Immunopharmacol. 4:693-708). His-tagged mIL-17A or His-tagged mIL-17F produced in CHO cells was purified over a Nickel NTA Superflow column (Qiagen). The protein was eluted with 250 mM imidazole and further purified by gel filtration (Superdex200, Amersham) to remove any high molecular weight proteins. The purified cytokines were then digested with enterokinase (at a 1500:1 ratio of cytokine to enterokinase) for 4 hours at room temperature. The digested protein was reapplied to Nickel NTA to remove the enterokinase-His-tag.

Mouse IL-17F/IL-17A heterodimer was produced by transient cotransfection of HEK293 cells with equal amounts of plasmid encoding Flag-tagged mIL-17A or HPC (heavy chain of protein C) His-tagged mIL-17F (Lichty et al. (2005) Protein Expr. Purif. 41:98-105). The conditioned medium was harvested 72 hours later and batch bound to an anti-Flag M2 affinity resin (Sigma). The bound proteins (mIL-17A and mIL-17F/IL-17A) were eluted with 200 μg/ml of Flag peptide (Sigma). Mouse IL-17F/IL-17A was then purified from mIL-17A by batch binding to anti-Protein C affinity matrix (Roche). Mouse IL-17F/IL-17A was eluted with 5 mM EDTA, dialyzed against PBS (pH 7.2), and then characterized by SDS-PAGE gel, Western blot analysis, mass spectrometry and analytical size exclusion chromatography. The resulting mIL-17F/IL-17A heterodimer was greater than 99% pure as determined by silver stain analysis. Endotoxin levels for all recombinant proteins are less than 3 EU/mg.

For Western blot analysis, 35 ng of IL-17A, IL-17F/IL-17A, or IL-17F was loaded. Mouse IL-17A was detected by probing with goat anti-mouse IL-17A (AF421NA, 1:2000 dilution, R&D Systems) followed by donkey anti-goat HRP (Jackson Immunoresearch, West Grove, Pa.). IL-17F was detected using serum (1:2000 dilution) from rats, previously immunized with mouse IL-17F, that tested positive for IL-17F reactive antibodies, followed by detection with goat anti-rat HRP (Pierce Biotechnology).

### Example 2.1.3

### In Vitro T Cell Activation

CD4⁺ CD62L⁺ naïve DO11 T cells were purified from spleens of DO11.10 mice using MACs positive and negative selection (Miltenyi Biotech, Auburn, Calif.) as previously described (Liang et al. (2006) supra). 2×10⁵ DO11 T cells were activated with 4×10⁶ irradiated splenocytes and 1 μg/ml OVA₃₂₃₋₃₃₉. Cytokines were added at the following concentrations: 1 ng/ml mTGF-β, 20 ng/ml mIL-6, 10 ng/ml mIL-1β, 10 ng/ml mTNF-α, and 10 ng/ml mIL-23. For restimulation, cells were harvested on day 7 of primary activation, rested overnight, and restimulated with irradiated splenocytes, 1 μg/ml OVA₃₂₃₋₃₃₉, 5 ng/ml mIL-2, and in some cases 10 ng/ml mIL-23, 10 μg/ml of anti-IFN-γ, and 10 μg/ml anti-IL-4. Conditioned medium was harvested on day 4 of primary or secondary stimulation. Intracellular cytokine staining was performed by restimulating cells with 50 ng/ml PMA (Sigma), 1 μg/ml ionomycin (Sigma), and GOLGIPLUG® (BD Pharmingen) for 5 hours. Cells were surface stained and permeabilized using CYTOFIX/CYTOPERM® according to manufacturer's directions (BD Pharmingen). Intracellular cytokine staining was performed using anti-IL-17A PE (TC11-18H10) and anti-IL-17F Alexa 647 (RK015-01). All lymphocytes were cultured in RPMI1640 supplemented with 10% FBS, 2 mM L-glutamine, 5 mM HEPES, 100 U/ml Pen-Strep, and 2.5 μM β-mercaptoethanol.

### Example 2.1.4

### ELISAs

To quantitate the mIL-17A homodimer, plates were coated with 2 μg/ml of anti-IL-17A (Cat. # 50101) overnight. After plates were blocked with 1% BSA in PBS, samples were incubated in the plate for 2 hours at room temperature. A biotinylated version of the same anti-IL-17A antibody was then used at 1 μg/ml to specifically detect plate-bound mIL-17A. To quantitate IL-17F homodimer, ELISAs were performed following a similar scheme using anti-IL-17F (RK016-17) as both the capture (2 μg/ml) and detection reagent (1 μg/ml). The limit of detection for the mIL-17A and mIL-17F ELISAs was 1 ng/ml and 4 ng/ml, respectively. The mIL-17F/IL-17A heterodimer ELISA was performed using anti-IL-17A (Cat. # 50101, 2 μg/ml) as the capture antibody and biotinylated goat anti-IL17F polyclonal antibody (Cat. # BAF2057, 200 ng/ml, R&D Systems) as the detection reagent. The limit of detection for the mIL-17F/IL-17A heterodimer ELISA was 40 pg/ml. To quantitate expression of these molecules in T cell conditioned medium, the appropriate dilutions to determine the amount of mIL-17F/IL-17A heterodimer were made first because this ELISA was the most sensitive. mIL-17A and mIL-17F using the appropriate ELISA were next quantitated. To correct for possible contributions of mIL-17F/IL-17A on each homodimer ELISA, the amount of mIL-17F/IL-17A heterodimer obtained from ELISA for each sample was utilized to back-calculate the amount of O.D. contributed by mIL-17F/IL-17A based on a titration of recombinant mIL-17F/IL-17A on the homodimer ELISAs. This IL-17F/IL-17A O.D. contribution from the actual O.D. value obtained for each sample on the homodimer ELISAs was subtracted before calculating the amount of homodimer present. IL-22 ELISA was performed as previously described (Liang et al. (2006) J. Exp. Med. 203:2271-79). CXCL1 and CXCL5 were quantitated using DuoSet ELISAs following the manufacturer's directions (R&D Systems).

### Example 2.1.5

### Treatment of MLE-12 Cells

2.5×10⁴ MLE-12 cells (ATCC Cat. # CRL-2110) were treated with cytokine, or preincubated combinations of both cytokine and antibody, for 24 hours in a 96-well plate. Conditioned medium was harvested at 24 hours. MLE cells were grown in HITES, 2% FBS, and 2 mM L-glutamine.

### Example 2.1.6

### Animal Experiments

BALB/cByJ and C.Cg-Tg (DO11.10)10Dlo/J (DO11) mice were obtained from Jackson Laboratories (Bar Harbor, Me.). CD4⁺ CD62L⁺ T cells from DO11 mice were differentiated to Th17 cells as described in Example 2.1.3 in the presence of TGF-β, IL-6, IL-1β, TNF-α, and IL-23 for 5 days. To establish Th17-mediated airway inflammation, 2.5×10⁶ Th17 cells were transferred intravenously into naïve BALB/c recipient mice (day 0). Mice were rested for 24 hours and then challenged with 75 μg of OVA intranasally daily for 3 consecutive days (day 1, 2, and 3). Control mice either received Th17 cells and intranasal PBS or just intranasal OVA and no cells. For studies with antibodies, 300 μg of antibody was injected i.p. 1 hour before the first OVA challenge on day 1. Antibody (100 μg) was also administered intranasally 1 hour before each intranasal challenge with OVA on days 1, 2, and 3. Twenty-four hours after the last challenge, the mice were sacrificed and the bronchoalveolar lavage (BAL) was performed using three 0.7 ml washes with PBS. The first of the three lavages was saved for chemokine analysis after the cells were recovered by centrifugation. To determine total cell counts, cells from all three lavages were combined and counted using a CellDyn hematology analyzer (Abbott Diagnostic, Abbott Park, Ill.). Differential cell analysis was performed by counting cytospin slides stained with Hema-3 stain. 200 cells were counted for each slide. Lungs from mice that had not undergone BAL were fixed in 10% neutral buffered formalin for histological analysis. For intranasal studies, BALB/cByJ mice were challenged intranasally with 1.5 μg of recombinant mouse IL-17A, mIL-17F, mIL-17F/IL-17A, or mIL-22 in 75 μl, either once or daily for three consecutive days. 24 hours after challenge, BAL fluid was harvested and analyzed as described above. All mice were used between 8-12 weeks of age and were housed in strict accordance to Wyeth Research IACUC regulations.

### Example 2.1.7

### Data Analysis

All statistical significance values were determined by an unpaired Student's T-test.

### Example 2.2

### Results

### Example 2.2.1

### Mouse IL-17A and Mouse IL-17F Are Coexpressed by Mouse Th17 Cells

Th17 differentiation from naïve T cells is initiated primarily by the combination of TGF-β and IL-6, although other proinflammatory cytokines, such as TNF-α and IL-1β, can further augment the response (Veldhoen et al. (2006) supra; Bettelli et al. (2006) supra; Mangan et al. (2006) supra). Although these studies have definitively shown that IL-17A protein expression is regulated in this fashion, it has not been reported whether IL-17F protein expression is regulated similarly. To examine the regulation of IL-17F expression, naïve (CD4⁺ CD62L⁺) T cells purified from DO11.10 (DO11) mice were activated with irradiated splenocytes, OVA₃₂₃₋₃₃₉, and various cytokines and intracellular cytokine staining for IL-17F was performed. Similar to IL-17A, substantial IL-17F expression was detected with the addition of both TGF-β and IL-6 (FIG. 6A). The relative expression of mIL-17A and mIL-17F during Th17 differentiation was analyzed at several time points after activation. Overall, the expression of mIL-17F was consistently greater than mIL-17A, with expression of both cytokines decreasing after day 3 (FIG. 6B). IL-17A⁺IL-17F⁺ cells represented a substantial portion of Th17 cells, with mIL-17A being highly coexpressed with mIL-17F on day 2 (88% of IL-17A⁺ cells also expressed mIL-17F). The decreased coexpression on day 3 (65%) and day 4 (40%) may be related to overall decreases in mIL-17A and mIL-17F expression. These data demonstrate that IL-17F protein is induced by the combination of TGF-β and IL-6. Furthermore, IL-17A⁺IL-17F⁺ cells represent a substantial population of Th17 cells.

### Example 2.2.2

### Mouse Th17 Cells Produce a Heterodimer Protein Composed of Mouse IL-17A and Mouse IL-17F

Human IL-17F, and presumably hIL-17A, exists as a homodimer held together by a conserved cysteine disulfide bridge (Hymowitz et al. (2001) supra; Wright et al. (2007) supra). The conserved position of these cysteines in mIL-17F and mIL-17A, along with the coexpression of mIL-17A and mIL-17F by mouse Th17 cells, suggests that mIL-7A and mIL-17F may also form a heterodimer. To explore this possibility, recombinant mouse mIL-17F/IL-17A heterodimer was generated by overexpressing differentially tagged versions of mIL-17A and mIL-17F in HEK293 cells. Purification of the putative mIL-17F/IL-17A protein was achieved by sequential purifications using protein tags. Western blot analysis on a nonreducing gel revealed that this purified double-tagged protein contained both mIL-17A and mIL-7F epitopes in the same bands (FIG. 7A). The distinct bands represented differentially glycosylated species (data not shown). These data demonstrate the formation of mIL-17F/IL-17A heterodimers when the mIL-17A and mIL-17F genes are overexpressed in vitro.

To determine if mIL-17F/IL-17A heterodimer is produced by mouse T cells, ELISAs to quantitate mIL-17A, mIL-17F, and mIL-17F/IL-17A were established first. To specifically quantitate homodimers, the same monoclonal antibody was used as both the capture and detection reagent in a sandwich ELISA. This format allows for a successful sandwich to be formed only by homodimers or higher multimers. To quantitate mIL-17F/IL-17A heterodimer, a sandwich ELISA was performed using a mIL-17A specific antibody as the capture reagent in combination with a mIL-17F specific antibody as the detection reagent. The specificity of these ELISAs was validated using purified recombinant mIL-17A, mIL-17F/IL-17A, and mIL-17F proteins (FIGS. 7B-7D).

The amounts of natural mIL-17A, mIL-17F, and mIL-17F/IL-17A produced by Th17 cells activated under different conditions were quantitated. Naïve DO11 T cells were activated with mTGF-β and mIL-6 and in some cases further supplemented with mTNF-α, mIL-1, mIL-23, or all three cytokines. Under all these conditions, mIL-17F was produced at the greatest abundance, with mIL-17F/IL-17A heterodimer having intermediate expression and mIL-17A being expressed in the lowest amount (FIG. 7E). Mouse IL-17A was below the limit of detection (1 ng/ml) in cells activated with the combination of mTGF-β and mIL-6, or when this condition was further supplemented with mTNF-α or mIL-23 (FIG. 7E). Addition of mIL-1β increased mIL-17A expression by 9-fold, mIL-17F/IL-17A by 5-fold, and mIL-17F by 3-fold. IL-23 enhanced mIL-17F/IL-17A production by 1.8-fold and IL-17F production by 2-fold. In contrast, addition of mTNF-α or IL-23 enhanced expression of mIL-17F/IL-17A and mIL-17F modestly, if at all (see, e.g., FIG. 7E). These data demonstrate that naïve T cells differentiated under various Th17 differentiation conditions produce three distinct IL-17 proteins, with IL-17F expressed in highest amounts, followed by IL-17F/IL-17A and then IL-17A.

To examine the expression of these proteins by differentiated Th17 cells, naïve DO11 T cells were first activated in a primary activation for seven days with the indicated cytokines (FIG. 7F). After resting them overnight, these cells were restimulated in the presence of mIL-2 and OVA₃₂₃₋₃₃₉ or with mIL-2, OVA₃₂₃₋₃₃₉, mIL-23, and antibodies to IFN-γ and IL-4. In contrast to the expression profile of naïve cells, mIL-17A, mIL-17F/IL-17A, and mIL-17F were produced in comparable amounts by differentiated Th17 cells restimulated with OVA₃₂₃₋₃₃₉ (FIG. 7F). Mouse IL-23, along with antibodies to IFN-γ and IL-4, considerably enhanced expression of all three proteins, with mIL-7F/IL-17A consistently being produced in higher amounts than mIL-17A or mIL-17F. These data demonstrate that IL-17A expression is elevated in differentiated Th17 cells as compared to newly activated naïve cells. Furthermore, IL-17F/IL-17A heterodimer was expressed by both naïve T cells stimulated with IL-17-inducing conditions and by differentiated Th17 cells.

### Example 2.2.3

### IL-17F/IL-17A Heterodimer is a Biologically Active Protein

IL-17A and IL-17F are known to enhance the expression of chemokines by epithelial cells and fibroblasts, although a direct comparison using mouse cytokines has not been reported. To examine the activity of mIL-17A, mIL-17F/IL-17A, and mIL-17F, a mouse lung epithelial cell line, MLE-12, was treated with these cytokines and the expression of the neutrophil chemoattractant, CXCL1 (KC), was examined. Mouse IL-17A and mIL-17F both enhanced the expression of CXCL1, although mIL-17F was 100-1000 fold less active than mIL-17A (FIG. 8A). The mIL-17F/IL-17A heterodimer also enhanced CXCL1 and was consistently less potent than mIL-17A and more potent than mIL-17F (FIG. 8A). These data demonstrate that mIL-17F/IL-17A is a biologically active protein.

To explore the relative contributions of mIL-17A and mIL-17F in mIL-17F/IL-17A activity, neutralizing antibodies to IL-17F were generated. Two antibodies that completely neutralized the activity of up to 200 ng/ml of mIL-17F on MLE-12 cells with 50 μg/ml of antibody were identified (FIG. 8B). These anti-IL-17F antibodies do not bind or neutralize mIL-17A and can bind to mIL-17F/IL-17A heterodimer (see FIGS. 12A and 12B). An mIL-17A-specific antibody (50104) was also tested and determined able to neutralize the effects of mIL-17A (FIG. 12C), and not mIL-17F (data not shown) on MLE-12 cells. The effects of these antibodies on neutralizing mIL-17F/IL-17A heterodimer were examined. MLE-12 cells were treated with 200 ng/ml of mIL-17F/IL-17A heterodimer in combination with monoclonal antibodies, used at 80 μg/ml (˜100-fold molar excess). The mIL-17A-specific antibody reduced the effects of mIL-17F/IL-17A by ˜85% as compared to its isotype control (IgG2a) (FIG. 8C). In contrast, neutralization of mIL-17F/IL-17A with anti-IL-17F (RK015-01 or RK016-17) had no or only modest effects (up to 20% in some experiments) as compared to the isotype control (IgG1). When an IL-17F-specific antibody was used in combination with an IL-17A-specific antibody, the activity of mIL-17F/IL-17A was almost completely neutralized (˜95%). These data demonstrate that although the combination of an IL-17A-specific antibody and an IL-17F-specific antibody is needed to completely neutralize IL-17F/IL-17A, the majority of the activity of this cytokine can be neutralized using only an IL-17A-specific antibody.

Interpretation of the data presented herein is dependent on whether the mIL-17F-specific antibodies are successfully blocking an interaction between mIL-17F/IL-17A and its receptor(s). If the mIL-17F-specific antibody is binding to mIL-17F/IL-17A, but not blocking its interaction with the receptor, this suggests that at least one receptor binding site is not conserved between mIL-17F/IL-17A and mIL-17F. This may be due to conformational differences in the receptor-binding sites of mIL-17F/IL-17A and mIL-17F, or to the existence of distinct sites on mIL-17F/mIL-17A that interact with an alternate receptor. These possibilities would allow for receptor interactions even when a mIL-17F-specific antibody is bound. In this model, the data using combinations of antibodies would then suggest that binding of a mIL-17A-specific antibody to mIL-17F/IL-17A may alter the mIL-17F/mIL-17A receptor-binding sites such that the mIL-17F-specific antibodies can now produce neutralization. Alternatively, if the mIL-17F-specific antibody alone is successfully blocking an interaction of mIL-17F/mL-17A with its receptor, then the data indicate that this interaction is not necessary and suggest that binding of other receptor sites on mIL-17F/mL-17A is sufficient for signaling. The inventors observed that mIL-17F-specific antibodies are able to further neutralize in combination with a mIL-17A-specific antibody. This suggests that the receptor-binding site blocked by an mIL-17F-specific antibody delivers a signal that is less potent than the signal neutralized by the mIL-17A-specific antibody.

### Example 2.2.4

### Th17 Cells Induce Neutrophilia in Airways that is Dependent on IL-17A

The in vitro data demonstrate that recombinant mIL-17A is more biologically active than mIL-17F, with mIL-17F/IL-17A being less active than mIL-17A and more active than mIL-17F. To examine the relative contributions of IL-17A- and IL-17F-containing proteins in vivo, a Th17-dependent airway inflammation model was established. CD4⁺ CD62L⁺T cells from DO11 mice were differentiated for 5 days with mTGF-β, mIL-6, mIL-1, mTNF-α, and mIL-23, after which cells were adoptively transferred into a naïve BALB/c host. To induce airway inflammation, mice were subsequently challenged daily with intranasal OVA for three consecutive days. Control mice either received Th17 cells and intranasal PBS or no cells and intranasal OVA. Mouse IL-17A and mIL-17F in the BAL fluid were below the limit of detection (1 ng/ml and 4 ng/ml, respectively) in the homodimer-specific ELISAs (data not shown). Expression of mIL-17F/IL-17A heterodimer was detected above the level of detection (40 pg/ml), and a significant six-fold increase in IL-17F/IL-17A heterodimer in mice transferred with Th17 cells and exposed to OVA as compared to control mice was observed (FIG. 9A). A significant six-fold increase in mIL-22, a cytokine recently described to be expressed by Th17 cells (Liang et al. (2006) supra; Chung et al (2006) Cell Res. 16:902-07; Zheng et al. (2007) Nature 445:648-51), was also detected (FIG. 9A). The expression of mIL-17F/IL-17A and mIL-22 demonstrate that Th17 cells were present and activated in the airways. Cellular inflammation in this model was next examined. Mice receiving Th17 cells and OVA had significantly increased neutrophil and lymphocyte numbers in the BAL fluid as compared to either group of control mice (FIG. 9B). Monocytes and eosinophils were not increased in mice receiving Th17 cells and intranasal OVA (FIG. 9B). Histological analysis of lung tissue also revealed enhanced peribronchial and perivascular inflammation in mice transferred with Th17 cells and exposed to OVA when compared to control groups (FIG. 9C). Neutrophils were a prominent component of the inflammation, similar to results observed in the BAL fluid. Taken together, these data demonstrate that Th17 cells can induce an airway inflammatory response characterized by the recruitment of neutrophils.

Although Th17 cells can induce airway neutrophilia, it is unknown which cytokine is specifically responsible for these effects. To examine this issue, neutralizing antibodies to mIL-17A, mIL-17F, and mIL-22 were administered. Treatment with a mIL-17A-specific antibody (Cat. # 50104) significantly reduced the number of neutrophils as compared to isotype (IgG2a) to levels similar to control mice (FIG. 10A). In contrast, neutralizing antibodies to mIL-17F (RK015-01 or RK016-17) or mIL-22 (Ab-01) did not affect neutrophil numbers (FIG. 10A, FIG. 13). No significant effects were observed on lymphocyte, eosinophil, or monocyte numbers in mice treated with antibodies specific for mIL-17A, mIL-17F, or mIL-22 (data not shown). Although concentrations of CXCL1 were not significantly modulated in any of the treatment groups (FIG. 10B), CXCL5 (LIX), another potent neutrophil chemoattractant (Wuyts et al. (1996) J. Immunol. 157:1736-43; Chandrasekar et al. (2001) Circulation 103:2296-02), was significantly reduced by the IL-17A-specific antibody to concentrations similar to control mice (FIG. 10C). Antibodies specific for mIL-17F or mIL-22 did not alter CXCL5 (FIG. 10C). These data demonstrate that administration of an IL-17A-specific antibody alone was sufficient to prevent Th17 cell-induced airway neutrophilia.

### Example 2.2.5

### Mouse IL-17F/IL-17A Recruits Neutrophils In Vivo

In the Th17-dependent airway inflammation model disclosed herein, the expression of mIL-17A or mIL-17F in the BAL fluid was below the limit of detection. As a result, it could not be shown that mIL-17A or mIL-17F was being expressed in the airways. However, the comparable expression of mIL-17A, mIL-17F/IL-17A, and mIL-17F by differentiated Th17 cells suggested that the heterodimer proteins were present, but below the detection limit. To directly examine the effects of mIL-17A and mIL-17F, 1.5 μg of recombinant protein was administered into the airways either once (FIG. 11A) or daily for three consecutive days (FIG. 11B). Neutrophil recruitment and chemokine production in the BAL fluid 24 hours after the last administration was examined. Human IL-17A significantly increased neutrophils, CXCL1, and CXCL5, either when given once (FIG. 11A) or three times (FIG. 11B). In contrast, mIL-17F did not significantly enhance neutrophil numbers or CXCL1 (FIGS. 11A and 11B). A small and significant increase in CXCL5 was observed only when mIL-17F was given three times (FIG. 11B). Increasing the dose of mIL-17F by ten-fold (15 μg) did not further enhance neutrophils, CXCL1, or CXCL5 relative to what was observed with 1.5 μg, either when given once or three times (data not shown). Recombinant mIL-22 did not result in any observable increase in neutrophils or chemokines when given once (FIGS. 11C-11E). Expression of G-CSF, CXCL2, MCP-1, IL-6, TNF-α, and IFN-γ was not detected in any of these samples (data not shown).

The activity of mIL-17F/IL-17A heterodimer with mIL-17A and mIL-17F in the airways was compared. One dose of 1.5 μg mIL-17F/IL-17A induced a significant increase in neutrophils, CXCL1, and CXCL5 (FIGS. 11C-11E). Although the induction of neutrophils was similar between mIL-17A and mIL-17F/IL-17A (p=0.76), CXCL1 and CXCL5 expression was 2-3 fold less in mice treated with mIL-17F/IL-17A than mIL-17A. These findings show that mIL-17F/IL-17A heterodimer is a biologically active molecule in vivo and can induce the recruitment of neutrophils.

What is claimed is:

1. A method of screening for compounds capable of antagonizing IL-17F/IL-17A signaling comprising the steps of:
(a) contacting a sample containing IL-17F/IL-17A and IL-17R with one of a plurality of test compounds; and
(b) determining whether the biological activity of IL-17F/IL-17A in the sample is decreased relative to the biological activity of IL-17F/IL-17A in a sample not contacted with the test compound,
whereby such a decrease in the biological activity of IL-17F/IL-17A in the sample contacted with the test compound identifies the compound as an IL-17F/IL-17A signaling antagonist.

2. The method of claim 1, further comprising a first or a last step of identifying whether the IL-17F/IL-17A signaling antagonist is a specific IL-17F/IL-17A signaling antagonist.

3. The method of claim 2, wherein the step of identifying comprises the steps of:
(a) contacting a sample containing IL-17A and IL-17R with the IL-17F/IL-17A signaling antagonist;
(b) determining whether the biological activity of IL-17A in the sample is decreased relative to the biological activity of IL-17A in a sample not contacted with the IL-17F/IL-17A signaling antagonist;
(c) contacting a sample containing IL-17F and IL-17R with the IL-17F/IL-17A signaling antagonist; and
(d) determining whether the biological activity of IL-17F in the sample is decreased relative to the biological activity of IL-17F in a sample not contacted with the IL-17F/IL-17A signaling antagonist,
whereby a failure of the IL-17F/IL-17A signaling antagonist to decrease the biological activity of both IL-17F and IL-17A identifies the IL-17F/IL-17A signaling antagonist as a specific IL-17F/IL-17A signaling antagonist.

4. A method of screening for compounds capable of antagonizing IL-17F/IL-17A signaling comprising the steps of:
(a) contacting a sample containing IL-17F/IL-17A and IL-17RC with one of a plurality of test compounds; and
(b) determining whether the biological activity of IL-17F/IL-17A in the sample is decreased relative to the biological activity of IL-17F/IL-17A in a sample not contacted with the test compound,
whereby such a decrease in the biological activity of IL-17F/IL-17A in the sample contacted with the test compound identifies the compound as an IL-17F/IL-17A signaling antagonist.

5. The method of claim 4, further comprising a first or a last step of identifying whether the IL-17F/IL-17A signaling antagonist is a specific IL-17F/IL-17A signaling antagonist.

6. The method of claim 5, wherein the step of identifying comprises the steps of:
(a) contacting a sample containing IL-17A and IL-17RC with the IL-17F/IL-17A signaling antagonist;
(b) determining whether the biological activity of IL-17A in the sample is decreased relative to the biological activity of IL-17A in a sample not contacted with the IL-17F/IL-17A signaling antagonist;
(c) contacting a sample containing IL-17F and IL-17RC with the IL-17F/IL-17A signaling antagonist; and
(d) determining whether the biological activity of IL-17F in the sample is decreased relative to the biological activity of IL-17F in a sample not contacted with the IL-17F/IL-17A signaling antagonist,
whereby the failure of the IL-17F/IL-17A signaling antagonist to decrease the biological activity of both IL-17F and IL-17A identifies the IL-17F/IL-17A signaling antagonist as a specific IL-17F/IL-17A signaling antagonist.

7. A compound identified by the method of claim 1.

8. A method of inhibiting IL-17F/IL-17A biological activity in a subject, the method comprising administering to the subject an IL-17F/IL-17A signaling antagonist.

9. The method of claim 8, wherein the IL-17F/IL-17A biological activity is GRO-A secretion.

10. The method of claim 8, wherein the IL-17F/IL-17A signaling antagonist is selected from the group consisting of an antagonistic small molecule, an antagonistic antibody, an IL-17R fusion polypeptide, and an IL-17RC fusion polypeptide.

11. The method of claim 8, wherein the IL-17F/IL-17A signaling antagonist is a specific IL-17F/IL-17A signaling antagonist.

12. The method of claim 8, wherein the IL-17F/IL-17A signaling antagonist is the compound of claim 7.

13. A method of treating a subject at risk for, or diagnosed with, an IL-17F/IL-17A-associated disorder comprising administering to the subject a therapeutically effective amount of an IL-17F/IL-17A signaling antagonist.

14. The method of claim 13, wherein the IL-17F/IL-17A signaling antagonist is selected from the group consisting of an antagonistic small molecule, an antagonistic antibody, an IL-17R fusion polypeptide, and an IL-17RC fusion polypeptide.

15. The method of claim 13, wherein the IL-17F/IL-17A signaling antagonist is a specific IL-17F/IL-17A signaling antagonist.

16. The method of claim 13, wherein the IL-17F/IL-17A signaling antagonist is the compound of claim 7.

17. A pharmaceutical composition comprising an IL-17F/IL-17A signaling antagonist and a pharmaceutically acceptable carrier.

18. The composition of claim 17, wherein the IL-17F/IL-17A signaling antagonist is selected from the group consisting of an antagonistic small molecule, an antagonistic antibody, an IL-17R fusion polypeptide, and an IL-17RC fusion polypeptide.

19. The composition of claim 17, wherein the IL-17F/IL-17A signaling antagonist is a specific IL-17F/IL-17A signaling antagonist.

20. The composition of claim 17, wherein the IL-17F/IL-17A signaling antagonist is the compound of claim 7.

21. An isolated antibody capable of specifically binding IL-17F/IL-17A heterodimer.

22. The antibody of claim 21, wherein the antibody inhibits IL-17F/IL-17A signaling.

23. A small molecule capable of specifically binding IL-17F/IL-17A heterodimer.

24. The small molecule of claim 23, wherein the small molecule inhibits IL-17F/IL-17A signaling.

25. The method of claim 13, wherein the IL-17F/IL-17A-associated disorder is an inflammatory disorder.

26. The method of claim 13, wherein the IL-17F/IL-17A-associated disorder is a respiratory disorder.

27. The method of claim 26, wherein the respiratory disorder is selected from the group consisting of airway inflammation, asthma, and COPD.

28. A method of inducing airway inflammation in a subject comprising administering to the subject IL-17F/IL-17A.

29. The method of claim 28, wherein the subject is a mouse.


SEQUENCE LISTING (37 sequences)

SEQ ID NO: 1 - DNA, 1883 bp, Homo sapiens
  CDS (54)..(521)
gaattccggcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaatgaactttaagtatgagaaaagttcagcccaagtaaaataaaaactcaatcacattcaattccagagtagtttcaagtttcacatcgtaaccattttcgcccggaattcaaaaaaaaa

SEQ ID NO: 2 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 3 - DNA, 492 bp, Homo sapiens
  CDS (1)..(492)
atgacagtgaagaccctgcatggcccagccatggtcaagtacttgctgctgtcgatattggggcttgcctttctgagtgaggcggcagctcggaaaatccccaaagtaggacatacttttttccaaaagcctgagagttgcccgcctgtgccaggaggtagtatgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcacccctgtcatccaccatgtgcagtaa

SEQ ID NO: 4 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 5 - DNA, 3420 bp, Homo sapiens
  CDS (134)..(2734)
ggctggaagccggaagcgagcaaagtggagccgactcgaactccaccggcacgagggcggaaaagaaagcctcagaacgttcgctcgctgcgtccccagccggggccgagccctccgcgacgccacccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgcggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccgctgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccccgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagctttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacgttaaacgaacaggatgggccgggcacggtggctcacgcctgtaatcccagcacactgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaa

SEQ ID NO: 6 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 7 - DNA, 2691 bp, Homo sapiens
  CDS (219)..(2594)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccggtgagtctggaaccctggggagacgaggaaaggctcagggttcagtttttggctcagcaaagccttagcctggctcctgtcactgctgccactgccagaactgccctgtctggtctgtctggtgctgatggtagaagagaagaacggggaaggggcaagagctgggtctgtctttctctgggagggtctgggaatacggagccccagaaaaagggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 8 - protein, 791 aa, Homo sapiens
MPVPWFLLSLALGRSPVVLSLERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 9 - DNA, 2478 bp, Homo sapiens
  CDS (219)..(2381)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 10 - protein, 720 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 11 - DNA, 2527 bp, Homo sapiens
  CDS (219)..(1835)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcggggggtgagtgggagcaagcgctgggcggagggccgcccccggggagccaggcctgtgccagctcacctcttccctccccatctgttttctccggcagcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 12 - protein, 538 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGGEWEQALGGGPPPGSQACASSPLPSPSVFSGSGRQGPRGSAPLLSR

SEQ ID NO: 13 - DNA, 2584 bp, Homo sapiens
  CDS (219)..(1022)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctggtgaggcctcccccttcccaagtccattcccactgtaggccgatgcctgtgcaaaggacgcagtgccatatcagagaggatccttgaagaggactcaccccaagcaagggaaaattggtgggggaacttctgccttcctggtttccttgactttggcctcctcctcttcctccttatcttctccaacctccttcctttatttgttccacagactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 14 - protein, 267 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLVRPPPSQVHSHCRPMPVQRTQCHIREDP

SEQ ID NO: 15 - DNA, 2427 bp, Homo sapiens
  CDS (219)..(494)
aaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctggggtgccaccaaattctgggcttggaacagcttcagctcccacccgctcctccacacacagacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctggtgaggcctcccccttcccaagtccattcccactgtaggccgatgcctgtgcaaaggacgcagtgccatatcagagaggatccttgaagaggactcaccccaagcaagggaaaattgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctacccatgtggcccaaaaaaaaaaaaaaaaaaaaaaaaaaaaaaaaaaaaaaaaaaaaaaaaaaaaaaaaaaaaa

SEQ ID NO: 16 - protein, 91 aa, Homo sapiens
MPVPWFLLSLALGRSPVVLSLERLVGPQDATHCSPGLSCRLWGATKFWAWNSFSSHPLLHTQTVTYSACLGTSCLLRAPCWRLRTCRQSWC

SEQ ID NO: 17 - DNA, 21 bp, Artificial
  siRNA for IL-17R
cagcggtctggttatcgtcta

SEQ ID NO: 18 - DNA, 21 bp, Artificial
  siRNA for IL-17R
cggcacctacgtagtctgcta

SEQ ID NO: 19 - DNA, 21 bp, Artificial
  siRNA for IL-17R
caggaaggtctggatcatcta

SEQ ID NO: 20 - DNA, 21 bp, Artificial
  siRNA for IL-17R
caggtttgagtttctgtccaa

SEQ ID NO: 21 - DNA, 21 bp, Artificial
  siRNA for IL-17RC
accgcagatcattaccttgaa

SEQ ID NO: 22 - DNA, 21 bp, Artificial
  siRNA for IL-17RC
caggtacgagaaggaactcaa

SEQ ID NO: 23 - DNA, 21 bp, Artificial
  siRNA for IL-17RC
cgggacttaaataaaggcaga

SEQ ID NO: 24 - DNA, 21 bp, Artificial
  siRNA for IL-17RC
ccgcgcggctctgctcctcta

SEQ ID NO: 25 - protein, 21 aa, Apis mellifera
MKFLVNVALVFMVVYISYIYA

SEQ ID NO: 26 - DNA, 2380 bp, Homo sapiens
  CDS (233)..(2350)
acactggccaaacaaaaacgaaagcactccgtgctggaagtaggaggagagtcaggactcccaggacagagagtgcacaaactacccagcacagccccctccgccccctctggaggctgaagagggattccagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgactcaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaacgtcactgtggacaaggttctcgagttcccattgctgaaaggccaccctaacctctgtgttcaggtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggactcccgcgccgggacgcggggtgggaccaggggcgggacctggggcgggggacgggacttaaataaaggcagacgctgtttttctaaaaaaa

SEQ ID NO: 27 - protein, 705 aa, Homo sapiens
MPVPWFLLSLALGRSPVVLSLERLVGPQDATHCSPGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 28 - DNA, 3373 bp, Homo sapiens
  CDS (78)..(2678)
gcggaaaagaaagcctcagaacgttcgctcgctgcgtccccagccggggccgagccctccgcgacgccacccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgtggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccactgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccctgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagccttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacattaaacgaacaggatgggccgggcacggtggctcacgcctgtaatcccagcacattgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaaaaaaaaaaa

SEQ ID NO: 29 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPVADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVHCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 30 - protein, 7 aa, Artificial
  Fusion protein linker
GSGSGSG

SEQ ID NO: 31 - DNA, 699 bp, Homo sapiens
  CDS (1)..(699)
gagcccaaatcttgtgacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 32 - protein, 232 aa, Homo sapiens
EPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 8 aa, Artificial
  Fusion protein linker
AGSGSGSG

SEQ ID NO: 34 - DNA, 477 bp, Mus musculus
  CDS (1)..(477)
atgagtccagggagagcttcatctgtgtctctgatgctgttgctgctgctgagcctggcggctacagtgaaggcagcagcgatcatccctcaaagctcagcgtgtccaaacactgaggccaaggacttcctccagaatgtgaaggtcaacctcaaagtctttaactcccttggcgcaaaagtgagctccagaaggccctcagactacctcaaccgttccacgtcaccctggactctccaccgcaatgaagaccctgatagatatccctctgtgatctgggaagctcagtgccgccaccagcgctgtgtcaatgcggagggaaagctggaccaccacatgaattctgttctcatccagcaagagatcctggtcctgaagagggagcctgagagctgccccttcactttcagggtcgagaagatgctggtgggtgtgggctgcacctgcgtggcctcgattgtccgccaggcagcctaa

SEQ ID NO: 35 - protein, 158 aa, Mus musculus
MSPGRASSVSLMLLLLLSLAATVKAAAIIPQSSACPNTEAKDFLQNVKVNLKVFNSLGAKVSSRRPSDYLNRSTSPWTLHRNEDPDRYPSVIWEAQCRHQRCVNAEGKLDHHMNSVLIQQEILVLKREPESCPFTFRVEKMLVGVGCTCVASIVRQAA

SEQ ID NO: 36 - DNA, 486 bp, Mus musculus
  CDS (1)..(486)
atgaagtgcacccgtgaaacagccatggtcaagtctttgctactgttgatgttgggacttgccattctgagggaggtagcagctcggaagaaccccaaagcaggggttcctgccttgcagaaggctgggaactgtcctcccctggaggataacactgtgagagttgacattcgaatcttcaaccaaaaccagggcatttctgtcccacgtgaattccagaaccgctccagttccccatgggattacaacatcactcgagacccccaccggttcccctcagagatcgctgaggcccagtgcagacactcaggctgcatcaatgcccagggtcaggaagacagcaccatgaactccgtcgccattcagcaagaaatcctggtccttcggagggagccccagggctgttctaattccttcaggttggagaagatgctcctaaaagttggctgcacctgtgtcaagcccattgtccaccaagcggcctaa

SEQ ID NO: 37 - protein, 161 aa, Mus musculus
MKCTRETAMVKSLLLLMLGLAILREVAARKNPKAGVPALQKAGNCPPLEDNTVRVDIRIFNQNQGISVPREFQNRSSSPWDYNITRDPHRFPSEIAEAQCRHSGCINAQGQEDSTMNSVAIQQEILVLRREPQGCSNSFRLEKMLLKVGCTCVKPIVHQAA